(19) United States
(12) Patent Application Publication
(10) Pub. No.: US 2015/0183834 A1
(43) Pub. Date: Jul. 2, 2015

(54) Filoviral Immunosuppressive Peptides

(71) Applicant: The Trustees of Columbia University in the City of New York, New York, NY (US)

(72) Inventors: W. Ian LIPKIN, New York, NY (US); Kavitha YADDANAPUDI, Union City, NJ (US); Gustavo PALACIOS, New York, NY (US)

(73) Assignee: The Trustees of Columbia University in the City of New York, New York, NY (US)

(21) Appl. No.: 14/266,355

(22) Filed: Apr. 30, 2014

Related U.S. Application Data
(63) Continuation of application No. 11/518,641, filed on Sep. 11, 2006, now abandoned.
(60) Provisional application No. 60/716,361, filed on Sep. 11, 2005.

(51) Int. Cl. C07K 14/005; G01N 33/50; C12N 7/00

(52) U.S. Cl. CPC C07K 14/005; C12N 7/00; G01N 33/5047; C12N 2760/14222; C12N 2760/14233; C12N 2760/14122; C12N 2760/14133; A61K 39/00

(57) ABSTRACT

The invention provides a region of strong secondary structure conservation between the C-terminus domain of the envelope glycoprotein of filoviruses and an immunosuppressive domain found in retroviral envelope glycoproteins. The invention provides filoviral peptides and modified derivatives thereof with strong immunosuppressive bioactivity. The invention further provides methods for treatment of autoimmune disorders by administering the immunosuppressive peptide. The invention also provides methods for the identification of therapeutic agents that modulate the immunosuppressive activity of the peptides. Antibodies against the inventive peptides and the modified derivatives thereof are also provided. Furthermore, the invention provides methods for treatment of filoviral infection by administering compositions comprising the antibodies and/or the therapeutic agents that modulate the immunosuppressive activity of the inventive peptides.

This application is a continuation of U.S. Ser. No. 11/518,641, filed Sep. 11, 2006, which claims the benefit of U.S. Provisional Ser. No. 60/716,361 filed on Sep. 11, 2005, the contents of which are hereby incorporated by reference.

The invention disclosed herein was made with U.S. Government support under NIH Grant Nos. AI 51292, A1056118, AI55466 and U54-AI057158. Accordingly, the U.S. Government has certain rights in this invention.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

### BACKGROUND OF THE INVENTION

Filoviruses are enveloped, non-segmented viruses with a negative-sense, single-stranded RNA genome of approximately 19 kb. Filoviral infections continue to present an unresolved obstacle in the epidemiology of infectious agents. Moreover, their acuteness is associated with consequent economic and social disruption, severely impacting the areas where the outbreak was epidemic. Ebola viruses (EBOY) cause hemorrhagic fever with mortality rates up to 88%. Since the initial outbreak in Zaire (now the Republic of Congo) in 1976, there have been more than 1,500 cases of human infection, with the most recent outbreaks occurring in Gabon and the Republic of the Congo in 2003. Together with Marburg virus (MARY), the four species of EBOV (Zaire, Sudan, Reston, Ivory Coast) comprise the family Filoviridae. Great apes are particularly susceptible to filovirus infection and EBOY and MARY have been implicated in the deaths of tens of thousands of chimpanzees and gorillas in central and western equatorial Africa. The natural reservoir of EBOY is unknown; preliminary data suggest that bats may be a reservoir for MARY. There is no established therapy for either EBOY or MARY. Ebola and Marburg viruses can cause hemorrhagic fever (HF) outbreaks with high mortality in primates. Whereas Marburg (MARV), Ebola Zaire (ZEBOV) and Ebola Sudan (SEBOV) viruses are pathogenic in humans, apes, and monkeys, Ebola Reston (REBOV) is pathogenic only in monkeys. Early immunosuppression may contribute to pathogenesis by facilitating viral replication. Lymphocyte depletion, intravascular apoptosis and cytokine dysregulation are prominent in fatal cases.

There are little experimental data on MARV pathogenesis; however, clinical reports indicate that it is likely to be similar to EBOV. Infection with EBOV results in hypotension, coagulopathy, and hemorrhage, culminating in fulminant shock. Primary target cells for infection include mononuclear phagocytic cells, in which the virus lytically replicates. Vascular instability is likely caused by virus-induced activation of mononuclear phagocytic cells and the subsequent production of active mediator molecules, such as proinflammatory cytokines and chemokines. Recent data indicate that these target cells are activated early upon infection and that activation is independent of virus replication. Although all viral components may contribute to disease the filoviral glycoproteins are thought to be major pathogenic determinants. There is evidence that the filovirus glycoproteins play an important role in cell tropism, the spread of infection and pathogenicity. Biosynthesis of the transmembrane glycoprotein involves a series of co- and post-translational events, including proteolytic cleavage by a host cell protease.

Furthermore, a marked depression in immunity appears to be an important factor in the pathology of the filovirus haemorrhagic fever. Immunosuppression is observed in EBOV infected cynomolgous macaques that is not directly associated with virus production. Dendritic cells in lymphoid tissues are identified as early and sustained targets of infection; bystander lymphocyte apoptosis occurs in intravascular and extravascular locations (Geisbert et al., 2003). Apoptosis and loss of NK cells are prominent findings, suggesting the importance of innate immunity in determining the fate of the host. CD4+ and CD8+ lymphocyte counts decrease 60-70% during the first 4 days after infection. Among CD8+ lymphocytes, this decline is more pronounced among the CD8^{lo} population, which is composed mostly of CD3− CD16+ NK cells. In contrast, the number of CD20+ B lymphocytes in the blood does not change significantly. Analysis of peripheral blood mononuclear cell gene expression indicates temporal increases in tumor necrosis factor-related apoptosis-inducing ligand and Fas transcripts, revealing a possible mechanism for the observed bystander apoptosis. Neither mice nor guinea pigs exhibit the hemorrhagic manifestations that characterize EBOV infections of primates. Furthermore, lymphocyte apoptosis, is not observed in mice or guinea pigs (Bray et al., 1998; Connolly et al., 1999(.

Studies from the early 1990s have reported a sequence similarity between the C-terminal domain of the filovirus glycoprotein and the immunosuppressive domain of the envelope protein from retroviruses (Volchkov et al., 1992; Bukreyev et al., 1993). Retroviral infections often cause severe immunosuppression in many species, and accumulating evidence supports the view that retroviral protein components may play an important role in this immune dysfunction. In vitro investigations have shown that inactivated retroviruses or transmembrane envelope protein p15E as well as a synthetic 17-amino acid peptide (CKS-17) are highly immunosuppressive (Good et al., 1991; Haraguchi et al., 1992(a); Haraguchi et al., 1995 (a); Haraguchi et al., 1995(b); Haraguchi et al., 1993; Haraguchi et al., 1992(b); Ogasawara et al., 1990; Ogasawara et al., 1988; Ogasawara et al., 1991).

However, there are no experimental data related to filoviruses and mechanisms of immunosuppression. Furthermore, there remains an urgent need for useful vaccines and treatments of filoviral infection.

### SUMMARY OF THE INVENTION

The invention provides, an isolated peptide comprising the consecutive amino acid sequence of any one of SEQ ID NOS: 1-84, 108-376, wherein the total length of the peptide is less than about 66, 64, 62, 60, 58, 56, 54, 52, 50, 48, 46, 44, 42, 40, 38, 36, 34, 32, 30, 28, 26, 24, 22, 20, 18 amino acids and wherein the peptide has immunosuppressive activity.

The invention provides, an isolated therapeutic peptide comprising: NRXX(X1)DXL(X2)X(R)XXXXC sequence motif, wherein X is any amino acid, (X1) is leucine, or isoleucine, (X2) is leucine, isoleucine, or phenylalanine, (R) is arginine or lysine, wherein the peptide length is from about 16 amino acids to about 66, 64, 62, 60, 58, 56, 54, 52, 50, 48, 46, 44, 42, 40, 38, 36, 34, 32, 30, 28, 26, 24, 22, 20, 18 amino acids.

The invention provides an isolated therapeutic peptide, the peptide comprising: NRXX(X1)DXL(X2)X(R)WGGTC sequence motif, wherein X is any amino acid, (X1) is leucine, or isoleucine, (X2) is leucine, isoleucine, or phenylalanine, (R) is arginine or lysine, wherein the peptide length is from about 16 amino acids to about 66, 64, 62, 60, 58, 56, 54, 52, 50, 48, 46, 44, 42, 40, 38, 36, 34, 32, 30, 28, 26, 24, 22, 20, 18 amino acids.

The invention provides an isolated therapeutic peptide, the peptide comprising: 1/LL/INRXX(X1)DXL(X2)X(R)WGGTC sequence motif, wherein I/L and L/I indicates that the position can have either amino acid, X is any amino acid, (X1) is leucine, or isoleucine, (X2) is leucine, isoleucine, or phenylalanine, (R) is arginine or lysine, wherein the peptide length is from about 18 amino acids to about 66, 64, 62, 60, 58, 56, 54, 52, 50, 48, 46, 44, 42, 40, 38, 36, 34, 32, 30, 28, 26, 24, 22, 20, 18 amino acids.

The invention provides an isolated peptide, wherein the peptide dimerizes with another peptide selected from the group of peptides of SEQ ID NOS:1-84, 108-376, wherein the total length of each peptide of the dimer is less than 26 amino acids and wherein the dimer has immunosuppressive activity. In certain aspects, the dimer comprises two identical peptides. In certain aspects, the dimer comprises two different peptides. In other aspects, the peptide is attached to a detectable marker, a carrier molecule, or is conjugated at a free amine group with a polyalkylene glycol, such as polyethylene glycol.

The invention provides a method for modulating or suppressing an immune response of a subject, the method comprising administering to the subject any one of the inventive peptides in an effective amount so as to suppress the immune response in the subject. In certain aspects, the subject suffers from an autoimmune disease.

In certain aspects, the present invention provides isolated immunosuppressive peptides from filoviruses. In other aspects, the invention provides isolated therapeutic, including, immunosuppressive peptides from filoviruses. In other aspects, the invention provides methods for identifying agents that can be useful for treating filoviral infections. In other aspects, the invention also provides methods for modulating immune response in a subject, methods for suppressing immune response in a subject, and/or induction of immunosuppression in a subject suffering from autoimmune diseases or inflammatory disorders.

The invention provides an isolated peptide having amino acid sequence of SEQ ID NO:1 or having an amino acid sequence which is at least about 70%, 72%, 74%, 76%, 78%, 80%, 82%, 84%, 86%, 88%, 90%, 91%, 92%, 93%, 94%, 95%, 96%, 97%, 98%, 99%, 99.5% identical to the amino acid sequence set forth in SEQ ID NO:1. The invention also provides an isolated peptide having amino acid sequence of SEQ ID NO:2 or having an amino acid sequence which is at least about 70%, 72%, 74%, 76%, 78%, 80%, 82%, 84%, 86%, 88%, 90%, 91%, 92%, 93%, 94%, 95%, 96%, 97%, 98%, 99%, 99.5% identical to the amino acid sequence set forth in SEQ ID NO:2. The invention also provides an isolated peptide having amino acid sequence of SEQ ID NO:3 or having an amino acid sequence which is at least about 70%, 72%, 74%, 76%, 78%, 80%, 82%, 84%, 86%, 88%, 90%, 91%, 92%, 93%, 94%, 95%, 96%, 97%, 98%, 99%, 99.5% identical to the amino acid sequence set forth in SEQ ID NO:3. The invention also provides an isolated peptide having amino acid sequence of SEQ ID NO:4 or having an amino acid sequence which is at least about 70%, 72%, 74%, 76%, 78%, 80%, 82%, 84%, 86%, 88%, 90%, 91%, 92%, 93%, 94%, 95%, 96%, 97%, 98%, 99%, 99.5% identical to the amino acid sequence set forth in SEQ ID NO:4. The invention also provides an isolated peptide having amino acid sequence of SEQ ID NO:5 or having an amino acid sequence which is at least about 70%, 72%, 74%, 76%, 78%, 80%, 82%, 84%, 86%, 88%, 90%, 91%, 92%, 93%, 94%, 95%, 96%, 97%, 98%, 99%, 99.5% identical to the amino acid sequence set forth in SEQ ID NO:5. The invention provides an isolated peptide having amino acid sequence of SEQ ID NO:108 or having an amino acid sequence which is at least about 70%, 72%, 74%, 76%, 78%, 80%, 82%, 84%, 86%, 88%, 90%, 91%, 92%, 93%, 94%, 95%, 96%, 97%, 98%, 99%, 99.5% identical to the amino acid sequence set forth in SEQ ID NO:113. The invention provides an isolated peptide having amino acid sequence of SEQ ID NO:108 or having an amino acid sequence which is at least about 70%, 72%, 74%, 76%, 78%, 80%, 82%, 84%, 86%, 88%, 90%, 91%, 92%, 93%, 94%, 95%, 96%, 97%, 98%, 99%, 99.5% identical to the amino acid sequence set forth in SEQ ID NO:113. The invention provides an isolated peptide having amino acid sequence of SEQ ID NO:115 or having an amino acid sequence which is at least about 70%, 72%, 74%, 76%, 78%, 80%, 82%, 84%, 86%, 88%, 90%, 91%, 92%, 93%, 94%, 95%, 96%, 97%, 98%, 99%, 99.5% identical to the amino acid sequence set forth in SEQ ID NO:115. The invention provides an isolated peptide having amino acid sequence of SEQ ID NO:117 or having an amino acid sequence which is at least about 70%, 72%, 74%, 76%, 78%, 80%, 82%, 84%, 86%, 88%, 90%, 91%, 92%, 93%, 94%, 95%, 96%, 97%, 98%, 99%, 99.5% identical to the amino acid sequence set forth in SEQ ID NO:117. The invention provides an isolated peptide having amino acid sequence of SEQ ID NO:121 or having an amino acid sequence which is at least about 70%, 72%, 74%, 76%, 78%, 80%, 82%, 84%, 86%, 88%, 90%, 91%, 92%, 93%, 94%, 95%, 96%, 97%, 98%, 99%, 99.5% identical to the amino acid sequence set forth in SEQ ID NO:121.

The invention further provides an isolated peptide having an amino acid sequence selected from the group of sequences with SEQ ID NOS: 6-84, 108-376. In certain aspects, the invention provides an isolated therapeutic peptides having amino acid sequence which is at least about 70%, 72%, 74%, 76%, 78%, 80%, 82%, 84%, 86%, 88%, 90%, 91%, 92%, 93%, 94%, 95%, 96%, 97%, 98%, 99%, 99.5% identical to the amino acid sequence set forth in SEQ ID NO: 6-84, 108-376. In other aspects, the invention also provides an isolated peptide comprising the consecutive amino acid sequence of any one of SEQ ID NOS:1-84, 108-376 wherein the total length of the peptide is less than 26, 27, 28, 29, 30, 31, 32, 33, 34, 25, 36, 37, 38, 39, 40, 41, 42, 43, 44, 45 amino acids. In other aspects, the invention also provides an isolated peptide having the consecutive amino acid sequence of any one of SEQ ID NOS:1-84, 108-376 wherein the total length of the peptide is less than 26, 25, 24, 23, 22, 21, 20, 19, 18 amino acids.

The invention provides an isolated peptide selected from the group consisting of sequences with SEQ ID NOS:1 to 84, or any other peptide of the invention. The invention also provides an isolated peptide comprising from about 9, 10, 11, 12, 13, 14, 15 to about 11, 12, 13, 14, 15, 17, 19, 21, 23, 25, 27, 29, 31, 33, 35, 37, 39, 41, 43, 45, 50, 55, 60, 65, 70, 75 consecutive amino acids from a polypeptide, for example but not limited to a filoviral glycoprotein polypeptide, wherein at least a portion of the amino acid sequence can form a coiled-coil secondary structure. In certain aspects, the consecutive amino acids comprise an amino acid sequence motif RXXXD wherein X can be any amino acid; an amino acid sequence motif comprising two arginines separated from each other by at least eight amino acids, such as RXXXDXXXXD, wherein the RXXXD motif is between the two arginines. In other aspects, the consecutive amino acids can form a secondary structure similar or identical to the secondary structure of the carboxy terminus domain of the retroviral env protein. In other aspects, the isolated peptide specifically binds to a T cell receptor, wherein the peptide is not the CKS17 peptide (SEQ ID NO: 86) or the P15E peptide (SEQ ID NO: 87). The invention provides an isolated peptide comprising at least 9, 10, 11, 12, 13, 14, 15, 16, 17 consecutive amino acid residues of any one of SEQ ID NOS:1-85, wherein the peptide has an immunosuppressive bioactivity, wherein the length of the peptide is from about 9, 10, 11, 12, 13, 14, 15, 16, 17, 18, 19, 20, 21, 22, 23, 24, 25, 26 amino acids to about 10, 11, 12, 13, 14, 15, 16, 17, 18, 19, 20, 21, 22, 23, 24, 25, 26, 27, 28, 29, 30, 31, 33, 35, 37, 39, 41, 43, 45 amino acids, and wherein the peptide has therapeutic, including immunosuppressive bioactivity. The invention provides an isolated peptide comprising 15, 16, 17, 18 consecutive amino acid residues of any one of SEQ ID NOS:108-376, wherein the peptide has an immunosuppressive bioactivity, wherein the length of the peptide is from about 15, 16, 17, 18, 19, 20, 21, 22, 23, 24, 25, 26 amino acids to about 18, 19, 20, 21, 22, 23, 24, 25, 26, 27, 28, 29, 30, 31, 33, 35, 37, 39, 41, 43, 45 amino acids, and wherein the peptide has therapeutic, including immunosuppressive, bioactivity.

The invention provides an isolated therapeutic peptide comprising: NRXX(X1)DXL(X2)X(R)XXXXC sequence motif, wherein X is any amino acid, (X1) is leucine, or isoleucine, (X2) is leucine, isoleucine, or phenylalanine, (R) is arginine or lysine, wherein the peptide length is from about 16 amino acids to about 26 amino acids.

The invention provides an isolated therapeutic peptide comprising: NRXX(X1)DXL(X2)X(R)WGGTC sequence motif, wherein X is any amino acid, (X1) is leucine, or isoleucine, (X2) is leucine, isoleucine, or phenylalanine, (R) is arginine or lysine, wherein the peptide length is from about 16 amino acids to about 26 amino acids.

The invention provides an isolated therapeutic peptide from filovirus, wherein the peptide is capable of binding to a CD4+ and/or a CD8+ T-cells. The invention provides a monomer of any one of the peptides of the invention, including but not limited to SEQ ID NOS:1-84, 108-376 or any other peptide of the invention. The invention provides a dimer comprising one of any one of the peptides of the invention, including but not limited to SEQ ID NOS: 1-84, 108-376. In one aspect, the dimer comprises a disulfide bond. In another aspect, the dimer comprises two different monomers selected from the group of peptides of SEQ ID NOS: 1-84, 108-376. In another aspect, the dimer comprises two identical monomers selected from the group of peptides of SEQ ID NOS: 1-84, 108-376. In another aspect, a peptide of the invention has a sequence that is from about 90% to about 100% identical to an amino acid sequence of a Marburg virus, a Reston Ebola virus, a Zaire Ebola virus, a Sudan Ebola virus, an Ivory Coast Ebola virus or any combination thereof.

The present invention provides an immunosuppressive peptide with amino acid sequence of any one of SEQ ID NOS:1 to 84, or any other peptide of the invention, wherein the immunosuppressive peptide is not CKS17 or P15E. In one embodiment, the peptide is derived from the glycoprotein polypeptide sequence of Ebola Zaire. In another embodiment, the peptide is derived from the glycoprotein polypeptide sequence of Ebola Reston. In another embodiment, the peptide is derived from the glycoprotein polypeptide sequence of Ebola Ivory Coast. In yet another embodiment, the peptide is derived from the glycoprotein polypeptide sequence of Ebola Sudan. In another embodiment, the peptide is derived from the glycoprotein polypeptide sequence of the Marburg filovirus.

The invention also contemplates a peptide which comprises from about at least 9, 11, 13, 15, 16, 17, 18 to about 16, 17, 18, 19 consecutive amino acids from any one of the amino acid sequences listed in SEQ ID NOS:1-84, 108-376 or any other peptide of the invention, wherein the peptide is less than 65, 60, 55, 50, 45, 35, 30, 26, 25, 24, 23, 22, 21, 20, 19 amino acids long.

In one aspect the invention provides a peptide, which may be at least 75% identical to a peptide of any one of SEQ ID NOS:1-84, 108-376, or any other peptide of the invention. In one embodiment the homology can be between 75% and 79.99%. In another embodiment the homology can be between 80% and 84.99%. In another embodiment the homology can be between 85% and 89.99%. In another embodiment the homology can be between 90% and 94.99%. In another embodiment the homology can be between 95% and 99.99%.

The invention also provides an isolated peptide which have amino acid sequence comprising from about 9 to about 17, 19, 21, 23, 25, 27, 29, 31, 33, 35, 40, 45, 50, 55, 60 consecutive amino acids that are 65% to 69.5%. 70% to 74.5%, 75% to 79.5%, 80% to 84.5%, 85% to 89.5%, 90% to 94.5%, 95% to 99.99% identical to a polypeptide sequence from the glycoprotein of Ebola Zaire, Ebola Reston, Ebola Sudan, Ebola Ivory Coast, or Marburg virus. The invention also provides peptides which have amino acid sequence that is 90%, 91%, 92%, 93%, 94%, 95%, 96%, 97%, 98%, 99%, 99.99% identical to a polypeptide sequence of the glycoprotein from Ebola Zaire, Ebola Reston, Ebola Sudan, Ebola Ivory Coast, or Marburg virus. In another embodiment, the invention provides isolated, therapeutic, immunosuppressive peptides that have the immunosuppressive amino acid sequence from glycoprotein polypeptide variants of Ebola Zaire, Ebola Reston, Ebola Sudan, Ebola Ivory Coast, or Marburg virus.

In another aspect, the invention provides an immunosuppressive peptide of any one of SEQ ID NOS:1-84, 108-376, or any other peptide of the invention, wherein the peptide binds to a CD4+ T-cell, and/or CD8+ T cell, and/or to a CD8^{lo} cell.

In another aspect, the invention provides an isolated peptide with immunosuppressive bioactivity, wherein the peptide is modified. Modifications contemplated by the invention preserve the immunosuppressive bioactivity of the peptide. In one embodiment, the modification can be a polyalkylene glycol conjugated to the peptide. In another embodiment, the polyalkylene glycol is polyethylene glycol. In one embodiment, the polyethylene glycol molecule can be straight. In another embodiment, the polyethylene glycol molecule can be branched. Polyethylene glycol molecules of various molecular weight are contemplated by the invention.

In another aspect, the peptide of the invention can be linked to a detectable marker which can be a chemical label such as, but not limited to, radioactive isotopes, fluorescent groups, chemiluminescent label, colorimetric label, an enzymatic marker, and affinity moieties such as biotin that facilitate detection of the labeled peptide. In another embodiment, the peptide can be dye-labeled with fluoresceins or rhodamine conjugates. Other modifications can include incorporation of rare amino acids, dextra (D)-amino acids, glycosylation sites, cytosine for specific disulfide bridge formation.

In one aspect, the isolated peptide is a monomer. In another aspect, the invention provides a peptide, which is a dimer. The dimer can be composed of monomers that are identical or different. The dimer can be composed of peptides, which are modified, by any of the possible modifications described herein. In another aspect, the dimer comprises a disulphide bond.

In one aspect, the invention provides an isolated peptide, wherein the peptide is linked to a carrier molecule. In another aspect, the invention provides an isolated peptide, wherein the peptide is conjugated at a free amine group with a polyalkylene glycol, including but not limited to polyethylene glycol. In another aspect, the invention provides an isolated peptide, which is comprised in a composition comprising a pharmaceutically acceptable carrier.

In one aspect, the invention provides an isolated nucleic acid encoding isolated therapeutic peptide of any on of SEQ ID NOS: 1-84, 108-376. Provided is an isolated nucleic acid having nucleic acid sequence selected from the group of sequences with SEQ ID NOS: 88-107. In another aspect, the invention provides an expression vector comprising a nucleic acid encoding a peptide of the invention. Provided are also, compositions comprising the expression vectors.

In another aspect, the invention provides an isolated nucleic acids encoding any one of the peptides of SEQ ID NOS:1-84, 108-376, or any other peptide of the invention. For example, the invention provides nucleic acids in SEQ ID NOS: 88-107. It is understood that due to the degeneracy of the genetic code, several nucleic acids can encode the same amino acid. The invention further provides an expression vector comprising a nucleic acid encoding any one of the peptides of SEQ ID NOS:1-84, 108-376, or any other peptide of the invention. In one embodiment, the expression vector can be useful for recombinant expression of the inventive peptide. In another embodiment, the expression vector can be useful as a delivery vehicle for peptide expression in a subject undergoing treatment with the immunosuppressive peptide.

In another aspect, the invention provides an antibody which binds specifically to any one of the peptides of SEQ ID NOS:1-84, 108-376, or any other peptide of the invention. In one aspect, the antibody is at least bivalent, i.e., the antibody has two binding sites that have the same specificity. In another embodiment, the antibody can comprise an antibody subsequence or fragment. The antibody fragment can comprise for example a (Fab′)2 molecule. In one embodiment, the antibody can be monoclonal. In another embodiment, the antibody can be polyclonal. In one embodiment, the antibody can be humanized. In another embodiment, the antibody can be fully-human. The invention further provides a method for the treatment of filoviral infection in a subject, the method comprising administering to the subject an antibody which binds specifically to the immunosuppressive peptide.

In another aspect, the invention provides a method for modulating, and/or suppressing an immune response in a subject, the method comprising administering an effective amount of the inventive peptide to the subject so as to suppress the immune response of the subject. The peptide of the invention can be administered in a composition that comprises the peptide. In another embodiment, the peptide can be administered in a composition that comprises an expression vector that comprises a nucleic acid, which encodes a peptide of the invention. A variety of routes of administration of the peptide are contemplated by the invention, and these routes include but are not limited to parenteral, oral, intratracheal, sublingual, pulmonary, topical, rectal, nasal, buccal, sublingual, vaginal, or via an implanted reservoir.

In another aspect, the invention provides a method for treating an autoimmune disease in a subject, the method comprising administering to a subject an effective amount of any one of the inventive peptides, including but not limited to peptides of SEQ ID NO: 1-84, 108-376, so as to suppress subject's immune response, wherein the autoimmune disease is one or more of diabetes mellitus, rheumatoid arthritis, multiple sclerosis, systemic lupus erythematosis, myasthenia gravis, scleroderma, inflammatory bowel disease, Crohn's disease, ulcerative colitis, Hashimoto's thyroiditis, Graves' disease, Sjogren's syndrome, polyendocrine failure, vitiligo, peripheral neuropathy, rejection of transplantation, graft-versus-host disease, autoimmune polyglandular syndrome type I, acute glomerulonephritis, Addison's disease, adult-onset idiopathic hypoparathyroidism (AOIH), alopecia totalis, amyotrophic lateral sclerosis, ankylosing spondylitis, autoimmune aplastic anemia, autoimmune hemolytic anemia, Behcet's disease, Celiac disease, chronic active hepatitis, CREST syndrome, dermatomyositis, dilated cardiomyopathy, eosinophilia-myalgia syndrome, epidermolisis bullosa acquisita (EBA), giant cell arteritis, Goodpasture's syndrome, Guillain-Barre syndrome, hemochromatosis, Henoch-Schonlein purpura, idiopathic IgA nephropathy, juvenile rheumatoid arthritis, Lambert-Eaton syndrome, linear IgA dermatosis, myocarditis, narcolepsy, necrotizing vasculitis, neonatal lupus syndrome (NLE), nephrotic syndrome, pemphigoid, pemphigus, polymyositis, primary sclerosing cholangitis, psoriasis, rapidly-progressive glomerulonephritis (RPGN), Reiter's syndrome, stiff-man syndrome, thyroiditis. In certain aspects the subject is a human, a primate, a mouse, a rat, a fish, a dog, a pig, and the like.

In another aspect, the invention provides a method for identifying an agent that modulates the immunosuppressive bioactivity of any one of the peptides of SEQ ID NOS:1-84, 108-376, or any other peptide of the invention. In certain aspects, the method for identifying an agent that modulates an immunosuppressive bioactivity of any one of the peptides of SEQ ID NOS:1-84, 108-376, can comprise: (a) contacting a cell, which can be stimulated by mitogens, and which is exposed to any one of the immunosuppressive peptides of the invention with an agent, (b) determining whether the cell exhibits an inhibited or an increased immune response, as measured by any of the assays provided herein or any other suitable assays known in the art, wherein exhibition of increased immune response is indicative of an agent that modulates the immunosuppressive effect of the peptide. In certain aspects, the contacting step can be performed when a cell is treated with any one of the immunosuppressive peptides of the invention with an agent. Treatment of a cell with a polypeptide of the invention can be done before the agent is added. Treatment of a cell with a polypeptide of the invention can be done after the agent is added. Treatment of a cell with a polypeptide of the invention can be done concomitantly with the addition of the agent. In certain aspects, the method can use any suitable cell, wherein the cell is a CD4+ cell, CD8+ cell, or a cell in a population of cells as comprised in PBMCs, or a mixture thereof. In certain aspects, the determining step comprises comparing cell proliferation or levels of cytokines produced by the cell in the presence of the agent with the levels determined in the absence of the agent.

In one embodiment, an agent that modulates the immunosuppressive bioactivity can bind directly to the immunosuppressive peptide, for example but not limited to an antibody that binds to the peptide, wherein the biding of the agent to the peptide can modulate the immunosuppressive bioactivity. In another embodiment, an agent may modulate the immunosuppressive bioactivity without biding to the inventive peptide, for example by binding a cellular receptor to which the peptide would typically bind. In one aspect, agents that inhibit the immunosuppressive bioactivity of the inventive peptides can be useful for the treatment of filoviral infections. Any suitable cell, which can be used to determine immunosuppressive activity of a peptide can be used in the present method. In certain embodiments, the cell can be a CD4+, CD8+, a cell comprised in the population of PBMCs, or any combination thereof. In certain embodiments, the cell can be from an isolated, and/or clonal cell line.

In certain embodiments, a determining step of the methods for identifying an agent that modulates the immunosuppressive activity of a peptide, can be any suitable assay or method, including but not limited to the methods described herein, to determine immunosuppressive activity of a peptide. The determining step can include a comparison between the effect produced by an immunosuppressive peptide in the presence and/or absence of a candidate agent. In certain embodiments, the method for identifying an agent that modulates the immunosuppressive bioactivity can be automated and/or high throughput method. In certain aspects, combinatorial libraries of small molecule compounds can be screened in the methods to identify agents, which modulate the immunosuppressive bioactivity of a peptide. In other aspects, various biological agents can be screened to identify an agent that modulated the immunosuppressive bioactivity.

In another aspect, the invention provides a method for the treatment of disorders associated with hyperproliferation of lymphocytes, the method comprising administering to a subject an effective amount of any one of the immunosuppressive peptides of SEQ ID NOS:1-87, 108-376, or any other peptide of the invention. In another aspect, the invention provides a method for treating a subject suffering from a lympho-proliferative disorder or disease, including but not limited to T-cell lymphomas, the method comprising administering to the subject an effective amount of any one of the therapeutic peptides of the invention, for example but not limited to any one of the peptides of SEQ ID NO: 1-84, 108-376. In other aspect, the invention provides method treating a subject suffering from a disorder or disease, characterized by wanted proliferation of cells of bone marrow lineage, the method comprising administering to the subject an effective amount of any one of the therapeutic peptides of the invention, for example but not limited to any one of the peptides of SEQ ID NO: 1-84, 108-376.

### BRIEF DESCRIPTION OF THE FIGURES

FIG. 1 shows a comparison of the amino acid sequence of a mouse retroviral glycoprotein (from Moloney-Murine Leukemia Virus) with the amino acid sequence of Ebola virus glycoprotein. This figure illustrates the secondary structure conservation as indicated by the distribution of helical (H) and coil (C) motifs between the C-terminus domain of the glycoprotein of Ebola virus and the immunosuppressive domain found in Moloney-Murine Leukemia Virus.

FIG. 2 shows sequence alignment of immunosuppressive peptides.

FIGS. 3A-B show a computer generated 3-dimensional image of the location of the immunosuppressive domain in the Ebola virus glycoprotein. FIG. 3A represents a dimerized form of the Ebola virus glycoprotein and FIG. 3B shows a close-up view of the immunosuppressive domain.

FIG. 4 shows the results of an in vitro assay which demonstrates the immunosuppressive characteristic of the Ebola virus glycoprotein peptide and the CKS-17 peptide from the Moloney-Murine Leukemia Virus. Briefly, PBMCs were treated in vitro with the mitogen PHA and either no peptide (control), or 30 μM of each of the reverse sequence CKS-17 peptide, CKS-17 peptide, or the Ebola virus glycoprotein peptide of SEQ ID NO: 1. Following a 12 hour incubation period at 37 degrees celcius, cytokine production by the PBMC was determined and the results are displayed on the histograms. “Control” indicates that no peptide was added to the culture, LIP8975 is a peptide that has an amino acid sequence which is the reverse of the amino acid sequence of CKS17, LIP8974 is a peptide that has amino acid sequence identical to CKS-17, and LIP8972 is a peptide that has amino acid sequence identical to SEQ ID NO:1. The graphs show protein concentration of IL-2, IL-12p40 and IL-10 in supernatants of PBMC cells treated with the corresponding peptides.

FIG. 5A shows an electron micrograph of Marburg virus (left) and organization of filoviral proteins in the viral ribonucleoprotein complex (RNP). The RNP consists of the non-segmented negative stranded RNA genome and four of the structural proteins, nucleoprotein (NP), virion structural protein (VP) 30, VP35, L (large or polymerase) protein. VP24 and VP40 are membrane associated proteins, and the spikes are formed by the glycoprotein (GP). The Ebola and Marburg structural proteins show different electrophoretic mobility which is schematically illustrated.

FIG. 5B shows the genomic organization of the Ebola RNA genome.

FIGS. 6A-D show depletion and inactivation of human T lymphocytes following exposure to inactivated filovirus or filoviral peptides. PBMC were exposed to filoviral peptides, inactZEBOV, or neither peptide nor virus for 48 hours in the presence of anti-CD3/CD28. After staining with antibodies to CD4 and CD8, cells were analyzed by flow cytometry. (FIG. 6A) Dot plots of CD4+ versus CD8+ lymphocytes following activation with anti-CD3/CD28 alone (control), or anti-CD3/CD28 and ZEBOV peptide. Experiments were performed with PBMC from Five different donors; data from one representative donor are shown. Numbers in quadrants represent the percentages of each subpopulation. (FIG. 6B) Percentages of CD4+ and CD8+ lymphocyte subsets in PBMC following activation with anti-CD3/CD28 antibodies alone or anti-CD3/CD28 and either filoviral peptides or inactZEBOV. Results are expressed as percentage of total PBMC. Values represent mean±SD calculated from five different donors in each sample group. (FIG. 6C) Representative histogram showing cell surface expression of CD4 and CD8 markers on PBMC activated with anti-CD3/CD28 alone, or anti-CD3/CD28 and ZEBOV peptide. Numbers indicate the mean fluorescence intensity of CD4 or CD8 expression+standard deviation (SD). Values were derived from five donors in each sample group. (FIG. 6D) Absolute numbers of CD4+ and CD8+ T cells following activation with anti-CD3/CD28 alone or anti-CD3/CD28 and filoviral peptide. Data represent mean±SD calculated from five different donors in each sample group. Double asterisk indicates p<0.01 (relative to control samples; ANOVA and Dunnett's test for multiple comparisons).

FIGS. 7A-C show inactivation of human T lymphocytes following exposure to inactivated filovirus or filoviral peptides. PBMC were exposed to filoviral peptides, inactZEBOV, or neither peptide nor virus for 12 or 48 hours in the presence of anti-CD3/CD28. After staining with antibodies to CD4, CD8, CD25 and CD69, cells were analyzed by flow cytometry. (FIG. 7A) Histograms represent expression of activation markers in PBMC activated with anti-CD3/CD28 alone or anti-CD3/CD28 and ZEBOV peptide. Data were obtained from five different donors; data from one representative donor are shown. Numbers in gates represent the percentages of CD25 or CD69 positive cells in the CD4+ T or CD8+ T cell subpopulations, respectively. (FIG. 7B) Percentages of CD25 or CD69 positive cells in CD4+ and CD8+ lymphocyte subsets following activation with anti-CD3/CD28 alone or anti-CD3/CD28 in the presence of inactZEBOV or filoviral peptide. Results are expressed as percentages of CD4+ or CD8+ T cells. Values represent mean±SD calculated from five different donors in each sample group. Data for CD69 expression were obtained 12 and 48 hours after activation. Asterisk indicates p<0.05; double asterisk indicates p<0.01 (relative to control samples; ANOVA and Dunnett's test for multiple comparisons). (FIG. 7C) Representative histogram showing cell surface expression of CD25 and CD69 markers on PBMC activated with anti-CD3/CD28 alone, or anti-CD3/CD28 and ZEBOV peptide. The mean fluorescence intensity is represented as a percentage of the maximum expression. Data were obtained from five different donors; data from one representative donor are shown.

FIGS. 8A-C show defective proliferation and cell cycle progression in human T lymphocytes following exposure to filoviral peptides. PBMC were exposed to ZEBOV peptide, REBOV peptide, or no peptide for 48 hours in the presence of anti-CD3/CD28. After staining with antibodies to CD4, CD8 and BrdU, cells were analyzed by flow cytometry. (FIG. 8A) Dot plots of PBMC activated with anti-CD3/CD28 alone or anti-CD3/CD28 and ZEBOV peptide. Data were obtained from five different donors; data from one representative donor are shown. Numbers in quadrants represent the percentages of each subpopulation. (FIG. 8B) Percentage of BrdU+ cells in CD4+ and CD8+ lymphocyte subsets following activation with anti-CD3/CD28 alone or antiCD3/CD28 and either ZEBOV peptide or REBOV peptide. Results are expressed as percentages of total PBMC. Values represent mean±SD for five different donors in each sample group. (FIG. 8C) Cell cycle analysis of PBMC activated with anti-CD3/CD28 or anti-CD3/CD28 and either ZEBOV peptide or REBOV peptide. Cells were stained with 7-AAD and analyzed by flow cytometry. The percentages of cells in G1, S, G2 and hypodiploid phases of the cell cycle are represented as a table. Values indicate mean±SD calculated from five different donors in each sample group. Asterisk indicates p<0.01 (relative to control samples; ANOVA and Dunnett's test for multiple comparisons).

FIGS. 9A-E show that filoviral peptide exposure induces human T cell apoptosis. PBMC were exposed to filoviral peptides, inactZEBOV, or neither peptide nor virus in the presence of anti-CD3/CD28. After staining with antibodies to CD4, CD8, AnnexinV-FITC (marker for apoptosis) and PI (marker for apoptosis or necrosis), cells were analyzed by flow cytometry. (FIG. 9A) Percentages of AnnexinV-FITC+ PI− cells in gated CD4+ or CD8+ cells following treatment with anti-CD3/CD28 antibodies alone or anti-CD3/CD28 and inactZEBOV for 48 hours. Values represent mean±SD calculated from five different donors in each sample group. (FIG. 9B) Strategy for gating PBMC. Viable PBMC were gated according to forward scatter (FSC) and side scatter (SSC) profile (R1 gate). (FIG. 8C) Live (R1) cells were further gated according to CD4 expression and FSC (R5). Dot plots of AnnexinV-FITC and PI fluorescence on gated CD4+ cells. (FIG. 8D) Live (R1) cells were further gated according to CD8 expression and FSC (R5). Dot plots of AnnexinVFTTC and PI fluorescence on gated CD8+ cells. For both (FIG. 9C) and (FIG. 9D), numbers in quadrants represent the percentages of each subpopulation. Experiments were performed with PBMC from five different donors; results obtained from one donor are shown. (FIG. 9E) Percentages of AnnexinV-FITC+ PI− cells in gated CD4+ or CD8+ cells following treatment with anti-CD3/CD28 antibodies alone or anti-CD3/CD28 and filoviral peptide for 12 hours. Values represent mean±SD calculated from five different donors in each sample group. Double asterisk indicates p<0.01 (relative to control samples; ANOVA and Dunnett's test for multiple comparisons).

FIGS. 10A-C show that exposure of human PBMC to ZEBOV peptide results in decreased release of IFN-γ, IL-2, IL-12p40, TNF-α, IL-1β and MCP-1, and increased release of IL-10. PBMC were exposed to 1, 20 or 401M of ZEBOV peptide, REBOV peptide or no peptide for 48 hours in the presence of anti-CD3/CD28. Cytokines were assayed in cell supernatant using Luminex technology. Results indicate mean concentration (pg/ml)±SD. Values were obtained from five different donors in each sample group. Asterisk indicates p<0.05; double asterisk indicates p<0.01 (relative to control samples; Kruskal-Wallis non-parametric ANOVA with Dunn's multiple comparison test).

FIGS. 11A-E show that exposure of rhesus macaque PBMC to REBOV peptide results in depletion and inactivation of CD4+ and CD8+ T cells. Rhesus PBMC were exposed to ZEBOV peptide, REBOV peptide or no peptide for 48 hours in the presence of anti-human CD3ε. (FIG. 11A) Dot plots of CD4 versus CDS lymphocytes in activated PBMC exposed to ZEBOV peptide or REBOV peptide. Experiments were performed with PBMC from five different donors; results obtained from one donor are shown. Numbers in quadrants represent the percentages of each subpopulation. (FIG. 11B) Absolute numbers of CD4+ and CD8+ T cells following activation of PBMC with anti-CD3e alone or anti-CD3e and either ZEBOV peptide or REBOV peptide. Data represent mean±SD calculated from five macaques in each sample group. (FIG. 11C) Percentages of CD4+ and CD8+ lymphocyte subsets in PBMC following activation with anti-CD3ε antibody alone or anti-CD3ε and either ZEBOV peptide or REBOV peptide. Results are expressed as percentages of total PBMC. Values represent mean±SD calculated from five macaques in each sample group. (FIG. 11D) Percentages of CD69+CD4+ and CD69+CD8+ subsets in PBMC following activation with anti-CD3ε or anti-CD3ε and either ZEBOV peptide or REBOV peptide. Results are expressed as percentages of CD4+ or CD8+ T cells. Values represent mean±SD calculated from five macaques in each sample group. Asterisk indicates p<0.05; double asterisk indicates p<0.0 (relative to control samples; ANOVA and Dunnett's test for multiple comparisons). (FIG. 11E) Representative histogram showing cell surface expression of CD69 markers on rhesus PBMC activated with anti-CD3ε, or anti-CD3ε and either ZEBOV peptide or REBOV peptide. The mean fluorescence intensity is represented as a percentage of the maximum expression. Data were obtained from five different donors; data from one representative donor are shown.

FIGS. 12A-B show that exposure of Rhesus macaque PBMC to REBOV peptide results in apoptosis of CD4+ and CD8+ T cells and in decreased release of IFN-γ and IL-2. (FIG. 12A) PBMC were exposed to filoviral peptide, inactZEBOV, or neither peptide nor virus in the presence of anti-CD3e. After staining with antibodies to CD4, CD8, AnnexinV-FITC (marker for apoptosis) and P1 (marker for apoptosis or necrosis) cells were analyzed by flow cytometry. Percentages of AnnexinV-FITC+ PI− cells in gated CD4+ or CD8+ T cells following treatment with anti-CD3e alone or anti-CD3ε and inactZEBOV, ZEBOV peptide or REBOV peptide. Values represent mean±SD calculated from five macaques in each sample group. Asterisk indicates p<0.01 relative to control samples (ANOVA and Dunnett's test for multiple comparisons). (FIG. 12B) PBMC were exposed for 48 hours to 1, 20 or 401M of ZEBOV peptide, REBOV peptide or no peptide in the presence of anti-CD3ε. Cytokines in cell supernatants were assayed by flow cytometry (Luminex). Results indicate mean concentration (pg/ml)±SD. Asterisk indicates p<0.05; double asterisk indicates p<0.01 (relative to control samples; Kruskal-Wallis non-parametric ANOVA with Dunn's multiple comparison test).

### DETAILED DESCRIPTION OF THE INVENTION

The term “therapeutically effective amount” used interchangeably with the term “effective amount” as used herein means that amount of a compound, material, such as the peptides of the present invention, or composition comprising a compound of the present invention which is effective for producing some desired therapeutic effect by modulating, immune response in at least a sub-population of cells in a subject, and thereby modulating immune response of subject at a reasonable benefit/risk ratio applicable to any medical treatment.

The terms “treatment” or “treat” as used herein include treating, preventing, ameliorating, and/or decreasing the severity of the symptoms of a disease or disorder, or improving prognosis for recovery.

Filoviruses cause hemorrhagic fevers with high levels of fatality. They are classified in two genera within the family Filoviridae: Ebola virus (EBOV) and Marburg virus (MARV). Four species of Ebola virus are currently recognized: Zaire, Sudan, Reston and Ivory Coast. Ebola virus species Zaire (ZEBOV) and Sudan (SEBOV) as well as Marburg (MARV), are highly pathogenic in human and nonhuman primates, with case fatality levels of up to 90%. Ebola virus species Reston (REBOV) is pathogenic in monkeys but does not cause disease in humans or great apes. Fatal outcome in filoviral infection is associated with an early reduction in the number of circulating T cells, failure to develop specific humoral immunity, and the release of pro-inflammatory cytokines. The membrane anchored filoviral glycoprotein (GP) is present on the surface of virions and infected cells; GP mediates receptor binding and fusion. Filoviral GPs are considered to be major viral pathogenic determinants and contribute to both immunosuppression and vascular dysregulation (Yang et al, 2000; Volchkov et al., 2001; Feldman et al., 2001).

The transmembrane glycoproteins of many animal and human retroviruses share structural features including a conserved region that has strong immunosuppressive properties (Denner et al., 1994; Haraguchi et al., 1995). CKS17, a synthetic peptide corresponding to this domain in oncogenic retroviruses, has been used to dissect the pathophysiology of immunosuppression (Cinaciolo et al., 1985; Haraguchi et al, 1995(a). CKS17 causes an imbalance of human type-1 and type-2 cytokine production, suppresses cell-mediated immunity (Haraguchi et al, 1995(b), and blocks the activity of protein kinase C, a cellular messenger involved in T cell activation (Gottlieb et al., 1990; Kadota et al., 1991). During the course of establishing a microbial sequence database to support development of tools for surveillance and differential diagnosis of infectious diseases, a region of strong secondary structure conservation between the C-terminal domain of the envelope glycoprotein of filoviruses and CKS17 was discovered. An alignment of the filoviral glycoprotein and retroviral immunosuppressive domains illustrated primary sequence similarity between a wide range of retroviruses and filoviruses. Importantly, three cysteine residues implicated in disulfide bonding were also conserved, reinforcing similarities at the level of secondary structure. Provided herein is functional analysis of the putative immunosuppressive domain in various species of EBOV and MARV and demonstration that the immunosuppressive effect of different species of GP peptides is consistent with pathogenicity observed in different animal hosts.

Filoviruses cause hemorrhagic fever with very high mortality rates. Although other viral components may contribute to disease the filoviral glycoproteins are thought to be the major pathogenic determinants. A marked depression of immunity is observed in Ebola virus infected cynomolgus macaques, which is not directly associated with virus production. The invention provides a region of strong secondary structure conservation between the C-terminus domain of the envelope glycoprotein of filoviruses and an immunosuppressive domain found in retroviral envelope glycoproteins. In certain aspects, the primary amino acid sequences of the filoviral peptides of the invention differ from the primary amino acid sequence of CKS17 of Moloney murine leukemia virus, or pE15. The invention provides filoviral peptides and modified derivatives thereof with strong immunosuppressive bioactivity. The invention further provides methods for treatment of autoimmune disorders by administering the immunosuppressive peptide. The invention also provides methods for the identification of therapeutic agents that modulate the immunosuppressive activity of the peptides. Antibodies against the inventive peptides and the modified derivatives thereof are also provided. Furthermore, the invention provides methods for treatment of filoviral infection by administering compositions comprising the antibodies and/or the therapeutic agents that modulate the immunosuppressive activity of the inventive peptides.

In certain aspects, the invention provides a 17 amino acid domain in filoviral glycoproteins that resembles an immunosuppressive motif in retroviral envelope proteins. In other aspects, the invention provides methods to functionally characterize a peptide comprising such amino acid domain. In certain embodiments, activated human or rhesus peripheral blood mononuclear cells (PBMC) were exposed to inactivated ZEBOV (also referred to inactivZEBOV), or a panel of 17mer peptides representing all sequenced strains of filoviruses, and then analyzed for CD4+ and CD8+ T cell activation, and/or apoptosis, and/or cytokine expression. In certain aspects, the invention provides that exposure of human and rhesus PBMC to ZEBOV, SEBOV or MARV peptides or inactivated ZEBOV resulted in decreased expression of activation markers on CD4 and CD8 cells; CD4 and CD8 cell apoptosis as early as 12 hours post exposure; inhibition of CD4 and CD8 cell cycle progression; decreased IL-2, IFN-γ, and IL12-p40 expression; and increased IL-10 expression. Only rhesus T cells were sensitive to REBOV peptides. These findings are consistent with the observation that REBOV is not pathogenic in humans, and have implications for understanding the pathogenesis of filoviral HF.

Ebola and Marburg viruses can cause hemorrhagic fever (HP) outbreaks with high mortality in primates. Whereas MARV, ZEBOV and SEBOV are pathogenic in humans, apes, and monkeys, REBOV is pathogenic only in monkeys. Early immunosuppression may contribute to pathogenesis by facilitating viral replication. The filoviral proteins VP35 and VP24 have immunomodulatory effects. VP35 inhibits induction of IFN α and β by blocking phosphorylation and nuclear translocation of interferon regulatory factor-3 (Basler et al., 2000, Basler et al., 2002). ZEBOV VP24 interacts with karyopherin al, the nuclear localization signal receptor for PY-STAT1 (Reid et al., 2006). Active virus replication is prerequisite for the immunosuppressive effects of VP35 and VP24. In certain aspects, the invention provides that the immunosuppressive effects for filoviral GP sequences as observed and described herein are independent of viral replication.

In certain aspects, the invention provides 17mer filoviral peptides from ZEBOV, SEBOV or MARV. These peptides had a strong immunosuppressive influence on anti-CD3/CD28 activated human PBMC. Furthermore, activated CD4+ and CD8+ T cells failed to upregulate activation markers on their surface and exhibited reduced cell-cycle progression. CD4+ and CD8+ T cell dysfunction may stem from immune inactivation following direct contact with the peptide. Alternatively, the effect may be the indirect result of inadequate stimulation by the antigen presenting cells. In vitro studies of ZEBOV have revealed suppression of immune responses within infected monocyte/macrophages and endothelial cells (Gupta et al., 2001, Harcourt et al., 1998). Dendritic cells infected with ZEBOV are functionally impaired and only poorly stimulate T cells (Mahanty et al., 2003, Bosio et al., 2003). IFN α/β production has been shown to influence dendritic cell functions. VP35 protein of ZEBOV suppresses the induction of IFN α/β and may indirectly contribute to inhibition of dendritic cell functions (Basler at al., 2003).

T cells do not support filoviral replication (Basler et al, 2004). The observation that inactZEBOV can induce T cell apoptosis in PBMC cultures is consistent with earlier studies indicating that virus replication is not a prerequisite for T cell apoptosis (Geisbert et al., 2000, Hensley et al., 2002). Potential mechanisms for T cell apoptosis in PBMC cultures treated with filoviral peptides of the invention include direct interaction of peptides with the cell surface or indirect effects mediated by soluble factors released from monocytes exposed to these peptides. Studies of purified human CD4+ and CD8+ T cells indicate that ZEBOV peptide alone is sufficient to induce activation and cell death in either population. It is conceivable that both direct and indirect mechanisms may be implicated in T cell apoptosis.

The influence of ZEBOV peptide on Th1 and Th2-related cytokine production was examined by stimulated PBMCs using Luminex technology. Whereas T helper type 1 cells predominantly produce IFN-γ, T helper type 2 cells secrete IL-4, IL-5 and IL-10. IL-12, a cytokine produced by monocytes/macrophages, enhances cell-mediated immunity (D'Andrea et al., 1992, Wolf et al., 1991). IL-10 is mainly produced by monocytes/macrophages and T cells; it inhibits activation of T-helper lymphocytes either directly (41) or by suppressing activation of antigen presenting cells (Ding et al., 1993). High plasma levels of IL-10 are reported in Filovirus-infected patients with fatal outcome (Villinger et al., 1999).

In certain aspects, the invention provides that the 17mer ZEBOV peptide suppresses expression of the type 1 cytokines IL-12 and IFN-γ, while enhancing expression of the type 2 cytokine IL-10. Enhanced expression of IL-10 and reduced expression of IL-12 likely imbalances Th1- and Th2-related cytokine production and suppress cell-mediated immunity. Haraguchi et al. (1995) have demonstrated that CKS-17, a retroviral peptide, acts directly on monocytes/macrophages and differentially modulates the production of IL-10 and IL-12. Furthermore, a neutralizing anti-human IL-10 monoclonal antibody blocks the peptide-mediated inhibition of IFN-γ, supporting the hypothesis that inhibition of IFN-γ production may be secondary to increase in IL-10 and depression in IL-12 levels produced by the retroviral peptide. Similar cytokine-mediated cross-regulation may be implicated in filoviral immunosuppression.

Proinflammatory cytokines and chemokines play a vital role in one of the earliest phases of the host resistance to viral and microbial infections by participating in various cellular and inflammatory processes. In certain aspects, the invention provides that 17mer filoviral peptides decreased secretion in PBMC cultures of proinflammatory cytokines TNFα and IL-1β and chemokine MCP-1. These defective inflammatory responses may be associated with impaired T-cell activation observed in peptide treated lymphocytes. Non-fatal ZEBOV infection is associated with early inflammatory responses (Baize et al., 2002). The observed peptide mediated cytokine inhibition as described herein, suggests that filoviral transmembrane glycoprotein and peptides thereof as provided by the invention may be involved in suppressing the onset of early inflammatory responses that are crucial for controlling viral spread in filoviral infections.

All African EBOV subtypes (ZEBOV, SEBOV and Ivory Coast) cause a severe hemorrhagic disease in humans and nonhuman primates with extraordinarily high fatality rates. The fourth subtype, REBOV, which was initially isolated from cynomolgus monkeys, is non-pathogenic in humans and appears to be a lethal pathogen only for nonhuman primates (Jahlring et al., 1996). In certain aspects, the invention provides that exposure of human PBMC to REBOV peptides had no effect on markers of CD4+ or CD8+ activation, viability, or cytokine levels in cell supernatants. Whereas human PBMC were sensitive to ZEBOV but not REBOV, monkey PBMC were sensitive to both ZEBOV and REBOV. These findings demonstrate that strain specific differences in peptide sequence determine immunological effects on PBMC in vitro, and correlate with the pathogenic potential of ZEBOV, SEBOV and MARV viruses versus REBOV virus in human and non-human primates.

The rapidly progressing high fatality HF associated with EBOV and MARV infections is accompanied by profound immunosuppression and vascular dysfunction. Several factors likely contribute to the severity of disease. These viruses quickly replicate and cause cytotoxicity in a wide range of cells and tissues within the body, and the viral glycoprotein (particularly the mucin-like domain) has been implicated in this cytotoxicity (Yang et al., 2000). Recent studies have also demonstrated an immunosuppressive effect of the viral VP35 protein in inhibiting interferon regulatory factor (IRF)-3 activation and induction of IFN a and 13 as well as other antiviral responses (Basler et al., 2003, Hartman et al., 2004). Data described herein show that in addition to contributing to HF pathogenicity through cytotoxicity, filoviral glycoproteins also have a potent immunosuppressive effect. The 17 amino acid motif described herein dysregulates Th 1 and Th2 responses and depletes CD4 and CD8 T-cells through apoptosis. Investigation of interactions between filoviral glycoproteins and the host immune system may allow development of specific strategies to reduce the extreme morbidity and mortality associated with HF due to EBOV and MARV infections.

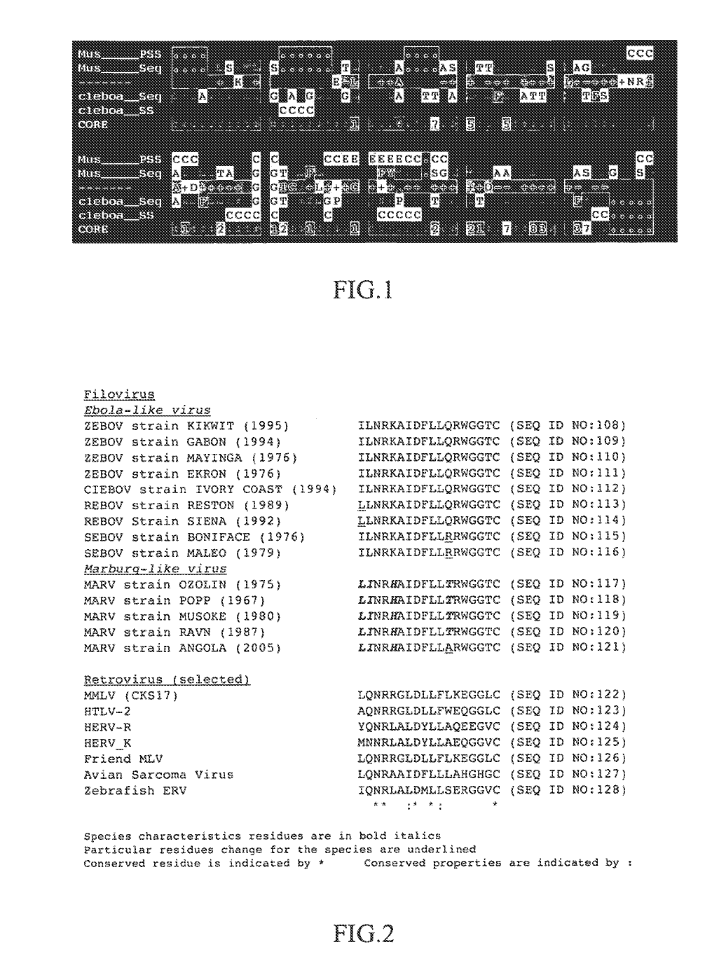

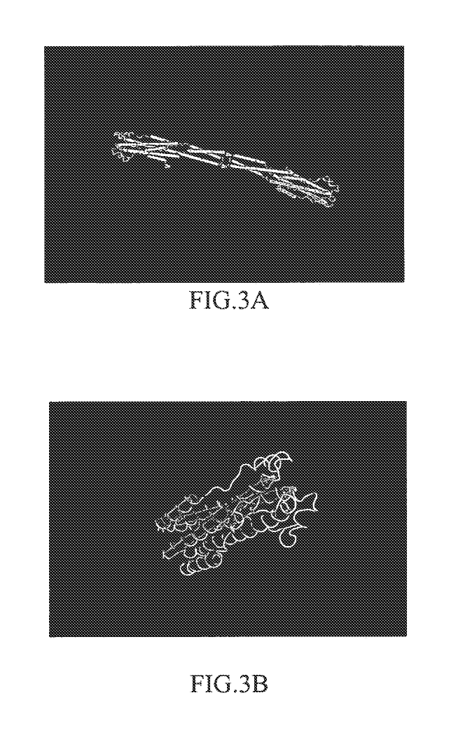

In certain aspects, the invention provides isolated, therapeutic, including immunosuppressive, peptides comprising consecutive amino sequences derived from glycoprotein polypeptide from filoviruses. In one aspect, the invention provides a peptide with the amino acid sequence of ILNRKAIDFLLQRWGGT (SEQ ID NO:1). In one embodiment the peptide of SEQ ID NO:1 is located within the envelope of filoviruses that resembles in secondary structure an immunosuppressive domain found in some retroviruses (FIG. 1). FIG. 2 is an alignment of filovirus and retroviral immunosuppressive peptides that illustrates similarity between a range of HERVs and filoviruses. Additional amino acids, including downstream cysteine residues implicated in disulfide bonding are also conserved, suggesting similarities at the level of secondary structure (Benit et al., 2001). FIG. 3 displays a model of the trimeric structure of the filovirus envelope wherein the region proposed to mediate immunosuppression occupies a prominent and exposed position on the virion surface.

In one aspect, the peptide of the invention may be derived from consecutive amino acids at positions 584 to 600 at the C-terminal end in the envelope glycoprotein of Ebola Zaire (Accession No: P87671). In another aspect, the peptide may have an amino acid sequence of at least 74.99% identity to the amino acid sequence of SEQ ID NO:1. In another embodiment, the peptide can have between 75% and 79.99% identity to the amino acid sequence of SEQ ID NO:1. In another embodiment, the peptide can have between 80% and 84.99% identity to the amino acid sequence of SEQ ID NO:1. In another embodiment, the peptide can have between 85% and 89.99% identity to the amino acid sequence of SEQ ID NO:1. In yet another embodiment, the peptide can have between 90% and 94.99% identity to the amino acid sequence of SEQ ID NO:1. In still another embodiment, the peptide can have between 95% and 99% identity to the amino acid sequence of SEQ ID NO:1.

The peptide may have an amino acid sequence motif similar to the RXXXD sequence motif, found in TGF-β, wherein X is any amino acid. In one embodiment the RXXXD motif is in the N-terminal half of the peptide.

The peptide of the invention may also result in a coiled-coil secondary structure which is conserved with the secondary structure of the immunosuppressive domain found in retroviral envelopes (e.g. the secondary structure of CKS-17 from Moloney-Murine Leukemia virus). The coiled-coil secondary structure of the peptide of the invention is similar to the secondary structure of the CKS-17 peptide, despite the low sequence similarity, ˜30%, between the primary amino acid sequence of the inventive peptide compared to CKS17. The peptide of the invention may also specifically bind to a T cell receptor.

The peptide of the invention may also contain two arginines a sequence motif RXXXDXXXXR, wherein the arginines are separated from each other by eight amino acids in the primary amino acid sequence of the peptide. In one embodiment the first or N-terminal arginine of the two arginines is part of the RXXXD sequence motif found in TGF-β. In other embodiments the second arginine can be a lysine.

In another aspect, the invention provides a therapeutic peptide from about 15, 16, 18, 20, 22, 24, 26 amino acids to about 15, 16, 18, 20, 22, 24, 26, 28, 30, 32, 34, 36, 38, 40, 45, 50, 55, 60, 65, 70 amino acids, which peptide comprises a NRXX(X1)DXL(X2)X(R)XXXX sequence motif, wherein X is any amino acid; (X1) indicates any hydrophobic amino acid, for example but not limited to leucine, or isoleucine; (X2) indicates any hydrophobic amino acid, for example but not limited to leucine, or isoleucine, or an aromatic amino acid such as phenylalanine; (R) indicates any positively charged amino acid, included but not limited to arginine, or lysine; (R) can also be alanine, glutamine or glutamic acid. In another aspect, the invention provides a therapeutic peptide from about 16, 18, 20, 22, 24, 26 amino acids to about 16, 18, 20, 22, 24, 26, 28, 30, 32, 34, 36, 38, 40, 45, 50, 55, 60, 65, 70 amino acids, which peptide comprises a NRXX(XI)DXL(X2)X(R)XXXXC sequence motif, wherein (X1), (X2) and (R) are described herein. In another aspect, the invention provides a therapeutic peptide from about 18, 20, 22, 24, 26 amino acids to about 18, 20, 22, 24, 26, 28, 30, 32, 34, 36, 38, 40, 45, 50, 55, 60, 65, 70 amino acids, which peptide comprises a I/LL/INRXX(X1)DXL(X2)X(R)WGGTC sequence motif, wherein UL and L/I indicates that the position can have either amino acid, (X1), (X2) and (R) are described herein. Exemplary peptides which comprise NRXX(X1)DXL(X2)X(R)XXXX are illustrated by SEQ ID NOS: 1-87. Exemplary peptides which comprise sequence NRXX(X1)DXL(X2)X(R)XXXXC motif are illustrated by SEQ ID NOS: 108-128, and SEQ ID NOS: 129-376.

In one aspect, the invention provides a peptide with the amino acid sequence LINRHAIDFLLTRWGGT (SEQ ID NO:2) which is derived from the sequence of Marburg strain of filovirus.

In one aspect, the invention provides a peptide with the amino acid sequence ILNRKAIDFLLQRWGGT (SEQ ID NO:3) which is derived from Ebola Sudan virus.

In one aspect, the invention provides a peptide with the amino acid sequence LLNRKAIDFLLQRWGGT (SEQ ID NO:4) which is derived from Ebola Reston virus.

In one aspect, the invention provides a peptide with an amino acid sequence ILNRKAIDFLLQRWGGT (e.g., SEQ ID NO:5) which is derived from Ebola Ivory coast virus.

In certain aspects of the invention, the isolated therapeutic peptide of the invention comprises from about 9, 10, 11, 12, 13, 14, 15, 16, 17, 18, 19, 20 to about 10, 11, 12, 13, 14, 15, 16, 17, 19, 21, 23, 25, 27, 29, 31, 33, 35, 37, 41, 43, 45, 50, 55, 60 amino acid residues and includes a sequence motif RXXXDXXXXR. In certain aspects of the invention, the isolated therapeutic peptide of the invention comprises from about 15, 16, 17, 18, 19, 20 to about 15, 16, 17, 19, 21, 23, 25, 27, 29, 31, 33, 35, 37, 41, 43, 45, 50, 55, 60 amino acid residues and includes a sequence motif NRXX(X1)DXL(X2)X(R)XXXX. In certain aspects of the invention, the isolated therapeutic peptide of the invention comprises from about 16, 17, 18, 19, 20 to about 16, 17, 19, 21, 23, 25, 27, 29, 31, 33, 35, 37, 41, 43, 45, 50, 55, 60 amino acid residues and includes a sequence motif NRXX(X1)DXL(X2)X(R)XXXXC. In certain aspects of the invention, the peptide is about 9 consecutive amino acid residues. In certain aspects of the invention, the peptide is about 11 consecutive amino acid residues. In certain aspects of the invention, the peptide is about 13 consecutive amino acid residues. In certain aspects of the invention, the peptide is about 15 consecutive amino acid residues. In certain aspects of the invention, the peptide is about 16 consecutive amino acid residues. In certain aspects of the invention, the peptide is about 17 consecutive amino acid residues. In certain aspects of the invention, the peptide is about 18 consecutive amino acid residues. In certain aspects of the invention, the peptide is about 19 consecutive amino acid residues. In certain aspects of the invention, the peptide is about 20 consecutive amino acid residues. In certain aspects of the invention, the peptide is about 21 consecutive amino acid residues. In certain aspects of the invention, the peptide is about 22 consecutive amino acid residues. In certain aspects of the invention, the peptide is about 23 consecutive amino acid residues. In certain aspects of the invention, the peptide is about 24 consecutive amino acid residues. In certain aspects of the invention, the peptide is about 25 consecutive amino acid residues. In certain aspects of the invention, the peptide is about 26 consecutive amino acid residues. In certain aspects of the invention, the peptide is about 28 consecutive amino acid residues. In certain aspects of the invention, the peptide is about 30 consecutive amino acid residues. In certain aspects of the invention, the peptide is about 32 consecutive amino acid residues. In certain aspects of the invention, the peptide is about 34 consecutive amino acid residues. In certain aspects of the invention, the peptide is about 36 consecutive amino acid residues. In certain aspects of the invention, the peptide is about 38 consecutive amino acid residues. In certain aspects of the invention, the peptide is about 40 consecutive amino acid residues. In certain aspects of the invention, the peptide is about 42 consecutive amino acid residues. In certain aspects of the invention, the peptide is about 44 consecutive amino acid residues. In certain aspects of the invention, the peptide is about 46 consecutive amino acid residues.

(SEQ ID NO: 6)
LQNRRGLDLLFLKEGGL
(SEQ ID NO: 7)
LQNRRGLDLLFLKEGGL
(SEQ ID NO: 8)
LQNRRGDLLFLKEGGL
(SEQ ID NO: 9)
LQNRRGLDLLFLREGGL
(SEQ ID NO: 10)
LQNRRGLDLLFLKEGGL
(SEQ ID NO: 11)
LQNRXGLDLLFLSQGEL
(SEQ ID NO: 12)
LQNCRGLDLLFLSQGGL
(SEQ ID NO: 13)
LQNRRGLDLLFLSQGGL
(SEQ ID NO: 14)
AQNRRGLDLLF[X]EQGGL
(SEQ ID NO: 15)
LQNRRGLDLLTAEQGGI
(SEQ ID NO: 16)
LQNRRGLDLLTAEQGGX
(SEQ ID NO: 17)
LQNRRGLDMLTAAQGGI
(SEQ ID NO: 18)
LQNYQELDELTAAQRET
(SEQ ID NO: 19)
LQNCQGLDMLMAAQGGI
(SEQ ID NO: 20)
LQNRXGLDLLTAEKGGL
(SEQ ID NO: 21)
LQNCRGLDLLTAEKGGP
(SEQ ID NO: 22)
LQNRRGLDLLTAEKGGL
(SEQ ID NO: 23)
LQNHRGLNLLTAEKGRL
(SEQ ID NO: 24)
LQNRRGLNMLTAEKRGL
(SEQ ID NO: 25)
LQNRKGLDLLTAEKGSL
(SEQ ID NO: 26)
LQNRKGLNLLTAEKGGL
(SEQ ID NO: 27)
LQNRRGP[X]LLTAEKGGL
(SEQ ID NO: 28)
FQNCRGLDLLTAEKGGL
(SEQ ID NO: 29)
LQNCXGLDLLTVEEGGF
(SEQ ID NO: 30)
LQNRALDLLIAKRGGT
(SEQ ID NO: 31)
LQNRRALDLLTAKRGGT
(SEQ ID NO: 32)
LQNRRALDLLTAERGGT
(SEQ ID NO: 33)
LQNQRALNLLTAEQGGT
(SEQ ID NO: 34)
LQNRRALDLLTAEQGGT
(SEQ ID NO: 35)
LQNQRALDLLAAEKGSP
(SEQ ID NO: 36)
AQNRRALDLLTAEKGGT
(SEQ ID NO: 37)
AQNRQALDLLMAEKGRT
(SEQ ID NO: 38)
LNNRLAVDYLLAQVGEV
(SEQ ID NO: 39)
LNNRLALXXLLTEQSXA
(SEQ ID NO: 40)
LNNRLMLDCLLAVXGRI
(SEQ ID NO: 41)
LQNQLTXEVLPAEGGT
(SEQ ID NO: 42)
LQNQHALDVLTTKAGGT
(SEQ ID NO: 43)
AQNRQALDVITAEVGGT
(SEQ ID NO: 44)
AQNRQALDVLTTEVXGT
(SEQ ID NO: 45)
MQNRQALDILMAKVGGT
(SEQ ID NO: 46)
WENRLQLDIILAEKGVV
(SEQ ID NO: 47)
WENKIALNIILAVNGSV
(SEQ ID NO: 48)
XENRMAIGNILAEKGRV
(SEQ ID NO: 49)
WENRIALDMTLAKEGGV
(SEQ ID NO: 50)
WENKIALDMIPAKEGGD
(SEQ ID NO: 51)
LQNRMALDILTAAPGGT
(SEQ ID NO: 52)
LQNHMALDILTVAQGGT
(SEQ ID NO: 53)
LQNCMALDTLSAAQSET
(SEQ ID NO: 54)
LQNRMSLDIVTTAQGG
(SEQ ID NO: 55)
LQNWMALDIVTADQGGT
(SEQ ID NO: 56)
LQNQMALDILTAPQGGT
(SEQ ID NO: 57)
LQNCMALDIFMAAQEGT
(SEQ ID NO: 58)
LQNHMALDTLIAAQGGT
(SEQ ID NO: 59)
LYNHMALDILIAAQGGT
(SEQ ID NO: 60)
LXNRMALDILTAAQGGT
(SEQ ID NO: 61)
LQNRMALDILTAAEGGT
(SEQ ID NO: 62)
LQNQMALDMLTATQGGV
(SEQ ID NO: 63)
LQNHVAPDMLTAAQGGV
(SEQ ID NO: 64)
LQNQMALHILTAAQGRV
(SEQ ID NO: 65)
LQNRAAIDFLLLAHGHG
(SEQ ID NO: 66)
YQNRLPLDXLLAEESGV
(SEQ ID NO: 67)
YQNRLALDYLLAEEGGV
(SEQ ID NO: 68)
YQNRLALDYLLAQEEGV
(SEQ ID NO: 69)
YQNRLALDYLLAQEGGV
(SEQ ID NO: 70)
YQNRLGLDYLLAQEGGI
(SEQ ID NO: 71)
YXNRLALDYHLASEGRV
(SEQ ID NO: 72)
YQNRLALDYLLALEGGV
(SEQ ID NO: 73)
YQNRLALDYLLASEGGV
(SEQ ID NO: 74)
YQNRLALDYLLAAEGGV
(SEQ ID NO: 75)
YQNRLALNYLLAAEGG-
(SEQ ID NO: 76)
YQNRLALDYLIAAEGGV
(SEQ ID NO: 77)
LFNRHAIDFLLTRWGGT
(SEQ ID NO: 78)
LAVERYLKDQQLLGIWG
(SEQ ID NO: 79)
LELGQDVANLKTRNSTK
(SEQ ID NO: 80)
LWLGEQVXSLQLQRQLR
(SEQ ID NO: 81)
IXMEDRTINLKHQLEVQ
(SEQ ID NO: 82)
IWLGDRMMNLEHXMQLQ
(SEQ ID NO: 83)
IWMGDRLMSLEHRFQLQ
(SEQ ID NO: 84)
DLAEEQIGVLHQMAQLG

In the above-described SEQ ID NOS, X stands for any amino acid.

In one embodiment the invention provides peptide that is not the CKS17 peptide with amino acid sequence LQNRRGLDLLFLKEGGL (SEQ ID NO:86) or the P15E peptide with amino acid sequence (SEQ ID NO:87).

In another embodiment, the invention provides a nucleic acid sequence encoding any one of the peptides of the invention. Because of the degeneracy of the genetic code, several possible nucleic acid sequences may code for each peptide of the invention. For example, the invention may include nucleic acid sequence, wherein in the below described SEQ ID NOS, Y stands for any nucleotide:

(SEQ ID: 88)
AUU UUA AAU CGC AAA GCU AUU GAU UUU UUA UUA CAA
CGC UGG GGC GGC ACU UGA.

In yet another embodiment the invention provides a nucleic acid sequence having one or of the following sequences:

(SEQ ID: 89)
UUA CAA AAU CGC CGC GGC UUA GAU UUA UUA UUU UUA
AAA GAA GGC GGC UUA UGA
(SEQ ID: 90)
UUA CAA AAU CGC CGC GGC GAU UUA UUA UUU UUA AAA
GAA GGC GGC UUA UGA
(SEQ ID: 91)
UUA CAA AAU CGC CGC GGC UUA GAU UUA UUA UUU UUA
CGC GAA GGC GGC UUA UGA
(SEQ ID: 92)
UUA CAA AAU CGC CGC GGC UUA GAU UUA UUA UUU UUA
AAA GAA GGC GGC UUA UGA
(SEQ ID: 93)
UUA CAA AAU CGC [YYY] GGC UUA GAU UUA UUA UUU
UUA UCU CAA GGC GAA UUA UGA
(SEQ ID: 94)
UUA CAA AAU UGU CGC GGC UUA GAU UUA UUA UUU UUA
UCU CAA GGC GGC UUA UGA
(SEQ ID: 95)
UUA CAA AAU CGC CGC GGC UUA GAU UUA UUA UUU UUA
UCU CAA GGC GGC UUA UGA
(SEQ ID: 96)
UUA CAA AAU CGC CGC GGC UUA GAU UUA UUA ACU GCU
GAA CAA GGC GGC AUU UGA
(SEQ ID: 97)
UUA CAA AAU CGC CGC GGC UUA GAU AUG UUA ACU GCU
GCU CAA GGC GGC AUU UGA
(SEQ ID: 98)
UUA CAA AAU UAU CAA GAA UUA GAU GAA UUA ACU GCU
GCU CAA CGC GAA ACU UGA
(SEQ ID: 99)
UUA CAA AAU UGU CGC GGC UUA GAU UUA UUA ACU GCU
GAA AAA GGC GGC CCU UGA
(SEQ ID: 100)
UUA CAA AAU CGC CGC GGC UUA AAU AUG UUA ACU GCU
GAA AAA CGC GGC UUA UGA
(SEQ ID: 101)
UUU CAA AAU UGU CGC GGC UUA GAU UUA UUA ACU GCU
GAA AAA GGC GGC UUA UGA
(SEQ ID: 102)
UUA CAA AAU CGC CGC GCU UUA GAU UUA UUA GGC GCU
AAA CGC GGC GGC ACU UGA
(SEQ ID: 103)
UUA CAA AAU CAA CGC GCU UUA AAU UUA UUA CGU ACU
GCU GAA GGC GGC ACU UGA
(SEQ ID: 104)
UUA CAA AAU CAA CGC GCU UUA GAU UUA UUA GCU GCU
GAA AAA GGC UCU CCU UGA
(SEQ ID: 105)
GCU CAA AAU CGC CAA GCU UUA GAU UUA UUA AUG GCU
GAA AAA GGC CGC ACU UGA
(SEQ ID: 106)
UUA AAU AAU CGC UUA GCU GUU GAU UAU UUA UUA GCU
CAA GUU GGC GAA GUU UGA
(SEQ ID: 107)
UUA CAA AAU CAA CAU GCU UUA GAU GUU UUA ACU ACU
AAA GCU GGC GGC ACU UGA

The peptide can contain amino acids with charged side chains, such as acidic and basic amino acids. In addition, these peptides may contain one or more D-amino acid residues in place of one or more L-amino acid residues provided that the incorporation of the one or more D-amino acids does not abolish all or so much of the activity of the peptide that it cannot be used in the compositions and methods of the invention. Incorporating D-amino acids in place of L-amino acids is favorable as it may provide additional stability to a peptide.

Chemically synthesized peptides carry free termini thus being electrically charged. In one embodiment, the peptide of the invention is capped at the amino or carboxy terminus, or both termini. Modification of the N- and/or C-terminus can lead to increased stability, increased permeability in cells, and/or increased activity. Examples of amino terminal capping group include but are not limited to a lipoic acid moiety, which can be attached by an amide linkage to the amino terminus of a peptide. Another example of an amino terminal capping group useful in the peptides described herein is an acyl group, which can be attached in an amide linkage to the alpha-amino group of the amino terminal amino acid residue of a peptide.

In addition, in certain cases the amino terminal capping group may be a lysine residue or a polylysine peptide, where the polylysine peptide consists of two, three, or four lysine residues, which can prevent cyclization, crosslinking, or polymerization of the peptide compound. Alternatively, longer polylysine peptides may also be used. Another amino capping group that may be used in the peptides described in the invention is an arginine residue or a polyarginine peptide, where the polyarginine peptide consists of two, three, or four arginine residues, although longer polyarginine peptides may also be used. Alternatively the peptide compounds described herein may also be a peptide containing both lysine and arginine, where the lysine and arginine containing peptide is two, three, or four residue combinations of the two amino acids in any order, although longer peptides that contain lysine and arginine may also be used. Lysine and arginine containing peptides used as amino terminal capping groups in the peptide compounds described herein may be conveniently incorporated into whatever process is used to synthesize the peptide compounds to yield the derived peptide compound containing the amino terminal capping group.

In another embodiment of the invention, the peptides may contain a carboxy terminal capping group. The primary purpose of this group is to prevent intramolecular cyclization or inactivating intermolecular crosslinking or polymerization. Furthermore, a carboxy terminal capping group may provide additional benefits to the peptide, such as enhanced efficacy, reduced side effects, enhanced antioxidative activity, and/or other desirable biochemical properties. An example of such a useful carboxy terminal capping group is a primary or secondary amine in an amide linkage to the carboxy terminal amino acid residue. Such amines may be added to the Q-carboxyl group of the carboxy terminal amino acid of the peptide using standard amidation chemistry. In another aspect of the invention, the peptide can be modified by any known modification known to one of ordinary skill in the art. In certain aspects, the peptides may be used as peptidomimetics.

In one aspect, the peptide of the invention can be pegylated. Pegylation, can delay the elimination of the peptides from the circulation by a variety of mechanisms. Pegylation inhibits degradation by proteolytic enzymes and, by increasing the apparent molecular size, reduces the rate of renal filtration. Accordingly, PEG-based modifications are useful to prolong circulation time and bioavailability of the peptides. In one embodiment, the peptide of the invention is pegylated with linear PEG molecules. In another embodiment, the peptide is pegylated with branched PEG molecules. The invention further provides amino-, carboxy- and side-chain pegylated peptides. The PEG moiety can be a PEG molecule with a molecular weight greater than 5 kDa. For example the molecular weight can be between 5 kDa and 100 kDa (e.g., 5, 10, 15, 20, 25, 30, 35, 40, 50, 55, 60, 65, 70, 75, 80, 85, 90, 95 or 100 kDa), and more preferably a molecular weight of between 10 kDa and 50 kDa (e.g., 10, 12, 14, 16, 18, 20, 22, 24, 26, 28, 30, 32, 34, 36, 38, 40, 42, 44, 46, 48, 50 kDa). Methods for synthesis of pegylated peptides are well known in the art.

The invention further provides a peptide with a detectable marker attached thereto. In one embodiment, the detectable marker is attached at the C-terminus of the peptide. In another embodiment, the detectable label is attached to the N-terminus. A detectable marker can be a chemical label such as but no limited to radioactive isotopes, fluorescent groups, chemiluminescent label, colorimetric label, an enzymatic marker, and affinity moieties such as biotin that facilitate detection of the labeled peptide. The invention also provides dye-labeled peptides such as but not limited to fluoresceins, rhodamine conjugates. Other chemical labels and methods for attaching chemical labels to peptides are well-known in the art.

Considering that viruses undergo mutagenic changes in time, a person skilled in the art understands that the inventive peptide sequence may contain amino acid changes at positions that are shown to be subject to natural variation. Such changes are contemplated by the invention as long as these changes do not abolish or decrease the immunosuppressive activity of the peptide.

In one embodiment of the present invention, conservative amino acid substitution in the sequence of the peptides may be performed. Amino acid substitution may be performed insofar as the exchange of amino acid residues occurs from within one of the following groups of residues: Group 1, representing the small aliphatic side chains and hydroxyl group including Ala, Gly, Ser, Thr, and Pro; Group 2, representing OH and SH side chains including Cys, Ser, Thr and Tyr; Group 3, representing residues which have carboxyl containing side chains such as Glu, Asp, Asn and Gln; Group 4, representing basic side chains including His, Arg and Lys; Group 5, representing hydrophobic side chains including Ile, Val, Leu, Phe and Met; and Group 6, representing aromatic side chains including Phe, Trp, Tyr and His. In another embodiment the peptide may have other amino acid substitutions or modifications that do not abrogate the immunosuppressive function of the peptide.

Modifications and substitutions are not limited to replacement of amino acids. One skilled in the art will recognize the need to introduce by means of deletion, replacement, or addition other modifications that provide a peptide with immunosuppressive activity. Examples of such other modifications include incorporation of rare amino acids, dextra (D)-amino acids, glycosylation sites, cytosine for specific disulfide bridge formation. The modified peptides can be chemically synthesized by methods known in the art, or the isolated nucleic acid sequence can be expressed, after site-directed mutagenesis if necessary, in bacteria, yeast, baculovirus, tissue culture and so on.

In one aspect the invention also provides a peptide existing as a monomer. The composition comprises the free peptide or a peptide fragment coupled to a carrier molecule. The peptide may also be used as a conjugate of at least one peptide or a peptide fragment bound to a carrier. The carrier can provide solid phase support for the peptide of the invention. The carrier may be a biological carrier such as a glycosaminoglycan, a proteoglycan, or albumin, or it may be a synthetic polymer such as a polyalkyleneglycol or a synthetic chromatography support. Other carriers include ovalbumin and human serum albumin, other proteins, and polyethylene glycol.

Still other carriers that may be used in the pharmaceutical compositions of this invention include ion exchangers, alumina, aluminum stearate, lecithin, non-albumin serum proteins, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, and wool fat. Such modifications may both increase the apparent affinity and change the stability of a peptide. Although the number of peptide fragments bound to each carrier can vary, typically about 4 to 8 peptide fragments per carrier molecule are bound under standard coupling conditions.

In another aspect of the invention, peptidomimetic compounds, may be designed based upon the amino acid sequences of the peptides of the invention. In one aspect, the peptidomimetic compounds comprise synthetic compounds with conformation substantially similar to the conformation of the peptides of the invention. The structure of the peptidomimetic compound can be similar to the secondary or tertiary structure of the immunosuppressive peptide. The structural similarity of the peptidomimetic compound to the secondary or tertiary structure of the inventive peptide provides the peptidomimetic compound with the ability to suppress an immune response in a manner qualitatively identical to immunosuppression due to the inventive peptide or the peptide fragment from which the peptidomimetic was derived. Furthermore, the peptidomimetic compounds might have additional characteristics that enhance their therapeutic utility, such as increased cell permeability and a prolonged biological half-life.

The backbone of the peptidomimetics are partially or completely non-peptide, but their side groups are identical to the side groups of the amino acid residues that occur in the peptide on which the peptidomimetics are based. Several types of chemical bonds, e.g., ester, thioester, thioamide, retroamide, reduced carbonyl, dimethylene and ketomethylene bonds, are known in the art to be generally useful substitutes for peptide bonds in the construction of protease-resistant peptidomimetics.

In another aspect, the invention provides an immunosuppressive peptide that exists as a dimer. In one embodiment, the dimer may comprise a disulfide bond. In another embodiment, the immunosuppressive peptide dimer may comprise short heterologous sequence fragments that may facilitate dimer formation. In yet another embodiment, two immunosuppressive monomers can be coupled to a carrier molecule in a manner that provides a dimer formation. The peptide can be any of the following: pegylated, labeled with a detectable marker, linked to a carrier that can be a solid phase substrate or conjugated at a free amine group with a polyalkylene glycol. In one embodiment, the immunosuppressive monomers in the dimer may have identical amino acid sequence. In another embodiment, the immunosuppressive monomers in the dimer may have different amino acid sequences.

The peptides in the current invention can be synthesized using standard methods known in the art. Direct synthesis of the peptides of the invention may be accomplished using solid-phase peptide synthesis, solution-phase synthesis or other conventional means. For example, in solid-phase synthesis, a suitably protected amino acid residue is attached through its carboxyl group to an insoluble polymeric support, such as a cross-linked polystyrene or polyamide resin. In our context, a protected amino acid refers to the presence of protecting groups on both the amino group of the amino acid, as well as on any side chain functional groups. The benefit of side chain protecting groups are that they are generally stable to the solvents, reagents, and reaction conditions used throughout the synthesis and are removable without affecting the final peptide product. Typically, stepwise synthesis of the polypeptide is carried out by the removal of the N-protecting group from the initial carboxy terminal and coupling it to the next amino acid in the sequence of the polypeptide. The carboxyl group of the incoming amino acid can be activated to react with the N-terminus of the bound amino acid by formation into a reactive group such as formation into a carbodiimide, a symmetric acid anhydride, or an active ester group such as hydroxybenzotriazole or pentafluorophenyl esters. The solid-phase peptide synthesis methods include both the BOC and FMOC methods, which utilizes tert-butyloxycarbonyl, and 9-fluorenylmethloxycarbonyl as the α-amino protecting groups, respectively, both well-known by those of skill in the art (Sambrook et al., Molecular Cloning: A Laboratory Manual, 2nd edition, Cold Spring Harbor Press, Cold Spring Harbor, N.Y.; Ausubel et al., Current Protocols in Molecular Biology, John Wiley and Sons, New York, 1995).

In another embodiment of the invention, the peptides may also be prepared and stored in a salt form. Various salt forms of the peptides may also be formed or interchanged by any of the various methods known in the art, e.g., by using various ion exchange chromatography methods. Cationic counter ions that may be used in the compositions include, but are not limited to, amines, such as ammonium ions, metal ions, especially monovalent, divalent, or trivalent ions of alkali metals including sodium, potassium, lithium, cesium; alkaline earth metals including calcium, magnesium, barium; transition metals such as iron, manganese, zinc, cadmium, molybdenum; other metals like aluminum; and possible combinations of these. Anionic counter ions that may be used in the compositions described below include chloride, fluoride, acetate, trifluoroacetate, phosphate, sulfate, carbonate, citrate, ascorbate, sorbate, glutarate, ketoglutarate, and possible combinations of these. Trifluoroacetate salts of peptide compounds described here are typically formed during purification in trifluoroacetic acid buffers using high-performance liquid chromatography (HPLC). Although usually not suited for in vivo use, trifluoroacetate salt forms of the peptides described in this invention may be conveniently used in various in vitro cell culture studies, assays or tests of activity or efficacy of a peptide compound of interest. The peptide may then be converted from the trifluoroacetate salt by ion exchange methods or synthesized as a salt form that is acceptable for pharmaceutical or dietary supplement compositions.

In another embodiment, the inventive peptides can be prepared using recombinant DNA technology methods wherein an expression vector comprises nucleic acid sequence encoding any of the peptides of SEQ ID NOS:1 to 85, or any other peptide of the invention, wherein the nucleic acid sequence is operably linked to a promoter. The expression vector can be delivered to, for example but not limited to, by methods of transformation, transfection, etc, a suitable host cell that allows expression of the peptide. Host cells comprising the expression vector are cultured under appropriate conditions and the peptide is expressed. In one embodiment the host cell is a mammalian cell, including human cell. In another embodiment, the host cell is bacterial, fungal or insect cell. In one embodiment the peptide is recovered from the culture wherein the recovery may include a step that leads to the purification of the peptide. Preparation of the inventive peptides by recombinant technology can be advantageous if the peptides can be post-translationally modified. Further still, a combination of synthesis and recombinant DNA techniques can be employed to produce the amide and ester derivatives of this invention, as well as to produce fragments of the desired polypeptide which are then assembled by methods well known to those skilled in the art.

Expression vectors suitable for nucleic acid sequence delivery and peptide expression in human cells are known in the art. Non-limiting examples are plasmid, viral or bacterial vectors.

Peptides according to the invention may also be prepared commercially by companies providing peptide synthesis as a service (e.g., BACHEM Bioscience, Inc., King of Prussia, Pa.; AnaSpec, Inc., San Jose, Calif.). Automated peptide synthesis machines, such as manufactured by Perkin-Elmer Applied Biosystems, also are available

The peptides useful in the methods of the present invention are purified once they have been isolated or synthesized by either chemical or recombinant techniques. Standard methods for purification purposes can be used, including reversed-phase high-pressure liquid chromatography (HPLC) using an alkylated silica column such as C⁴-, C₂- or C₁₈-silica. In this method, a gradient mobile phase of increasing organic content is generally used to achieve purification, for example, acetonitrile in an aqueous buffer, usually containing a small amount of trifluoroacetic acid. Alternatively, ion-exchange chromatography can also be used to separate peptide compounds based on their charge. The degree of purity of the peptide compound may be diagnosed by the number of peaks identified by HPLC. A level of peptide purity useful in the invention can result in a single peak on the HPLC chromatogram. In one embodiment, the peptide of interest is at least 94.99% of the input material on the HPLC column. In another embodiment, the peptide of interest is at least 96.99% of the input material on the HPLC column. In one embodiment, the peptide of interest is between 97% and 99.5% of the input material on the HPLC column.

In one aspect the invention provides isolated, including but not limited to synthesized filovirus peptides, for example having an amino acid sequence motif NRXX(X1)DXL(X2)X(R)XXXXC or NRXX(X1)DXL(X2)X(R)XXXX as provided herein, which peptides can lead to decreased production of pro-inflammatory molecules (IL-2 and IL-12p40) and increased production of IL-10 in human peripheral mononuclear cells results. In one aspect, the present invention demonstrates the immunosuppressive activity of the inventive peptide derived from filoviral glycoprotein polypeptide. Provided are also insight into mechanisms of pathogenesis in filovirus infection and disclose potential therapeutic targets for these frequently fatal infections. The invention also provides use of theses peptide sequences for modulation of the immune response in a wide variety of disorders where inflammation is important in pathogenesis. The present invention further provides methods for the isolation and production of the inventive peptides with therapeutic, including but not limited to immunosuppressive, activity. The invention also provides methods for use of the inventive peptides in treating autoimmune disorders.

In one aspect of the invention, a method to determine whether or not a peptide exhibits an immunosuppressive activity is the lymphoproliferation assay. In the lymphoproliferation assay, PBMCs are cultured with mitogens (e.g. PHA, ConA) in the presence and absence of a peptide of the invention. Following a prescribed incubation period of 72 hours, ³H-thymidine is added to the co-culture for an additional 18-24 hours. With each new round of cellular replication, ³H-thymidine is incorporated into the newly synthesized DNA of the daughter cell and correlates directly with the proliferation or lack of proliferation of the PBMC in culture. Incorporated ³H-thymidine is determined in the art by counting the number of β-particles emitted per minute from the radioactive thymidine using a beta-counter. Immunosuppressive activity of the peptide is determined by comparing the counts-per-minute of the PBMC treated with mitogen alone, versus PBMC treated with mitogen plus a peptide of the invention (J. Coligan, A. Kruisbeek D. Margulies and E. Shevach, Current Protocols in Immunology, Section 7.10, John Wiley & Sons, 1997).

Another method to determine whether or not a peptide exhibits an immunosuppressive activity is the cytotoxic T lymphocyte assay. This assay is a quantitative measure of the ability of activated T lymphocytes to specifically kill target cells expressing a known antigen. These activated cells develop during in vivo exposure or by in vitro sensitization to a specific antigen (e.g. keyhole limpet hemocyanin, KLH). To test the immunosuppressive ability of a peptide of the invention, a peptide is added to the activated T lymphocytes and cultured for a period of 6 days. The CTL assay consists of, on the seventh day, culturing sensitized lymphocytes with a fixed number of target cells (expressing the sensitizing agent) that have been prelabeled with ⁵¹Cr. To prelabel the target cells, the cells are incubated with radioactive ⁵¹Cr which is taken up and reversibly and binds to cytosolic proteins. When these target cells are incubated with sensitized lymphocytes, the target cells are killed and the ⁵¹Cr is released and detected by a radioactive counter. The amount of ⁵¹Cr detected correlates directly with CTL activity. Immunosuppressive activity of the peptide is determined by comparing the amount of ⁵¹Cr released by the activated T lymphocytes alone compared to the activated T lymphocytes cultured with a peptide of the invention (J. Coligan, A. Kruisbeek D. Margulies and E. Shevach, Current Protocols in Immunology, Section 3.11, John Wiley & Sons, 1997).

The expression or downregulation of certain activation markers on PBMC can also be used to detect the immunosuppressive ability of a peptide of the invention. For example, PBMC can be activated by incubation with a mitogen (e.g. PHA, ConA) and cultured for 5-24 hours in the presence or absence of a peptide of the invention. Cells are then labeled with fluorescent-conjugated antibodies specific for activation markers known in the art such as CD3, CD25, CD28, CD69, ICAM-1, LFA-1 and CTLA-4. The expression of these markers is detected by measuring mean fluorescence using flow cytometry, commonly known in the art. The reduction of CD25, CD28, CD69, ICAM-1 and LFA-1 or an increase in CTLA-4 expression indicates a suppression of the activation state of PBMC. The immunosuppressive ability of a peptide from the invention can be determined by comparing expression levels of these markers in PBMC treated with a peptide of the invention, versus PBMC not treated with a peptide of the invention (J. Coligan, A. Kruisbeek D. Margulies and E. Shevach, Current Protocols in Immunology, Section, 5.3-5.4, John Wiley & Sons, 1997).

Similarly, measuring intracellular or secreted cytokines following stimulation of PBMC with a mitogen in the presence or absence of a peptide of the invention is another method to determine the immunosuppressive capability of the peptide. After activation of PBMC, as described above, the polymerase chain reaction (PCR) or flow cytometry can be used to detect intracellular cytokines. As commonly known in the art, following a prescribed incubation period, (e.g. 5-24 hours) mRNA is isolated from the PBMC and primers specific for activation cytokines (IL2, IFN-γ, TNF-α) and suppressive cytokines (IL4, IL10) are used to detect and amplify the mRNA. This semi-quantitative measure of RNA indicates which cytokines have been activated and correlates with the ability of a peptide of the invention to suppress or not suppress an immune response. In another embodiment, the cells from the above mentioned culture are permeabilized (e.g. with saponin) and fluorescent-conjugated antibodies specific for activation cytokines (IL2, IFN-γ, TNF-α) and suppressive cytokines (IL4, IL10) are used to detect their presence by flow cytometry methods known in the art. In another embodiment of the invention, the immunosuppressive activity of a peptide can be determined by measuring cytokine production and secretion following in vitro treatment of peripheral blood mononuclear cells with a test peptide as previously described. Following stimulation of the PBMC, supernatant is collected and cytokine levels are measured using standard methods in the art such as ELISA or chemiluminescence (J. Coligan, A. Kruisbeek D. Margulies and E. Shevach, Current Protocols in Immunology, Sections 5.5, 10.3, John Wiley & Sons, 1997).

Yet another method to determine whether or not a peptide exhibits an immunosuppressive activity is the delayed type hypersensitivity (DTH) model or vaccine studies, well known in the art. For example, a mouse DTH/vaccine model using KLH or ovalbumin (OVA) as the immunogen may be used to detect the immunosuppressive activity of a peptide. Immunizing mice with KLH, for example, in the presence and absence of a peptide of the invention, re-immunizing the mice 7 days later and measuring footpad swelling after 48 hours may demonstrate the immunosuppressive activity of the peptides of the current invention. In an uncompromised immune setting, the footpad will swell to a significantly larger size as known in the art. If the immune system is suppressed, the footpad will swell significantly less or not at all. Analyzing which result was induced in the presence of a peptide of the invention will indicate if it is capable of suppressing an immune response (J. Coligan, A. Kruisbeek D. Margulies and E. Shevach, Current Protocols in Immunology, Section 4.5, John Wiley & Sons, 1997).

Another method to measure the immunosuppressive ability of a peptide of the invention is to test the peptide in an experimental autoimmune encephalitis (EAE) model or a systemic lupus erythematosus model (SLE). Briefly, EAE is a demyelinating disease of the central nervous system that resembles Multiple Sclerosis (MS). The disease appears in exacerbations and remissions and is characterized by loss of nerve conduction and chronic progression of disability. Macrophages and T-lymphocytes mediate the destruction of the myelin sheath around the nerves leading to improper nerve conduction. Mouse models for EAE are well known in the art (J. Coligan, A. Kruisbeek D. Margulies and E. Shevach, Current Protocols in Immunology, Section 15.1, John Wiley & Sons, 1997), and when mice are immunized with a given amount of peptide from the invention, the autoimmune reactivity may be reduced and eliminated, compared to untreated EAE mice, demonstrating the immunosuppressive capacity of a peptide of the invention.

SLE is a multiphenotypic autoimmune disease impacting several organ systems of the body. The hallmark of SLE is the production of anti-double-stranded DNA autoantibodies and the deposition of immune complexes in target tissues such as the kidney, skin, and brain. Additional phenotypic traits are the presence of arthritis, anemia, central nervous system involvement, and a variety of autoantibodies. Animal models for SLE are also well known in the art (J. Coligan, A. Kruisbeek D. Margulies and E. Shevach, Current Protocols in Immunology, Section 15.20, John Wiley & Sons, 1997) and represent another method to evaluate the immunosuppressive quality of the invention. For example, immunizing mice with a peptide from the invention may reduce or ablate autoimmune reactivity in the mice exposed to a peptide from the invention compared to control SLE mice.

In another embodiment, the invention provides an antibody, or a portion thereof, such as a Fab fragment, with a specificity for a peptide of the invention. These antibodies may be monoclonal, polyclonal, IgG, IgM or IgA. The antibodies may be chimeric or humanized goat, rabbit, mouse, rat, or monkey anti-peptide, wherein the constant region of the antibody is derived from human genetic sequences making the antibody less immunogenic in a human host. The invention also provides a binding protein with a specificity for a peptide of the invention.

In another aspect of the invention, a peptide of the invention is capable of binding to selected regions of the T cell receptor. T cell clones reactive to the peptides of the invention may be identified by panning a population of isolated T cells with peptides of known sequences. Reactive T cells and the corresponding T cell receptor (TCR) genes coding for the binding motifs may be sequenced and the variable regions of the TCR responsible for binding may be determined (J. Coligan, A. Kruisbeek D. Margulies and E. Shevach, Current Protocols in Immunology, Section 7.3, John Wiley & Sons, 1997).

In yet another embodiment, the invention provides a method for suppressing an immune response of a subject by administering a peptide of the invention in a therapeutically effective amount. The invention can be used as a method to treat a subject suffering from an autoimmune disease or other pro-inflammatory conditions, comprising administering to the subject an effective amount of a peptide of the invention to suppress the subject's immune response and thereby treat the disease or condition. Diseases include, but are not limited to, diabetes mellitus, rheumatoid arthritis, multiple sclerosis, systemic lupus erythematosis, myasthenia gravis, scleroderma, Crohn's disease, ulcerative colitis, Hashimoto's thyroiditis, Graves' disease, Sjogren's syndrome, polyendocrine failure, vitiligo, peripheral neuropathy, graft-versus-host disease, autoimmune polyglandular syndrome type I, acute glomerulonephritis, Addison's disease, adult-onset idiopathic hypoparathyroidism (AOIH), alopecia totalis, amyotrophic lateral sclerosis, ankylosing spondylitis, autoimmune aplastic anemia, autoimmune hemolytic anemia, Behcet's disease, Celiac disease, chronic active hepatitis, CREST syndrome, dermatomyositis, dilated cardiomyopathy, eosinophilia-myalgia syndrome, epidermolisis bullosa acquisita (EBA), giant cell arteritis, Goodpasture's syndrome, Guillain-Barre syndrome, hemochromatosis, Henoch-Schonlein purpura, idiopathic IgA nephropathy, juvenile rheumatoid arthritis, Lambert-Eaton syndrome, linear IgA dermatosis, myocarditis, narcolepsy, necrotizing vasculitis, neonatal lupus syndrome (NLE), nephrotic syndrome, pemphigoid, pemphigus, polymyositis, primary sclerosing cholangitis, psoriasis, rapidly-progressive glomerulonephritis (RPGN), Reiter's syndrome, stiff-man syndrome and thyroiditis.

Alternatively, the present invention provides a method of suppressing the immune response of a subject by co-administering a peptide of the invention with an immunosuppressive agent or compound. The peptide may be co-administered with, but not limited to, cyclosporin, glucocorticoids, prednisone, methotrexate, rapamycin, tacrolimus, mycodphenolate mofetil, sirolimus, monoclonal anti-CD25 antibody, polyclonal anti-lymphocyte antibody, and “humanized” mouse monoclonal antibody. In one embodiment of the invention, the peptides could be administered with lowered doses of the co-administered immunosuppressive agent or compound. The subjects in this embodiment may comprise a human, a primate, a mammal, a fish or any other living organism with an immune system.

In one embodiment, the immunosuppressive peptide of the invention can be delivered in the form of a peptide. In another embodiment, the immunosuppressive peptide of the invention can be delivered by an expression vector comprising a nucleic acid encoding the immunosuppressive peptide.

Administering the peptide of the invention, either in a peptide form or as an expression vector, may be done by a variety of routes or modes. These include, but are not limited to, parenteral, oral, intratracheal, sublingual, pulmonary, topical, rectal, nasal, buccal, sublingual, vaginal, or via an implanted reservoir. Implanted reservoirs may function by mechanical, osmotic, or other means. The term “parenteral”, as used here, includes intravenous, intracranial, intraperitoneal, paravertebral, periarticular, periostal, subcutaneous, intracutancous, intra-arterial, intramuscular, intra-articular, intrasynovial, intrastermal, intrathecal, and intralesional injection or infusion techniques. Such compositions are formulated for parenteral administration, and most for intravenous, intracranial, or intra-arterial administration. Generally, when administration is intravenous or intra-arterial, pharmaceutical compositions may be given as a bolus, as separated doses.

A peptide of the invention may be in the form of a sterile injectable preparation, for example, as a sterile injectable aqueous suspension. This suspension may be formulated according to techniques known in the art using suitable dispersing or suspending agents. The sterile injectable preparation may also be a sterile injectable solution or suspension in a non-toxic parenterally acceptable diluent or solvent, for example, as a solution in 1,3-butanediol. Acceptable solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil.

A peptide of this invention may be orally administered via capsules, tablets, caplets, pills, aqueous suspensions, reconstituted lyophilized preparation, and solutions, or syrups. In the case of tablets for oral use, carriers, including lactose and cornstarch, may be used. Lubricating agents, such as magnesium stearate, are also sometimes added. For oral administration in a capsule form, useful diluents include lactose and dried cornstarch. Capsules, tablets, pills, and caplets may be formulated for delayed or sustained release when long-term expression is required.

Alternatively, when orally aqueous suspensions are to be administered, the peptide is advantageously combined with emulsifying and/or suspending agents. If desired, certain sweetening and/or flavoring and/or coloring agents may be added. In one embodiment the preparation for oral administration provides a peptide of the invention in a mixture that prevents or inhibits hydrolysis of the peptide compound by the digestive system, thereby allowing absorption into the blood stream.

Also, a peptide of this invention may be administered mucosally (e.g. vaginally or rectally). These dosages can be prepared by mixing a peptide of this invention with a suitable non-irritating excipient, which is solid at room temperature but liquid at body temperature and therefore will change states to liquid form in the relevant body space to release the active compound. Examples of these solvents include cocoa butter, beeswax and polyethylene glycols.

Still, for other mucosal sites, such as for nasal or pulmonary delivery, absorption may occur via the mucus membranes of the nose, or inhalation into the lungs. These modes of administration typically require that the composition be provided in the form of a solution, liquid suspension, or powder, which is then mixed with a gas such as air, oxygen or nitrogen, or combinations thereof, so as to generate an aerosol or suspension of droplets or particles. These preparations are carried out according to well-known techniques in the art of pharmaceutical formulation. These preparations may be made as solutions in saline, employing benzyl alcohol or other suitable preservatives, absorption promoters to enhance bioavailability, fluorocarbons, and solubilizing or dispersing agents known in the art.

In yet another embodiment of the current invention, a peptide of the invention can be used to identify a therapeutic agent. A peptide of the invention may be used to screen for therapeutic agents such as monoclonal or polyclonal antibodies, binding proteins, biologics, chemical compounds or other panels of compounds to identify possible therapeutic agents which would modulate the immunosuppressive function of the peptide and affect the associated disease phenotype. As such, a peptide of the invention may be used to identify therapeutic agents that may inhibit the immunosuppressive activity of the peptide. Such agents can be administered as a treatment during the pathogenic phase of a filovirus infection. Identified therapeutic agents may be tested for specificity and binding activity, and constitute the basis for pharmacological development. In another aspect, these identified therapeutic agents may be used to abrogate the immunosuppression associated with in vivo expression of the peptides during Ebola or Marburg infections.

Another aspect of the invention provides antibodies that bind to the immunosuppressive peptide. In one embodiment, the antibodies can neutralize the immunosuppressive activity of the peptide. In one embodiment, antibodies that specifically bind to the immunosuppressive peptide, as a monomer or dimer, may be used in a therapeutic composition to treat filoviral infection. The anti-immunosuppressive peptide antibodies can be monoclonal or polyclonal. Methods for making polyclonal and monoclonal antibodies are well known in the art. The antibodies can be chimeric, i.e. a combination of sequences of more than one species. The antibodies can be fully-human or humanized Abs. Humanized antibodies contain complementarity determining regions that are derived from non-human species immunoglobulin, while the rest of the antibody molecule is derived from human immunoglobulin. Fully-human or humanized antibodies avoid certain problems of antibodies that possess non-human regions which may trigger host immune response leading to rapid antibody clearance. In one embodiment, antibodies can be produced by immunizing a non-human animal with the immunosuppressive peptide as a monomer or a dimer. The immunogenic composition may comprise other components that can increase the antigenicity of the immunosuppressive peptide. In one embodiment the non-human animal is a transgenic mouse model, for e.g., the HuMAb-Mouse™ or the Xenomouse®, which can produce human antibodies. Neutralizing antibodies against the immunosuppressive peptide and the cells producing such antibodies can be identified and isolated by methods know in the art.

In another aspect, the invention provides a method for treatment of filoviral infection by administering therapeutic agents that can inactivate the immunosuppressive peptide and inhibit the peptide immunosuppressive function. In one embodiment, the therapeutic agent is an antibody that binds to the immunosuppressive peptide. In another embodiment, the therapeutic agent can be a compound that binds to the immunosuppressive peptide. Compositions that comprise these therapeutic agents can be useful for the treatment of filoviral infections. Therapeutic agents that bind to the immunosuppressive peptide can be administered in combination with other agents considered useful in the treatment of filoviral infections.

This invention is illustrated in the Example sections that follow. These sections are set forth to aid in an understanding of the invention but are not intended to, and should not be construed to, limit in any way the invention as set forth in the claims which follow thereafter.

### Examples

Design of the Synthetic Peptides.

Filoviral 17-mer peptides corresponding to the immunosuppressive domain were synthesized. The identification of the region with strong secondary structure similarity to the retrovirus glycoprotein was done using the program 3D-PSSM (22). ZEBOV peptide has amino acid sequence ILNRKAIDFLLQRWGGT (SEQ ID NO: 1). ZEBOV peptide and SEBOV peptide (SEQ ID NO: 3) differ by one residue at position 12, where ZEBOV peptide has glutamine; while SEBOV peptide has arginine); both have isoleucine at position 1. REBOV peptide (SEQ ID NO: 4) differs from ZEBOV peptide only in the presence of leucine at position 1.

Cell Culture.

Human PBMC were isolated from heparinized venous blood of healthy volunteers by density gradient centrifugation over Ficoll-Hypaque (Amersham Biosciences). Monkey PBMC were separated from heparin-treated peripheral blood collected from healthy adult Rhesus (Macaca mulatta) macaques using a similar procedure. Human PBMC were suspended at 10⁶/ml in RPMI 1640 supplemented with 10% FBS (Irvine Scientific) and cultured in the presence of soluble anti-human CD28 at 21 g/ml on plates coated with anti-human CD3 antibody at 101 g/ml (anti-CD3/CD28) alone (23); anti-CD3/CD28 and inactivated ZEBOV (inactZEBOV; equivalent of 25 infectious units per cell prior to y-irradiation using 5×10⁶ rads); or anti-CD3/CD28 and filoviral peptides at 401M concentration. Cells were incubated at 37° C. in 5% CO₂ for 12 or 48 hours prior to analysis. Conditions were similar for experiments with rhesus PBMC except that cells were activated by culture on plates coated with anti-human CDS epsilon antibody (anti-CD30, Clone: SP34, cross-reactive with rhesus CD3, (24)) at 101 g/ml.

Cell Surface Phenotype.

All monoclonal antibodies (mAbs) used in FACS analyses were generated using human antigens; some were human-specific (Caltag): anti-CD4-APC (Clone: S3.5, Isotype: Mouse IgG₂ₐ), anti-CD8-APC (Clone: 3B5, Isotype: Mouse Ig₂ₐ), anti-CD25-FITC (Clone: CD25-3G10, Isotype: Mouse IgGi), anti-CD4-PE (Clone: S3.5, Isotype: Mouse IgG₂ₐ) and anti-CD69-FITC (Clone: CH/4, Isotype: Mouse IgG₂ₐ); others were cross reactive with macaque (25, 26): anti-CD4-PE (Clone: L200, Isotype: Mouse IgG₁κ), anti-CD8-APC-CY7 (Clone: RPA-T8, Isotype: Mouse I_{g}G₁κ) and anti-CD69-FITC (Clone: FN50, Isotype: Mouse I_{g}G₁κ) (BD Pharmingen). At 12 or 48 hours, PBMC were stained for surface expression of CD4, CD8, CD25, and CD69 using the relevant mAbs. Cells were washed twice with RPMI 1640 medium supplemented with 0.5% FBS (wash medium). 1×10⁶ cells were then incubated with fluorochrome-tagged primary antibody in a total volume of 0.1 ml for 30 min at 4° C. Cells were subsequently washed twice with 2 ml of wash medium to remove any unbound antibody and fixed in 0.5 ml of 1% paraformaldehyde solution. Cells were then analyzed by multicolor flow cytometry on a LSRII Analyzer (Becton Dickinson). Data was obtained using FACS DiVa acquisition software (Becton Dickinson), and analyzed using FlowJo6.1 (Tree Star) after appropriate gating to exclude dead cells and debris based on forward scatter and side scatter. Fluorescent markers used were APC (allophycocyanin), FITC (fluorescein isothiocyanate), and PE (phycoerythrin) and APC-CY7 (allophycocyanin-cyanine 7).

5-Bromo-2-Deoxyuridine (BrdU) Labeling and Cell-Cycle Analysis.

Intracellular BrdU was measured using a commercial assay (BrdU Flow Kit, BD Biosciences). Human PBMC were activated with anti-CD3/CD28 in the absence or presence of filoviral peptides for 48 hours. Three hours prior to harvest, 101M of BrdU was added to each well. Cells were resuspended in 5011 of staining buffer (PBS+3.0% FBS). Fluorescent antibodies specific for detection of CD4 and CD8 were added. Cells were fixed, permeabilized and treated with DNase (30|lg per tube) to expose incorporated BrdU. Intracellular BrdU was stained with anti-BrdU-FITC antibody. Cells were washed and 2011 of 7-Amino-Actinomycin D (7-AAD) solution was added for staining of total DNA. Cells were resuspended in staining buffer and analyzed by flow cytometry.

Apoptosis Assays.

PBMC were stained for surface expression of CD4 and CD8 using the relevant mAbs. Cells were washed twice with PBS and resuspended in 0.1 ml Annexin V binding buffer (BD Biosciences) and incubated with 5 ll of FITC-conjugated Annexin V (BD Biosciences) and 10 ll of propidium iodide (PI) for 15 minutes at room temperature. The cells were immediately analyzed by flow cytometry on a FACSCalibur (Becton Dickinson). Data was obtained using CellQuest acquisition software (Becton Dickinson) and analyzed using FlowJo6.1 (Tree Star). Cells stained with Annexin V-FITC alone and PI alone were used as controls.

Cytokine Assays.

Cell-free supernatants from PBMC cultures were collected and analyzed using the Beadlyte Human 11-Plex Cytokine Detection System (Upstate Biotechnology). The lyophilized mixed standard was resuspended in cell culture medium and serially diluted. Samples or standards were incubated with the 11-Plex cytokine capture bead suspension array in a 96-well filter plate for 2 hours at room temperature. The beads were washed and biotinylated reporter 11-plex antibodies were added for 1.5 hours. Streptavidin-PE was then added to each well. After a 30-minute incubation, the beads were washed and resuspended in assay buffer. The median fluorescence intensity of 100 beads per cytokine was read using a Luminex 100 Instrument (Luminex). Concentrations were interpolated from standard curves.

Statistical Analysis.

All statistical analyses were performed using InStat 3 (GraphPad Software). Data from all FACS assays (cell surface phenotype, BrdU incorporation, cell cycle analysis, apoptosis) were first tested for normal distribution by the Kolmogorov and Smirnov (K-S) test and then analyzed for significance using ANOVA and Dunnett's specialized multiple comparison test. Cytokine assays were analyzed using Kruskal-Wallis non-parametric ANOVA and the Dunn multiple comparison test. Cytokine data were fitted on a sigmoidal dose-response curve.

Determining Effect of 17Mer Peptides on PBMCs.

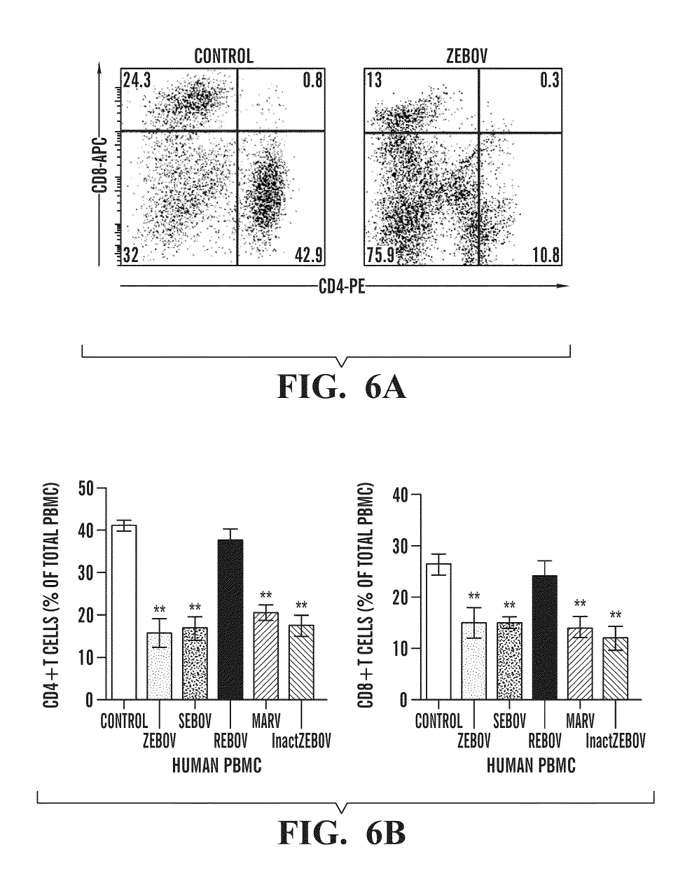

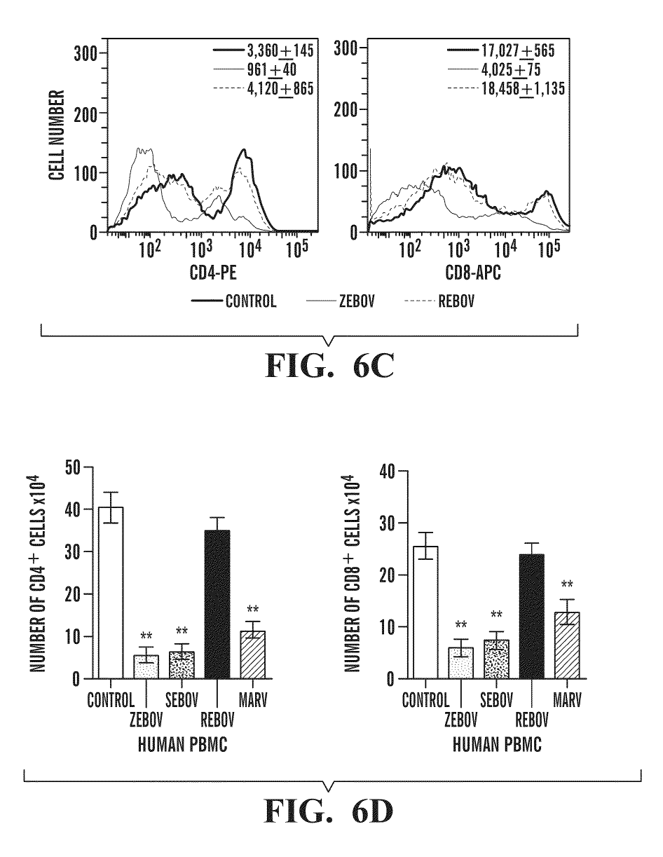

The effect of synthetic 17mer peptides corresponding to a candidate immunosuppressive domain in filoviral glycoproteins were assessed. Human PBMC were exposed for 48 hours to either inactZEBOV or 40 μM filoviral peptides in the presence of anti-CD3/CD28. Flow cytometric analysis revealed a significant decrease in the percentage of cells positive for CD4 and CD8 after treatment with inactZEBOV or ZEBOV, SEBOV or MARV peptide but not REBOV peptide (FIG. 6A, B).

ZEBOV peptide treatment reduced the amount of CD4 and CD8 expressed on the cell surface of human PBMC. Exposure to ZEBOV peptide resulted in a 3.5-fold reduction in the cell surface expression of CD4 and a 4.2-fold reduction in the cell surface expression of CD8 (CD4 expression with ZEBOV peptide, n=5: mean fluorescence intensity value for CD4 expression±standard deviation of the mean [SD], 961±40; CD4 expression without ZEBOV peptide, n=5: 3,360±145; p<0.01; CD8 expression with ZEBOV peptide, n=5, mean fluorescence intensity value for CD8 expression±SD: 4,025±75; CD8 expression without ZEBOV peptide, n=5: 17,027±565; p<0.01; FIG. 6C). A similar decrease in CD4 and CD8 expression was observed on PBMC treated with SEBOV or MARV peptides. No decrease in the expression levels of CD4 or CD8 was observed with REBOV peptide treatment (FIG. 6C).

ZEBOV peptide caused a significant decline in the absolute numbers of both CD4+ and CD8+ T cells. Exposure to ZEBOV peptide resulted in a 7.4-fold decrease in the number of CD4+ T cells and a 4.4-fold decrease in the number of CD8+ T cells (number of CD4+ T cells with ZEBOV peptide, n=5: 5.5±1.8×10⁴; number of CD4+ T cells without ZEBOV peptide, n=5: 40.6±3.7×10⁴; p<0.01; number of CD8+ T cells with ZEBOV peptide, n=5: 5.8±1.6×10⁴; number of CD8+ T cells without ZEBOV peptide, n=5: 25.4+2.6×10⁴; p<0.01; FIG. 6D). A similar decline in absolute T cell numbers was also observed with SEBOV or MARV peptide treatment. REBOV peptide exposure caused no significant depletion of T cells (FIG. 6D).

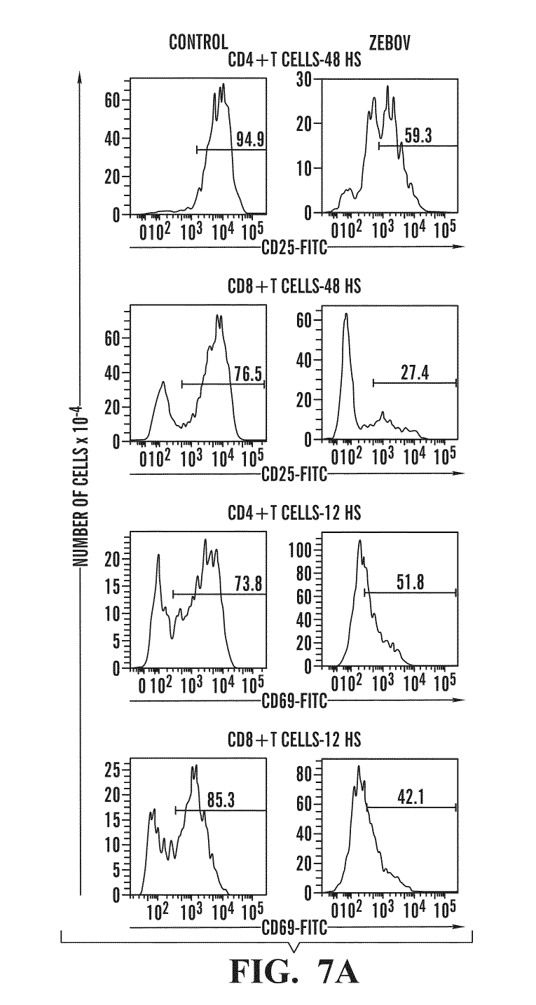

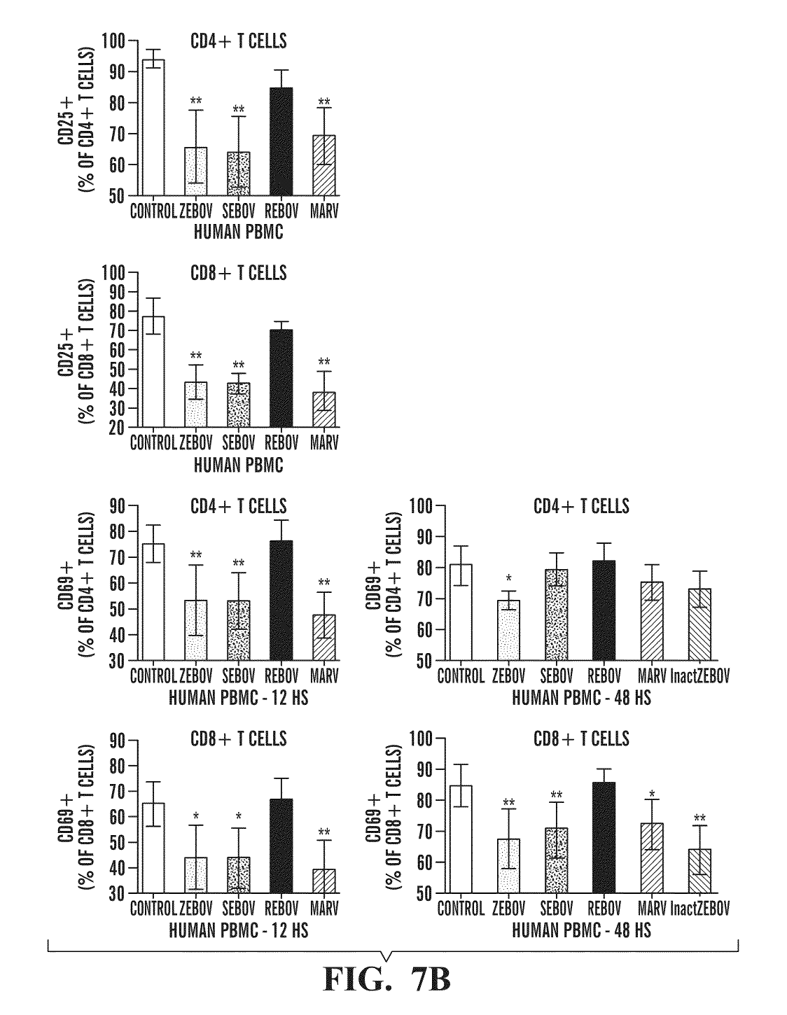

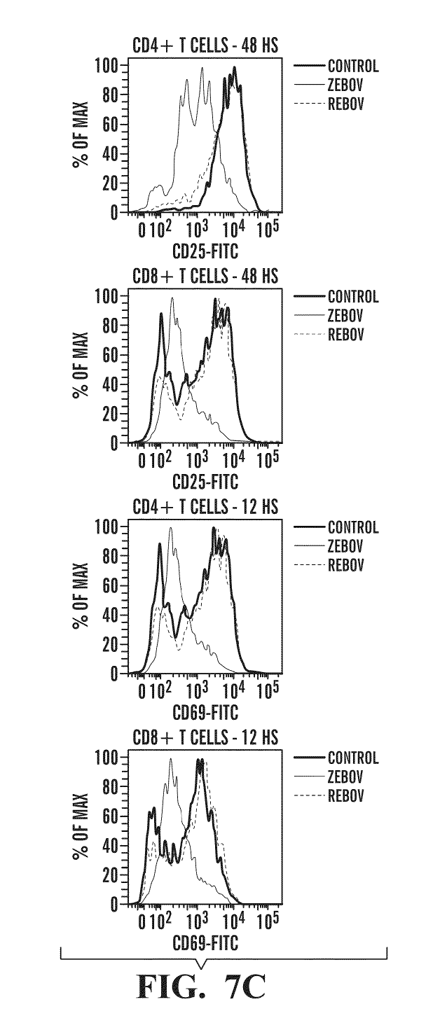

To further characterize the immunosuppression observed with the filoviral peptides, the phenotypic characteristics and status of PBMC exposed to filoviral peptides were evaluated. The interleukin-2 receptor a chain (IL-2R) is an essential component of high-affinity IL-2 receptors. Whereas resting T cells do not express high affinity IL-2R, receptors are rapidly expressed on T cells after activation with antigen or mitogens (27). The interaction of IL-2 with IL-2R triggers proliferation. IL-2R expression (CD25) was measured on human PBMC activated with anti-CD3/CD28 in the presence or absence of filoviral peptides (FIG. 7). ZEBOV peptide treatment resulted in a reduction in the percentages of CD25+ cells in both CD4+ and CD8+ T cell populations (percentage of CD4+ T cells treated with ZEBOV peptide that are CD25+, n=5: 65.9±11.8%; without ZEBOV peptide, n=5: 93.9±3.0%; p<0.01; percentage of CD8+ T cells treated with ZEBOV peptide that are CD25+, n=5: 43.9+9.0%; without ZEBOV peptide, n=5: 77.9±9.2%; p<0.01; FIG. 7A, B). Similar effects on IL-2R expression were obtained in both CD4+ and CD8+ T cells after exposure to SEBOV or MARV peptides. No effect was observed with the nonpathogenic strain, REBOV (FIG. 7B). The mean fluorescent intensity of CD25 expression was also decreased on CD4+ and CD8+ T cells treated with ZEBOV peptide but not REBOV peptide (FIG. 7C).

Lymphocyte activation in response to polyclonal mitogens, antibodies or antigens is characterized by coordinated surface expression of activation/adhesion molecules. CD69 expression was used as a marker for T cell activation (Hara et al., 1986) following exposure to anti-CD3/CD28 in the presence of inactZEBOV or filoviral peptides. Exposure for 48 hours to ZEBOV peptide resulted in a decrease in the percentages of CD69+ cells in both CD4+ and CD8+ T cell populations (percentage of CD4+ T cells treated with ZEBOV that are CD69+, n=5: 69.4±3.4%; without ZEBOV, n=5: 80.8±6.4%; p<0.05; percentage of CD8+ T cells treated with ZEBOV that are CD69+, n=5: 67.9±9.6%; without ZEBOV, n=5: 84.9±6.9%; p<0.05; FIG. 7B). Exposure for 48 hours to SEBOV peptide or MARV peptide, or inactZEBOV resulted in a significant reduction in the percentage of CD69+ CD8+ T cells; a trend toward reduction was observed in CD69+CD4+ T cells that did not achieve statistical significance (FIG. 7B). Exposure for 12 hours to ZEBOV, SEBOV, or MARV peptide resulted in a significant reduction in percentages of both CD69+CD4+ T cells and CD69+CD8+ T cells (FIG. 7A, B). No effect was observed with REBOV peptide at either 12 or 48 hours (FIG. 7B). The mean fluorescent intensity of CD69 expression was also decreased on CD4+ and CD8+ T cells treated for 12 hours with ZEBOV peptide but not REBOV peptide (FIG. 7C).

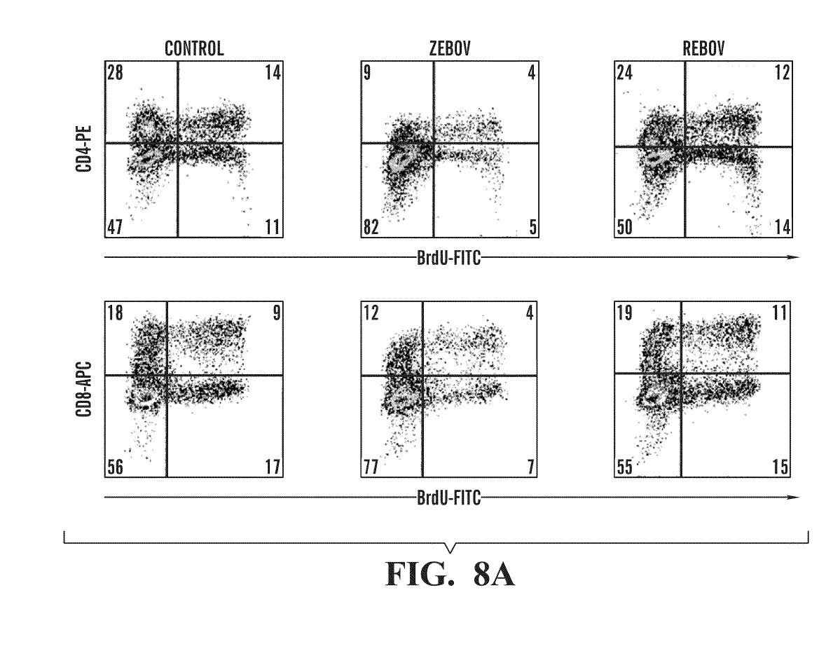

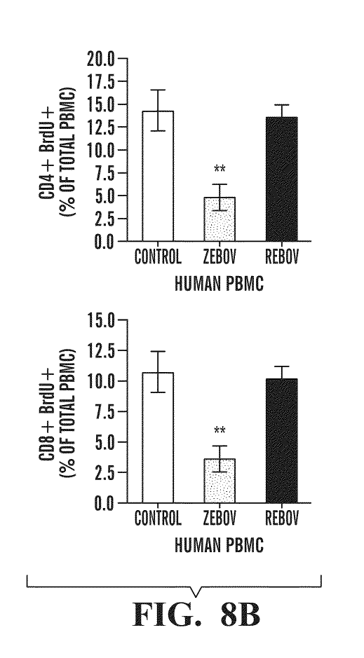

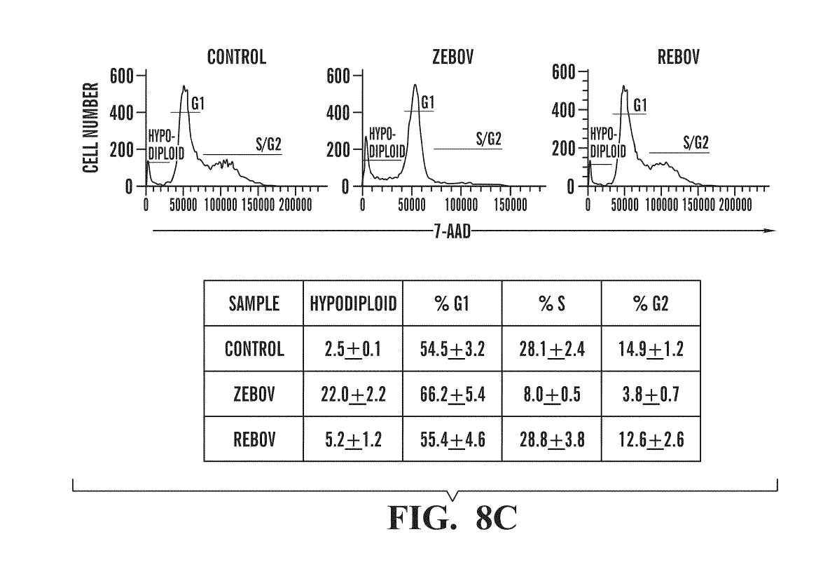

Proliferative responses of T lymphocytes exposed to filoviral peptides were assessed by flow cytometric measurement of BrdU incorporation. Human PBMC were treated with anti-CD3/CD28 in the presence or absence of ZEBOV peptide or REBOV peptide for 48 hours. BrdU was added for the final 3 hours of culture. ZEBOV peptide treatment resulted in decreased BrdU labeling of CD4+ and CD8+ T cells (percentage of BrdU labeled CD4+ cells treated with ZEBOV peptide, n=5: 3.9±0.6%; without ZEBOV peptide, n=5: 14.4±2.3%; p<0.01; percentage of BrdU labeled CD8+ cells treated with ZEBOV peptide, n=5: 4.8±1.8%; without ZEBOV peptide, n=5: 9.4+0.9%; p<0.01; FIG. 8A, B). No significant change in BrdU labeling was observed with REBOV peptide (FIG. 8A, B). Cell-cycle analysis of PBMC treated with ZEBOV peptide and anti-CD3/CD28 revealed an increase in the hypodiploid population together with decreased cell-cycle progression (FIG. 8C). ZEBOV peptide treated PBMC showed an 8.8-fold increase in the number of cells with hypodiploid DNA content (percentage of peptide-treated cells with hypodiploid DNA, n=5: 22.0±2.2%; untreated cells, n=5: 2.5±0.1%; p<0.01) consistent with an induction in apoptosis (29) (FIG. 8C). A 3.5-fold decrease in the percentage of cells in the S phase was observed with ZEBOV peptide treatment (percentage of peptide-treated cells in S phase, n=5: 8.0±0.5%; untreated cells, n=5: 28.1+2.4%; p<0.01) suggesting a decrease in the numbers of actively cycling cells. No change in the cycling pattern was observed with PBMC treated with REBOV peptide (FIG. 8C). These results show that filoviral peptide treatment may reduce the numbers of T cells by depression of proliferative responses, and/or by induction of apoptosis.

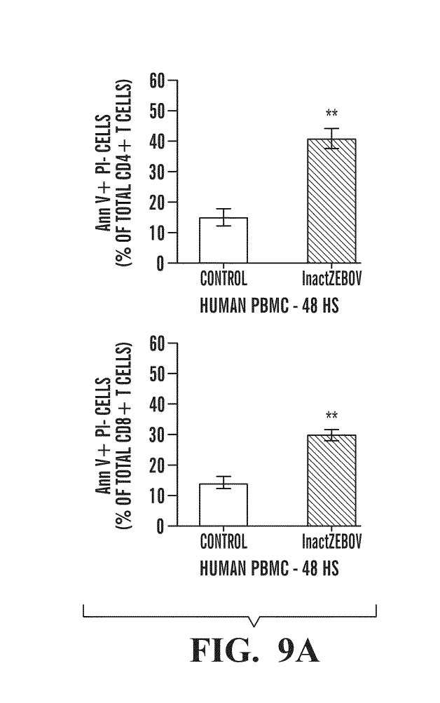

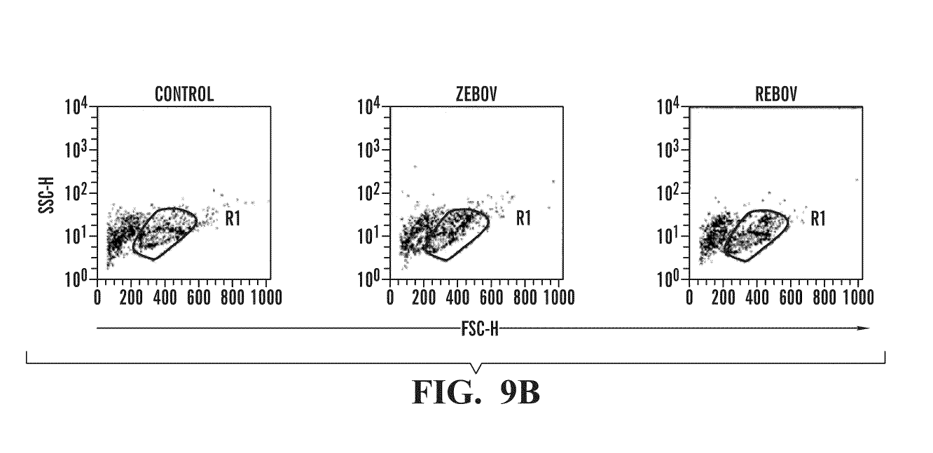

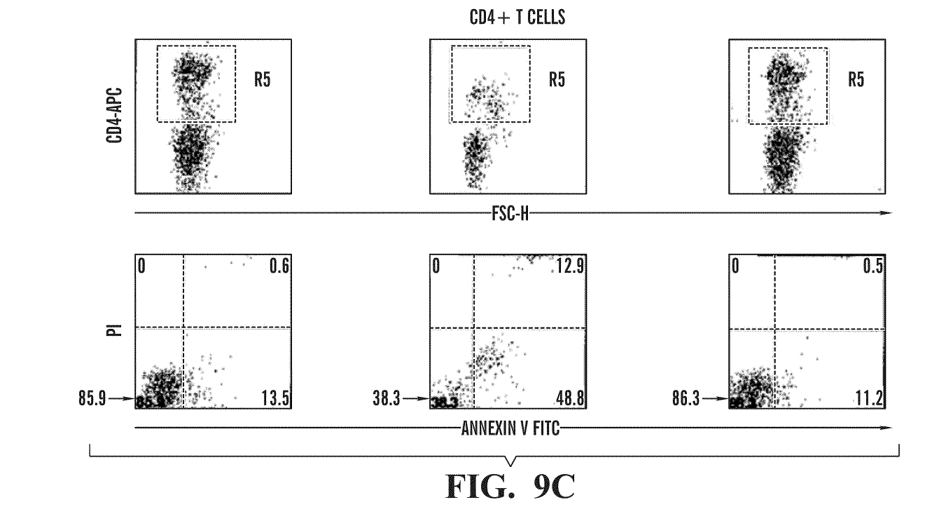

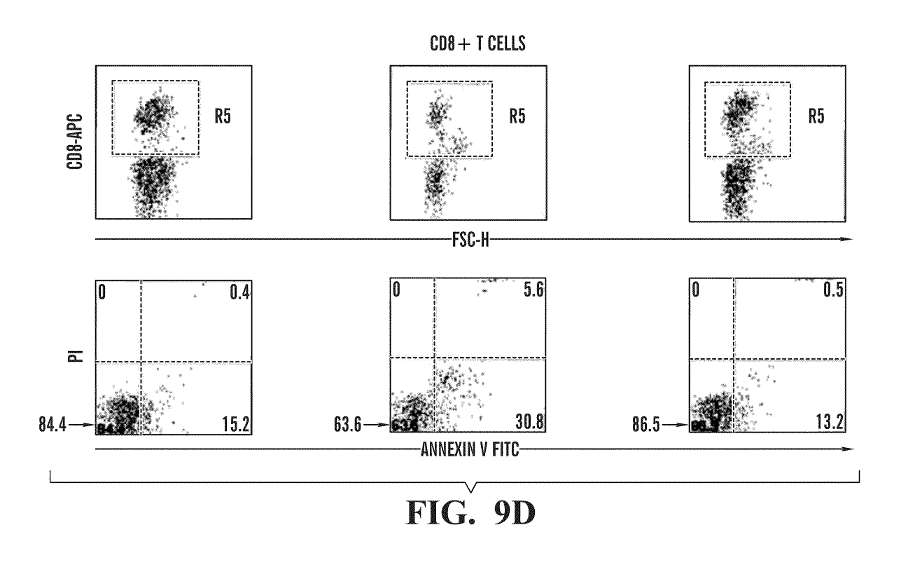

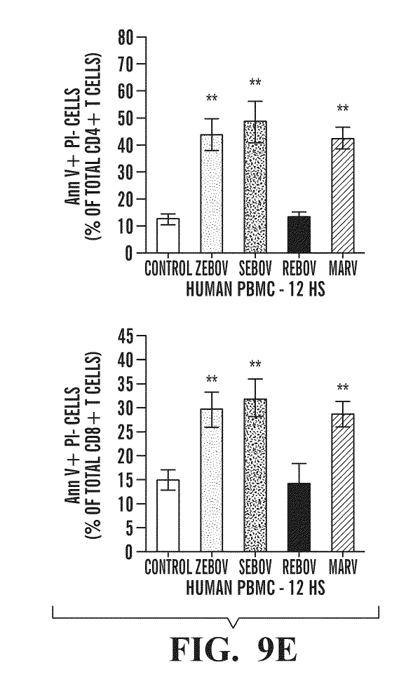

Profound lymphopenia and lymphoid depletion due to apoptosis are characteristic features of fatal filoviral infections (Baize et al., 1999). Apoptosis may be independent of viral replication (Geisbert et al, 2000, Hensley et al, 2002). Treatment of human PBMC with inactZEBOV for 48 hours in the presence of anti-CD3/CD28 resulted in a 2.9-fold increase in apoptotic cells in the CD4+ population and a 2.1-fold increase in the CD8+ population (percentage of Annexin V+ PI− CD4+ exposed to inactZEBOV, n=5: 41.0±3.3%; untreated cells, n=5: 14.3±2.3%, p<0.01; percentage of Annexin V+ PI− CD8+ exposed to inactZEBOV, n=5: 30.1+1.9%; untreated cells, n=5: 14.2±2.1%, p<0.01; FIG. 9A). ZEBOV peptide treatment also resulted in induction of apoptosis in both CD4+ and CD8+ T cells (FIG. 9B, C, D). Human PBMC were exposed to ZEBOV peptide in the presence of anti-CD3/CD28 for 12 hours and subjected to flow cytometric analysis. Viable PBMC were gated according to forward scatter (FSC) and side scatter (SSC) profile (R1 gate, FIG. 9B). Live (R1) cells were further gated on CD4+ cells according to CD4 expression and FSC and on CD8+ cells according to CD8 expression and FSC (R5 gate; FIG. 9C, D). The percentages of apoptotic cells in CD4+ and CD8+ T cell populations were determined by Annexin V/PI staining. Cells positive for Annexin V and negative for P1 were considered apoptotic (FIG. 9C, D). ZEBOV peptide treatment resulted in a 3.6-fold increase in apoptotic CD4+ cells and a 2.0-fold increase in apoptotic CD8+ cells (percentage of Annexin V+ PI-CD4+ treated with ZEBOV peptide, n=5: 43.6±5.8%; untreated cells, n=5: 12.2±1.9%; p<0.01; percentage of Annexin V+ PI− CD8+ treated with ZEBOV peptide, n=5: 29.7±3.7%; untreated cells, n=5: 14.9±2.1%; p<0.01; FIG. 9C, D). Effects were similar with human PBMC exposed to SEBOV or MARV peptides (FIG. 9E). No significant induction of apoptosis was observed following treatment with REBOV peptide (FIG. 9B, C, D, E). Taken together, these data implicate apoptosis in T cell depletion following filoviral peptide exposure, and are consistent with the observation that whereas ZEBOV, SEBOV, and MARV are pathogenic for humans, REBOV is not.

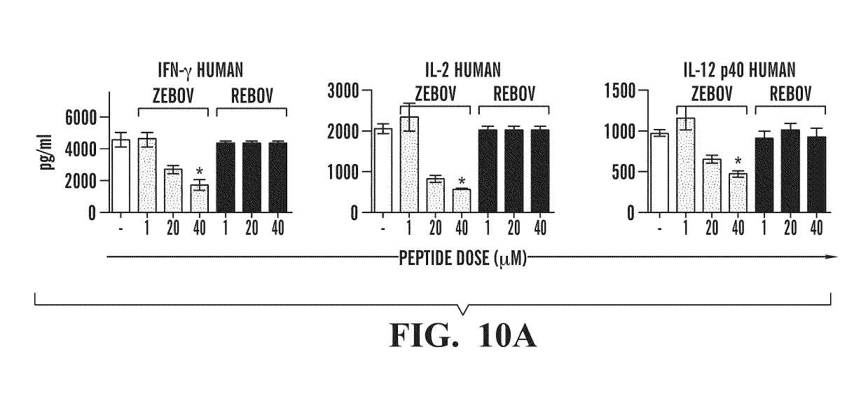

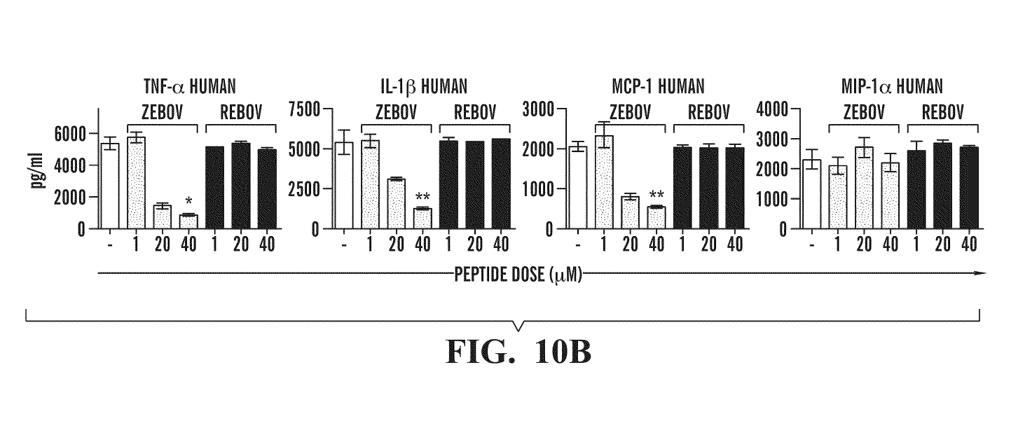

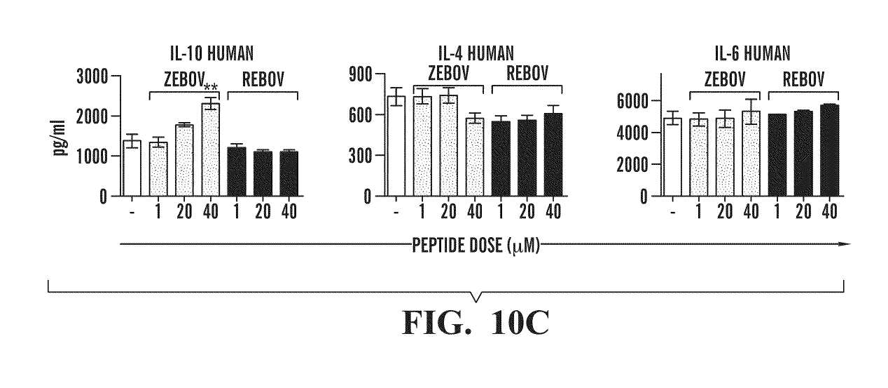

Cytokines and chemokines play important roles in immunopathological processes and normal immune response. In addition, there is evidence for the involvement of inflammatory mediators in the pathogenesis of EBOV infection from previous studies wherein infected individuals had elevated levels of circulating TNF-α, IL1-β, IL-6, MIP1-α, and MCP-1. The influence of ZEBOV peptide on cytokine production was studied by stimulated human PBMC. At 40 μM concentration ZEBOV peptide suppressed anti-CD3/CD28-induced production of the Th1 cytokines IFN-γ (p<0.05; relative to control values) and IL-12p40 (p<0.05; relative to control values) (FIG. 10A). ZEBOV peptide also suppressed the production of the proliferative and differentiation factor IL-2 (p<0.05; relative to control values), and induced a dose-dependent reduction in TNF-α (p<0.05; relative to control values), IL-1β<0.01; relative to control values) and MCP-1 (p<0.01; relative to control values) (FIG. 10B). There was no effect on MIP1-α (FIG. 10B). ZEBOV peptide effects on Th2 cytokines were less consistent. ZEBOV peptide exposure resulted in an increase of IL-10 (p<0.01; relative to control values); a trend toward decrease was observed with IL-4; no pattern was observed with IL-6 (FIG. 10C). Cytokine data were fitted on a sigmoidal dose-response curve (variable slope) with R² values ranging from 0.8885 to 0.9748 (IL-2, IFN-Y, IL-12, TNF-α, and IL-1(3). The R² value for IL-10 was 0.7922. No effects were observed when human PBMC were exposed to REBOV peptide (FIG. 10A, B, C).

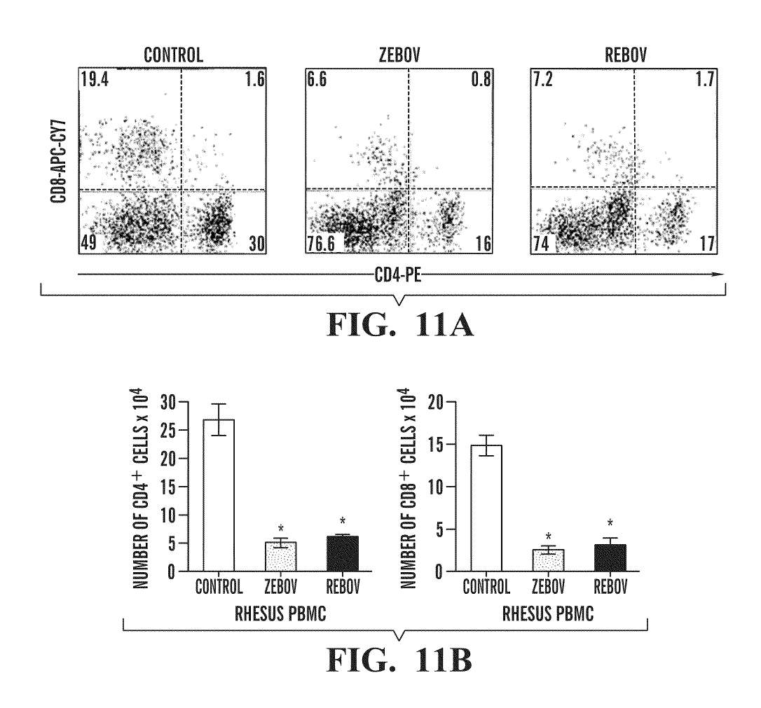

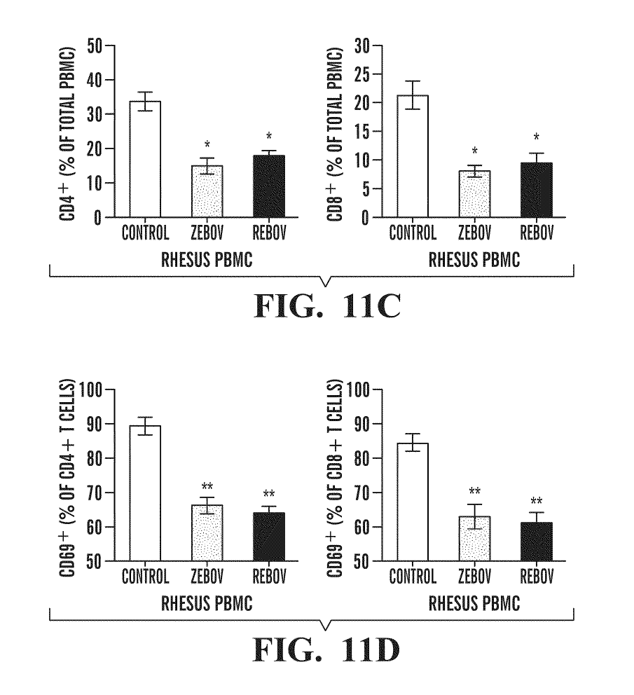

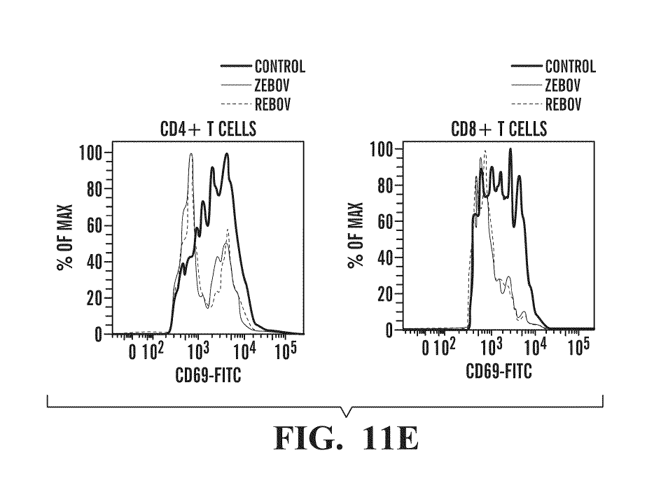

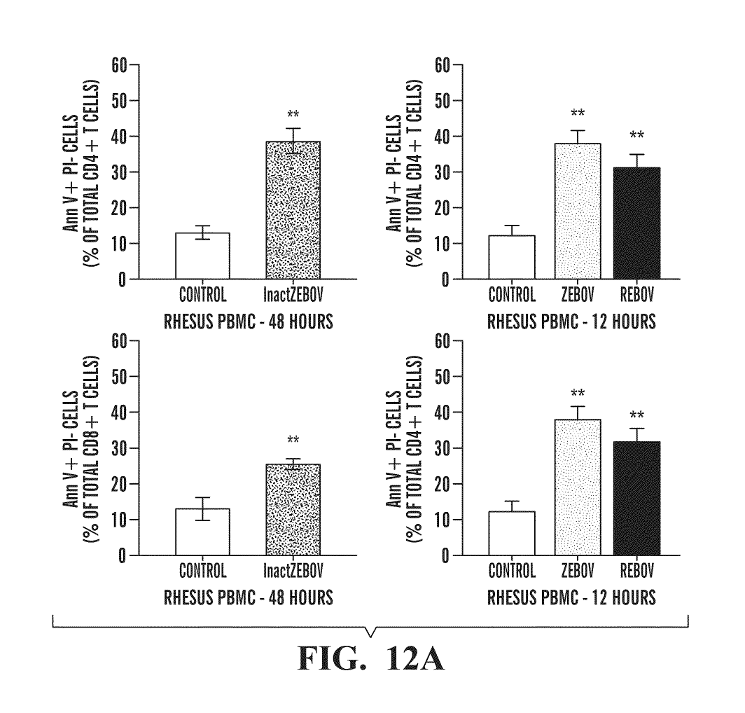

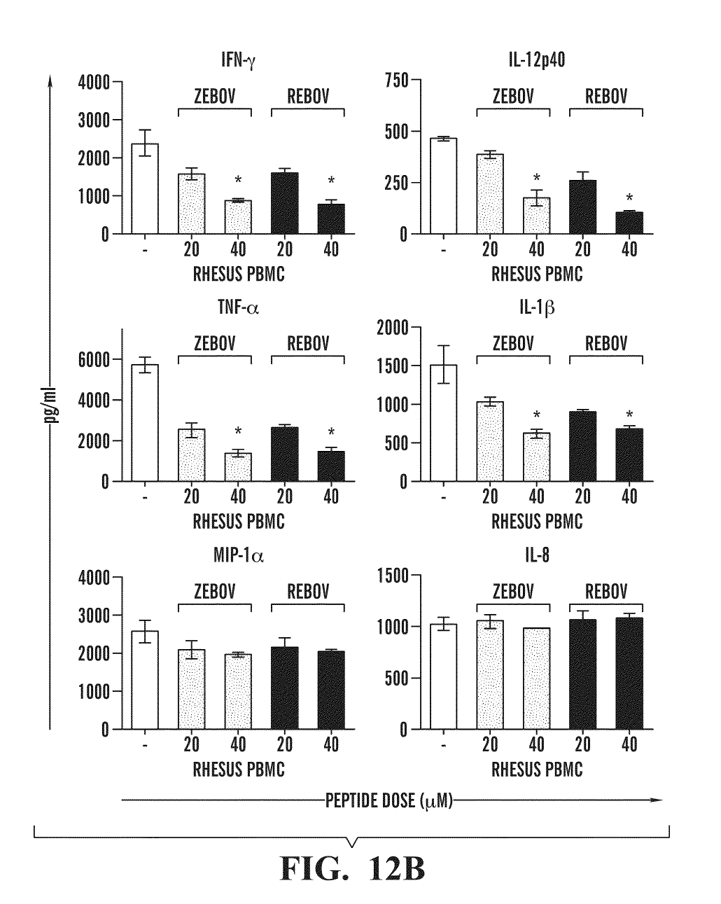

The observation that REBOV peptide had no effect on human PBMC in multiple assays was consistent with its lack of pathogenicity in humans. Given, however, that REBOV is pathogenic in monkeys, it was predicted that an immunosuppressive REBOV effect would be seen with monkey PBMC. To determine this, Rhesus macaque (Macaca mulatta) PBMC were incubated with REBOV peptide in the presence of anti-CD3 epsilon antibody. ZEBOV is pathogenic in monkeys as well as apes and humans; thus, ZEBOV peptide was used as a positive control. Significant depletion of CD4+ T cells and CD8+ T cells was observed with exposure to REBOV peptide or ZEBOV peptide (FIG. 11A, B). REBOV peptide exposure for 48 hours resulted in a 4.5-fold decrease in the number of CD4+ T cells and a 4.6-fold decrease in the number of CD8+ T cells (number of CD4+ T cells with REBOV peptide, n=5: 4.8±0.7×10⁴; number of CD8+ T cells without REBOV peptide, n=5: 26.6 t 2.7×10⁴; p<0.01; number of CD8+ T cells with REBOV peptide, n=5: 2.3±0.4×10⁴; number of CD8+ T cells without REBOV peptide, n=5: 14.7±1.2×10⁴; p<0.01; FIG. 11C). REBOV peptide exposure resulted in a decrease in the percentages of CD69+ cells in both CD4+ and CD8+ T cell populations (percentage of CD4+ rhesus T cells treated with REBOV peptide that are CD69+, n=5: 63.6±1.9%; without REBOV peptide, n=5: 89.1±2.6%; p<0.01; percentage of CD8+ rhesus T cells treated with REBOV peptide that are CD69+, n=5: 60.7±3.1%; without REBOV peptide, n=5: 84.3±2.7%; p<0.01; FIG. 11D). The mean fluorescent intensity of CD69 expression was also decreased on CD4+ and CD8+ T cells treated with REBOV peptide (FIG. 11E). REBOV treatment for 12 hours resulted in a 3.1-fold increase in apoptotic CD4+ cells and a 2.6-fold increase in apoptotic CD8+ cells (percentage of Annexin V+ PI− rhesus CD4+ treated with REBOV peptide, n=5: 46.0 f 2.1%; untreated cells, n=5: 14.0±3.0%; p<0.01; percentage of Annexin V+ P1− CD8+ treated with REBOV peptide, n=5: 31.8±3.3%; untreated cells, n=5: 12.3+3.0%; p<0.01; FIG. 12A). Decreased T cell activation and increased apoptosis were also observed with ZEBOV peptide and inactZEBOV (FIGS. 12C, D, E and FIG. 7A). Both ZEBOV peptide and REBOV peptide at a dose of 401M cause a significant decrease in Th 1 (IFN-γ and IL-12p40; p<0.05) and inflammatory cytokines (TNF-α and IL1-β; p<0.05) (FIG. 12B) compared with control values. Levels of IL-8 and MIP1-α did not alter with exposure to either peptide (FIG. 12B).

Examination of Immunosuppression, In Vitro, Using Human Peripheral Blood Mononuclear Cells (PBMCs).

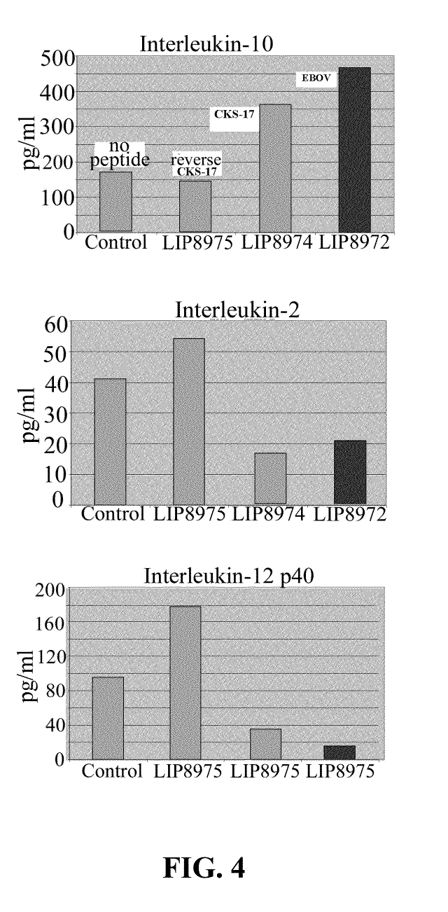

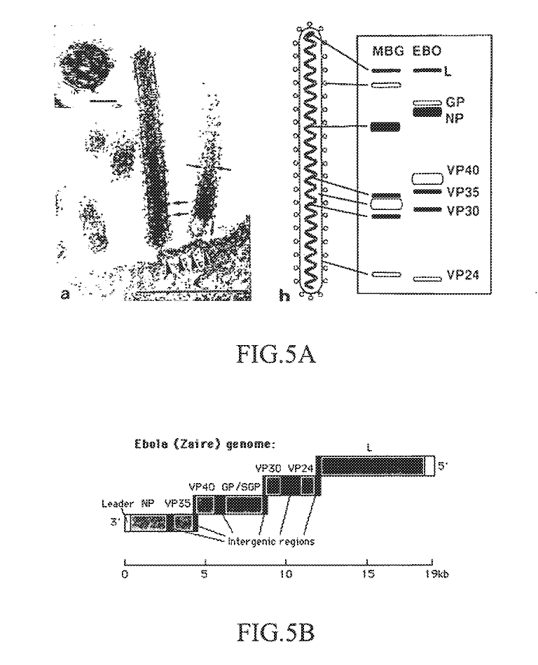

PBMCs from a healthy volunteer were obtained by density gradient centrifugation on Ficoll-hypaque (Pharmacia). PBMC were cultured at 10⁶/ml with stimulants (SEA, LPS, PHA, PMA+IONOMYCIN) with or without retroviral peptide CKS-17 (LIP8974), a negative control peptide with identical amino acid composition but reverse order (LIP8975) or Ebola Peptide with SEQ ID NO:1 (L1P8972) in RPMI1640 medium supplemented with 10% FBS and antibiotics at 37° C. for 5 and 12 hours. At the indicated time points, supernatant was collected from each well, aliquoted and tested for cytokine expression by Luminex using the beadlyte human multi-cytokine flex kit (Upstate Cell Signalling Solutions). The supernatant was also tested by Luminex for expression TNF-α, IFN-γ, IL-4, IL-6. FIG. 4 shows increased levels of IL-10 and decreased levels of IL-2 and IL-12 with exposure to Ebola or CKS-17 peptide. These findings are consistent with the peptides of the invention mediating immunosuppression. These results are examined with PBMC from other human volunteers.

To ascertain whether cytokine abnormalities are affected at the levels of mRNA, levels of the cognate transcripts will be quantitated in PBMC exposed to the relevant peptides. Real Time PCR assays will be used to quantitate mRNA encoding human IL-2, IL-10, IL-6, TNF-α, IL-γ, GADPH and β-actin.

To study signaling pathways and effectors of the immunosuppressive effect, the transcription and/or protein expression profiles of the PBMC exposed to filovirus peptides of the invention will be studied. Methods for such analyses are well known in the art.

Dimerized peptide may represent more accurately the structure shown in FIG. 3. The properties of the dimeric peptides, including immunosuppressive properties, will be investigated as described herein for a monomer peptide.

Longer polypeptides (e.g., 18, 19, 20, 21, 22, 23, 24, 25-mer), BSA-coupled polypeptides, pegylated peptides, or peptides modified by any suitable method will be synthesized and characterized for their immunosuppressive activity as described herein.

Identification of Agents which Modulate Immunosuppressive Function of Filoviral Peptides:

To identify possible therapeutic agents, including small molecules, or biological agents, which would modulate the immunosuppressive function of the peptide, PBMCs are isolated and grown as described herein. PBMCs from animals, or a healthy volunteer are obtained by density gradient centrifugation on Ficoll-hypaque (Pharmacia). PBMCs are cultured at 10⁶/ml with stimulants (SEA, LPS, PHA, PMA+IONOMYCIN), or in the presence of anti-CD28 antibody, anti-CD3 antibody, or a combination of anti-CD28/anti-CD3 antibodies, and are treated Ebola Peptide of SEQ ID NO:1 in RPMI1640 medium supplemented with 10% FBS and antibiotics at 37° C. for 5 and 12 hours. Potential, candidate therapeutic agents, for example a library of small compounds, will be added to the culture before or at different time points after the addition of the Ebola peptide of SEQ ID NO:1, or any of the other peptides of the invention. No therapeutic agent will be added to a control culture in which the PBMCs are treated with the Ebola peptide of SEQ ID NO:1, or any of the other peptides of the invention. To ascertain the effect of the potentially therapeutic gents, the ability of PBMCs to proliferate will be measured by the lymphoproliferation assay, BrdU labeling and cell cycle analysis, assays for apoptosis, assays to measure cytokine expression, or any other suitable assay, including but not limited to the methods and assays used herein to characterize the effect of filoviral peptides on PBMCs. A therapeutic agent that decreases the immunosuppressive activity of the Ebola peptide will improve the proliferative ability of PBMCs treated with the Ebola peptide. This assay will also identify an agent that increases the immunosuppressive activity of the Ebola peptide. Addition of such agent will result in a further decrease in the proliferative ability of PBMCs treated with the Ebola peptide of SEQ ID NO:1. The effect of a potentially therapeutic compound can also be determined by measuring the levels of cytokines, such as IL-10, IL-2, and IL-12. A therapeutic agent that decreases the immunosuppressive activity of the Ebola peptide will increase the levels of cytokines produced by PBMCs treated with the Ebola peptide.

TABLE 1
Isolated therapeutic peptides

| | | | | | | | |
|---|---|---|---|---|---|---|---|
| ENV_AVIRE | 441 | 519 | SEQ ID NO: 129 | LQNRRGLDLLTAEQGGIC | P03399 | Envelope | Avian |
| | | | | | | glycoprotein | reticuloendotheliosis |
| | | | | | | precursor | virus. |
| ENV_AVISN | 447 | 525 | SEQ ID NO: 130 | LQNRRGLDLLTAEQGGIC | P31796 | Envelope | Avian spleen |
| | | | | | | glycoprotein | necrosis virus. |
| | | | | | | precursor | |
| ENV_AVISU | 1 | 114 | SEQ ID NO: 131 | LQNRAAIDFLLLAHGHGC | P03398 | Envelope | Avian sarcoma |
| | | | | | | glycoprotein | virus (strain UR2). |
| ENV_BAEVM | 415 | 503 | SEQ ID NO: 132 | LQNRRGLDLLTAEQGGIC | P10269 | Envelope | Baboon |
| | | | | | | glycoprotein | endogenous virus |
| | | | | | | precursor | (strain M7). |
| ENV_FENV1 | 524 | 607 | SEQ ID NO: 133 | LQNRRGLDLLFLQEGGLC | P31791 | Envelope | Feline endogenous |
| | | | | | | glycoprotein | virus ECE1. |
| | | | | | | precursor | |
| ENV_FLVC6 | 518 | 601 | SEQ ID NO: 134 | LQNRRGLDILFLQEGGLC | P21443 | Envelope | Feline leukemia |
| | | | | | | glycoprotein | provirus (isolate |
| | | | | | | precursor | CFE-6). |
| ENV_FLVGL | 499 | 582 | SEQ ID NO: 135 | LQNRRGLDILFLQEGGLC | P08359 | Envelope | Feline leukemia |
| | | | | | | glycoprotein | virus (strain |
| | | | | | | precursor | A/Glasgow-1). |
| ENV_FLVSA | 496 | 579 | SEQ ID NO: 136 | LQNRRGLDILFLQEGGLC | P06752 | Envelope | Feline leukemia |
| | | | | | | glycoprotein | virus (strain |
| | | | | | | precursor | C/Sarma). |
| ENV_FSVGA | 519 | 602 | SEQ ID NO: 137 | LQNRRGLDILFLQEGGLC | P03391 | Envelope | Feline sarcoma |
| | | | | | | glycoprotein | virus (strain |
| | | | | | | precursor | Gardner-Arnstein) |
| | | | | | | | (Ga-FeSV) |
| | | | | | | | (Gardner |
| ENV_FSVSM | 502 | 585 | SEQ ID NO: 138 | LQNRRGLDILFLQGGGLC | P21445 | Envelope | Feline sarcoma |
| | | | | | | glycoprotein | virus (strain SM) |
| | | | | | | precursor | (Sm-FeSV). |
| ENV_GALV | 547 | 622 | SEQ ID NO: 139 | LQNRRGLDLLFLKEGGLC | P21415 | Envelope | Gibbon ape |
| | | | | | | glycoprotein | leukemia virus |
| | | | | | | precursor | (GALV). |
| ENV_HTL1A | 346 | 408 | SEQ ID NO: 140 | AQNRRGLDLLFWEQGGLC | P03381 | Envelope | Human T-cell |
| | | | | | | glycoprotein gp62 | leukemia virus 1 |
| | | | | | | precursor | (strain ATK) |
| | | | | | | | (HTLV-1). |
| ENV_HTL1C | 346 | 439 | SEQ ID NO: 141 | AQNRRGLDLLFWEQGGLC | P14075 | Envelope | Human T-cell |
| | | | | | | glycoprotein gp62 | leukemia virus 1 |
| | | | | | | precursor | (isolate Caribbea) |
| | | | | | | | (HTLV-1). |
| ENV_HTL1F | 346 | 439 | SEQ ID NO: 142 | AQNRRGLDLLFWEQGGLC | Q03817 | Envelope | Human T-cell |
| | | | | | | glycoprotein gp62 | leukemia virus 1 |
| | | | | | | precursor | (isolate Africa) |
| | | | | | | | (HTLV-1). |
| ENV_HTL1M | 346 | 439 | SEQ ID NO: 143 | AQNRRGLDLLFWEQGGLC | P23064 | Envelope | Human T-cell |
| | | | | | | glycoprotein gp62 | leukemia virus 1 |
| | | | | | | precursor | (isolate MT-2) |
| | | | | | | | (HTLV-1). |
| ENV_HTL1N | 346 | 439 | SEQ ID NO: 144 | AQNRRGLDLLFWEQGGLC | Q03816 | Envelope | Human T-cell |
| | | | | | | glycoprotein gp62 | leukemia virus 1 |
| | | | | | | precursor | (isolate North |
| | | | | | | | America) (HTLV-1). |
| ENV_HTLV2 | 373 | 435 | SEQ ID NO: 145 | -QNRRGLDLLFWEQGGLC | P03383 | Envelope | Human T-cell |
| | | | | | | glycoprotein gp63 | leukemia virus 2 |
| | | | | | | precursor | (HTLV-2). |
| Q9TTC0_PHACI | 518 | 596 | SEQ ID NO: 146 | LQNRRGLDLLFLKEGGLC | Q9TTC0 | Envelope | Koala retrovirus |
| | | | | | | glycoprotein | (KoRV). |
| | | | | | | precursor | |
| ENV_MCFF | 494 | 577 | SEQ ID NO: 147 | LQNRRGLDLLFLKEGGLC | P15073 | Envelope | Mink cell focus |
| | | | | | | glycoprotein | forming murine |
| | | | | | | precursor | leukemia virus. |
| ENV_MCFF3 | 495 | 578 | SEQ ID NO: 148 | LQNRRGLDLLFLKEGGLC | P03388 | Envelope | Mink cell focus- |
| | | | | | | glycoprotein | forming murine |
| | | | | | | precursor | leukemia virus |
| | | | | | | | (isolate CI-3). |
| ENV_MLVAV | 524 | 607 | SEQ ID NO: 149 | LQNRRGLDLLFLKEGGLC | P03386 | Envelope | AKV murine |
| | | | | | | glycoprotein | leukemia virus (AKR |
| | | | | | | precursor | (endogenous) |
| | | | | | | | murine leukemia |
| | | | | | | | virus |
| ENV_MLVCB | 519 | 602 | SEQ ID NO: 150 | LQNRRGLDLLFLKEGGLC | P08360 | Envelope | CasBrE murine |
| | | | | | | glycoprotein | leukemia virus. |
| | | | | | | precursor | |
| ENV_MLVF5 | 533 | 616 | SEQ ID NO: 151 | LQNRRGLDLLFLKEGGLC | P03390 | Envelope | Friend murine |
| | | | | | | glycoprotein | leukemia virus |
| | | | | | | precursor | (isolate 57) |
| | | | | | | | (FrMLV). |
| ENV_MLVFF | 533 | 616 | SEQ ID NO: 152 | LQNRRGLDLLFLKEGGLC | P26804 | Envelope | Friend murine |
| | | | | | | glycoprotein | leukemia virus |
| | | | | | | precursor | (isolate FB29) |
| | | | | | | | (FrMLV). |
| ENV_MLVFP | 533 | 616 | SEQ ID NO: 153 | LQNRRGLDLLFLKEGGLC | P26803 | Envelope | Friend murine |
| | | | | | | glycoprotein | leukemia virus |
| | | | | | | precursor | (isolate PVC-211) |
| | | | | | | | (FrMLV). |
| ENV_MLVHO | 510 | 603 | SEQ ID NO: 154 | LQNRRGLDLLFLEKGGLC | P21436 | Envelope | Hortulanus murine |
| | | | | | | glycoprotein | leukemia virus |
| | | | | | | precursor | (HoMuLV) (Mus |
| | | | | | | | hortulanus virus). |
| ENV_MLVMO | 523 | 606 | SEQ ID NO: 155 | LQNRRGLDLLFLKEGGLC | P03385 | Envelope | Moloney murine |
| | | | | | | glycoprotein | leukemia virus |
| | | | | | | precursor | (MoMLV). |
| ENV_MLVRD | 518 | 601 | SEQ ID NO: 156 | LQNRRGLDLLFLKEGGLC | P11268 | Envelope | Radiation murine |
| | | | | | | glycoprotein | leukemia virus. |
| | | | | | | precursor | |
| ENV_MLVRK | 518 | 601 | SEQ ID NO: 157 | LQNRRGLDLLFLKEGGLC | P31794 | Envelope | Radiation murine |
| | | | | | | glycoprotein | leukemia virus |
| | | | | | | precursor | (strain Kaplan). |
| ENV_MPMV | 440 | 507 | SEQ ID NO: 158 | LQNRRGLDLLTAEQGGIC | P07575 | Envelope | Mason-Pfizer |
| | | | | | | glycoprotein | monkey virus |
| | | | | | | precursor | (MPMV) (Simian |
| | | | | | | | Mason-Pfizer virus). |
| ENV_RMCFV | 497 | 580 | SEQ ID NO: 159 | LQNRRGLDLLFLKEGGLC | P06445 | Envelope | Rauscher mink cell |
| | | | | | | glycoprotein | focus-inducing |
| | | | | | | precursor | virus. |
| ENV_RSVP | 436 | 549 | SEQ ID NO: 160 | LQNRAAIDFLLLAHGHGC | P03396 | Envelope | Rous sarcoma |
| | | | | | | glycoprotein gp95 | virus (strain Prague |
| | | | | | | precursor | C) (RSV-PrC). |
| ENV_SMRVH | 413 | 499 | SEQ ID NO: 161 | LQNRRGLDLLTAEQGGIC | P21412 | Envelope | Squirrel monkey |
| | | | | | | glycoprotein | retrovirus (SMRV-H) |
| | | | | | | precursor | (SMRV-HLB). |
| ENV_SRV1 | 423 | 523 | SEQ ID NO: 162 | LQNRRGLDLLTAEQGGIC | P04027 | Envelope | Simian retrovirus |
| | | | | | | glycoprotein | SRV-1. |
| | | | | | | precursor | |
| ENV_SRV2 | 428 | 497 | SEQ ID NO: 163 | LQNRRGLDLLTAEQGGIC | P51515 | Envelope | Simian retrovirus |
| | | | | | | glycoprotein | SRV-2. |
| | | | | | | precursor | |
| ENV_SRV2R | 428 | 497 | SEQ ID NO: 164 | LQNRRGLDLFTAEQGGIC | P51520 | Envelope | Simian retrovirus |
| | | | | | | glycoprotein | SRV-2 (isolate 2R- |
| | | | | | | precursor | 18B1). |
| ENV2_MOUSE | 532 | 615 | SEQ ID NO: 165 | LQNRRGLDLLFLKEGGLC | P11370 | Retrovirus | Mus musculus |
| | | | | | | related | (Mouse). |
| | | | | | | Env polyprotein | |
| | | | | | | from Fv4 locus | |
| Q9UQF0_HUMAN | 368 | 447 | SEQ ID NO: 166 | LQNRRALDLLTAERGGTC | Q9UQF0 | HERV-W_7q21.2 | Homo sapiens |
| | | | | | | (Syncytin-1) | (Human). |
| O09243_VVVVV | 373 | 435 | SEQ ID NO: 167 | -QNRRGLDLLFWEQGGLC | O09243 | Envelope | Simian T- |
| | | | | | | protein | lymphotropic virus |
| | | | | | | | 2. |
| O12374_VVVVV | 533 | 616 | SEQ ID NO: 168 | LQNRRGLDLLFLKEGGLC | O12374 | Polyprotein | Murine leukemia |
| | | | | | | | virus. |
| O36258_VVVVV | 346 | 439 | SEQ ID NO: 169 | AQNRRGLDLLFWEQGGLC | O36258 | Envelope | Human T- |
| | | | | | | glycoprotein | lymphotropic virus |
| | | | | | | | 1. |
| O36428_VVVVV | 546 | 644 | SEQ ID NO: 170 | LINRHAIDFLLTRWGGTC | O36428 | Glycoprotein | Lake Victoria |
| | | | | | | precursor | marburgvirus. |
| O36429_VVVVV | 546 | 644 | SEQ ID NO: 171 | LINRHAIDFLLTRWGGTC | O36429 | Glycoprotein | Lake Victoria |
| | | | | | | precursor | marburgvirus. |
| O39737_MLVFR | 533 | 616 | SEQ ID NO: 172 | LQNRRGLDLLFLKEGGLC | O39737 | Envelope | Friend murine |
| | | | | | | protein | leukemia virus |
| | | | | | | | (FrMLV). |
| O41172_VVVVV | 518 | 572 | SEQ ID NO: 173 | LQNRRGLDLLFLKEGGLC | O41172 | Env protein | Porcine |
| | | | | | | | endogenous |
| | | | | | | | retrovirus. |
| O41173_VVVVV | 515 | 569 | SEQ ID NO: 174 | LQNRRGLDLLFLREGGLC | O41173 | Env protein | Porcine |
| | | | | | | | endogenous |
| | | | | | | | retrovirus. |
| O41251_MLVRA | 533 | 616 | SEQ ID NO: 175 | LQNRRGLDLLFLKEGGLC | O41251 | Env | Rauscher murine |
| | | | | | | polyprotein | leukemia virus (R- |
| | | | | | | | MuLV). |
| O41441_FLV | 503 | 586 | SEQ ID NO: 176 | LQNRRGLDILFLQEGGLC | O41441 | Envelope | Feline leukemia |
| | | | | | | polyprotein | virus. |
| O41897_VVVVV | 346 | 439 | SEQ ID NO: 177 | AQNRRGLDLLFWEQGGLC | O41897 | Envelope | Simian T- |
| | | | | | | glycoprotein | lymphotropic virus |
| | | | | | | | 1. |
| O62705_PIG | 497 | 549 | SEQ ID NO: 178 | LQNRRGLDLLFLKEGGLC | O62705 | Env protein | Sus scrofa (Pig). |
| O62707_PIG | 497 | 549 | SEQ ID NO: 179 | LQNRRGLDLLFLKEGGLC | O62707 | Env protein | Sus scrofa (Pig). |
| O70644_VVVVV | 373 | 435 | SEQ ID NO: 180 | -QNRRGLDLLFWEQGGLC | O70644 | Env protein | Simian T- |
| | | | | | | | lymphotropic virus |
| | | | | | | | 2. |
| 070653_GALV | 529 | 607 | SEQ ID NO: 181 | LQNRRGLDLLFLKEGGLC | O70653 | Envelope | Gibbon ape |
| | | | | | | protein | leukemia virus |
| | | | | | | | (GALV). |
| O70942_VVVVV | 514 | 566 | SEQ ID NO: 182 | LQNRRGLDLLFLREGGLC | O70942 | Envelope | Porcine |
| | | | | | | protein | endogenous |
| | | | | | | | retrovirus. |
| O73456_VVVVV | 373 | 435 | SEQ ID NO: 183 | -QNRRGLDLLFWEQGGLC | O73456 | Env protein | Human T-cell |
| | | | | | | | lymphotropic virus |
| | | | | | | | type 2b. |
| O73506_VVVVV | 515 | 569 | SEQ ID NO: 184 | LQNRRGLDLLFLREGGLC | O73506 | Env protein | Porcine |
| | | | | | | | endogenous |
| | | | | | | | retrovirus. |
| O89812_FLV | 499 | 582 | SEQ ID NO: 185 | LQNRRGLDILFLQEGGLC | O89812 | Env gene | Feline leukemia |
| | | | | | | polyprotein | virus. |
| O89816_VVVVV | 505 | 614 | SEQ ID NO: 186 | LQNRRGLDLLFLKEGGLC | O89816 | Envelope | Mus dunni |
| | | | | | | glycoprotein | endogenous virus. |
| O92789_FRSFV | 533 | 609 | SEQ ID NO: 187 | LQNRRGLDLLFLKEGGLC | O92789 | Envelope protein | Friend spleen |
| | | | | | | | focus-forming virus |
| | | | | | | | (FSFFV). |
| O92955_VVVVV | 439 | 552 | SEQ ID NO: 188 | LQNRAAIDFLLLAHGHGC | O92955 | Envelope | Rous sarcoma |
| | | | | | | glycopolyprotein | virus (strain |
| | | | | | | | Schmidt-Ruppin B) |
| | | | | | | | (RSV-SR8). |
| P70356_MOUSE | 524 | 607 | SEQ ID NO: 189 | LQNRRGLDLLFLKEGGLC | P70356 | Envelope protein | Mus musculus |
| | | | | | | | (Mouse). |
| P88820_VVVVV | 346 | 439 | SEQ ID NO: 190 | AQNRRGLDLLFWEQGGLC | P88820 | Envelope | Human T- |
| | | | | | | glycoprotein | lymphotropic virus |
| | | | | | | | 1. |
| P88821_VVVVV | 346 | 439 | SEQ ID NO: 191 | AQNRRGLDLLFWEQGGLC | P88821 | Envelope | Human T- |
| | | | | | | glycoprotein | lymphotropic virus |
| | | | | | | | 1. |
| P90198_VVVVV | 346 | 439 | SEQ ID NO: 192 | AQNRRGLDLLFWEQGGLC | P90198 | Envelope | Human T- |
| | | | | | | glycoprotein | lymphotropic virus |
| | | | | | | | 1. |
| P90199_VVVVV | 346 | 439 | SEQ ID NO: 193 | AQNRRGLDLLFWEQGGLC | P90199 | Envelope | Human T- |
| | | | | | | glycoprotein | lymphotropic virus |
| | | | | | | | 1. |
| P90200_VVVVV | 346 | 439 | SEQ ID NO: 194 | AQNRRGLDLLFWEQGGLC | P90200 | Envelope | Human T- |
| | | | | | | glycoprotein | lymphotropic virus |
| | | | | | | | 1. |
| P90201_VVVVV | 346 | 439 | SEQ ID NO: 195 | AQNRRGLDLLFWEQGGLC | P90201 | Envelope | Human T- |
| | | | | | | glycoprotein | lymphotropic virus |
| | | | | | | | 1. |
| P90202_VVVVV | 346 | 439 | SEQ ID NO: 196 | AQNRRGLDLLFWEQGGLC | P90202 | Envelope | Human T- |
| | | | | | | glycoprotein | lymphotropic virus |
| | | | | | | | 1. |
| P97406_MOUSE | 461 | 554 | SEQ ID NO: 197 | LRNQREQDFQSLQQDGLC | P97406 | Viral envelope | Mus musculus |
| | | | | | | like | (Mouse). |
| | | | | | | protein (Proviral | |
| | | | | | | envelope protein) | |
| Q01280_VVVVV | 541 | 624 | SEQ ID NO: 198 | LQNRRGLDLLFLKEGGLC | Q01280 | Env protein | Retroviridae. |
| Q01281_VVVVV | 541 | 624 | SEQ ID NO: 199 | LQNRRGLDLLFLKEGGLC | Q01281 | Env protein | Retroviridae. |
| Q03803_ALV | 432 | 545 | SEQ ID NO: 200 | LQNRAAIDFLLLAHGHGC | P03397 | Env polyprotein | Avian leukosis virus |
| | | | | | | | RSA (RSV-SRA) |
| | | | | | | | (Rous sarcoma |
| | | | | | | | virus (strain |
| | | | | | | | Schmidt-Ruppin A)) |
| Q03813_VVVVV | 346 | 439 | SEQ ID NO: 201 | AQNRRGLDLLFWEQGGLC | Q03813 | Envelope protein | Human T- |
| | | | | | | | Iymphotropic virus |
| | | | | | | | 1. |
| Q03819_VVVVV | 428 | 541 | SEQ ID NO: 202 | LQNRAAIDFLLLAHGHGC | Q03819 | Gp37 (Gp85) | Rous sarcoma |
| | | | | | | | virus. |
| Q03822_VVVVV | 346 | 439 | SEQ ID NO: 203 | AQNRRGLDLLFWEQGGLC | Q03822 | Simian T-cell | Simian T- |
| | | | | | | leukemia virus, | lymphotropic virus |
| | | | | | | pol-env-pX-3′ | 1. |
| | | | | | | LTR region | |
| | | | | | | (Envelope protein) | |
| Q03875_VVVVV | 472 | 580 | SEQ ID NO: 204 | LQNRRGLDLLFLKEGGLC | Q03875 | Gp70 protein | Murine leukemia |
| | | | | | | | virus. |
| Q04586_MOUSE | 524 | 607 | SEQ ID NO: 205 | LQNRRGLDLLFLKEGGLC | Q04586 | Env polyprotein | Mus musculus |
| | | | | | | | (Mouse). |
| Q07453_VVVVV | 410 | 523 | SEQ ID NO: 206 | LQNRAAIDFLLLAHGHGC | Q07453 | Gp85 | Rous sarcoma |
| | | | | | | (5246 . . . 6268) | virus. |
| | | | | | | (Gp37 | |
| | | | | | | (6269 . . . 6865)) | |
| Q08829_HTLV2 | 373 | 435 | SEQ ID NO: 207 | -QNRRGLDLLFWEQGGLC | Q08829 | Env protein | Human T-cell |
| | | | | | | | leukemia virus 2 |
| | | | | | | | (HTLV-2). |
| Q14264_HUMAN | 475 | 562 | SEQ ID NO: 208 | YQNRLALDYLLAQEEGVC | Q14264 | HERV-R_7q21.2 | Homo sapiens |
| | | | | | | provirus | (Human). |
| | | | | | | ancestral Env | |
| | | | | | | polyprotein | |
| | | | | | | precursor | |
| Q60589_MOUSE | 524 | 607 | SEQ ID NO: 209 | LQNRRGLDLLFLKEGGLC | Q60589 | Envelope | Mus musculus |
| | | | | | | glycoprotein | (Mouse). |
| Q61876_MOUSE | 418 | 526 | SEQ ID NO: 210 | LQNRRGLDLLFLKEGGLC | Q61876 | Endogenous murine | Mus musculus |
| | | | | | | leukemia virus | (Mouse). |
| | | | | | | polytropic provirus | |
| | | | | | | DNA, complete cds | |
| Q61877_MOUSE | 460 | 568 | SEQ ID NO: 211 | LQNRRGLDLLFLKEGGLC | Q61877 | Envelope protein | Mus musculus |
| | | | | | | | (Mouse). |
| Q61919_MOUSE | 524 | 607 | SEQ ID NO: 212 | LQNRRGLDLLFLKEGGLC | Q61919 | Envelope protein | Mus musculus |
| | | | | | | | (Mouse). |
| Q64984_RSVP | 434 | 547 | SEQ ID NO: 213 | LQNRAAIDFLLLAHGHGC | Q64984 | Env-Pr95 | Rous sarcoma |
| | | | | | | polyprotein | virus (strain Prague |
| | | | | | | | C) (RSV-PrC). |
| Q64997_ALV | 403 | 515 | SEQ ID NO: 214 | LQNRAAIDFLLLAQGHGC | Q64997 | Envelope protein | Avian leukosis virus |
| | | | | | | subgroup J | HPRS103. |
| Q65731_VVVVV | 346 | 439 | SEQ ID NO: 215 | AQNRRGLDLLFWEQGGLC | Q65731 | Envelope protein | Baboon T-cell |
| | | | | | | | leukemia virus. |
| Q66818_VVVVV | 554 | 648 | SEQ ID NO: 216 | ILNRKAIDFLLQRWGGTC | Q05320 | Envelope | Zaire ebolavirus |
| | | | | | | glycoprotein | (strain Mayinga-76) |
| | | | | | | | (ZEBOV) (Zaire |
| | | | | | | | Ebola virus) |
| Q66917_FLV | 499 | 582 | SEQ ID NO: 217 | LQNRRGLDILFLQEGGLC | Q66917 | Glycoprotein gp70 | Feline leukemia |
| | | | | | | precursor | virus. |
| Q67456_MLVFR | 533 | 616 | SEQ ID NO: 218 | LQNRRGLDLLFLKEGGLC | Q67456 | Viral envelope | Friend murine |
| | | | | | | protein | leukemia virus |
| | | | | | | precursor | (FrMLV). |
| Q67649_GALV | 525 | 608 | SEQ ID NO: 219 | LQNRRGLDLLFLKEGGLC | Q67649 | Envelope protein | Gibbon ape |
| | | | | | | | leukemia virus |
| | | | | | | | (GALV). |
| Q7ZFQ2_ALV | 405 | 517 | SEQ ID NO: 220 | LONRAAIDFLLLAQGHGC | Q7ZFQ2 | Envelope protein | Avian leukosis virus |
| | | | | | | | (ALV). |
| Q7ZGR3_VVVVV | 533 | 616 | SEQ ID NO: 221 | LQNRRGLDLLFLKEGGLC | Q7ZGR3 | Envelope protein | Murine leukemia |
| | | | | | | | virus. |
| Q7ZGS2_VVVVV | 505 | 609 | SEQ ID NO: 222 | YQNRLALDYLLAAEGGVC | Q7ZGS2 | Env protein | Human |
| | | | | | | | endogenous |
| | | | | | | | retrovirus HCML- |
| | | | | | | | ARV. |
| Q7ZJT7_VVVVV | 493 | 595 | SEQ ID NO: 223 | LQNRRGLDLLFLKEGGLC | Q7ZJT7 | Envelope | Amphotropic Murine |
| | | | | | | polyprotein | leukemia virus. |
| | | | | | | precursor | |
| Q7ZL00_VVVVV | 473 | 555 | SEQ ID NO: 224 | LQNRRGLDMLFLREGGLC | Q7ZL00 | Envelope | Recombinant M- |
| | | | | | | glycoprotein | MuLV/RaLV |
| | | | | | | | retrovirus. |
| Q7ZL02_VVVVV | 473 | 555 | SEQ ID NO: 225 | LQNRRGLDMLFLREGGLC | Q7ZL02 | Envelope | Recombinant M- |
| | | | | | | glycoprotein | MuLV/RaLV |
| | | | | | | | retrovirus. |
| Q7ZZV5_CHICK | 403 | 515 | SEQ ID NO: 226 | LQNRAAIDFLLLAQGHGC | Q7ZZV5 | Envelope protein | Gallus gallus |
| | | | | | | | (Chicken). |
| Q80792_VVVVV | 346 | 439 | SEQ ID NO: 227 | AQNRRGLDLLFWEQGGLC | Q80792 | Envelope protein | Human T- |
| | | | | | | | lymphotropic virus |
| | | | | | | | 1. |
| Q80810_VVVVV | 345 | 439 | SEQ ID NO: 228 | AQNRRGLDLLFWEQGGLC | Q80810 | Envelope | Human T- |
| | | | | | | glycoprotein gp46 | lymphotropic virus |
| | | | | | | precursor | 1. |
| Q82234_VVVVV | 346 | 439 | SEQ ID NO: 229 | AQNRRGLDLLFWEQGGLC | Q82234 | Env protein | Human T- |
| | | | | | | | lymphotropic virus |
| | | | | | | | 1. |
| Q82325_VVVVV | 346 | 439 | SEQ ID NO: 230 | AQNRRGLDLLFWEQGGLC | Q82325 | Envelope | Human T- |
| | | | | | | glycoprotein | lymphotropic virus |
| | | | | | | | 1. |
| Q82339_HTLV2 | 373 | 435 | SEQ ID NO: 231 | -QNRRGLDLLFWEQGGLC | Q82339 | Orf protein | Human T-cell |
| | | | | | | | leukemia virus 2 |
| | | | | | | | (HTLV |
| Q82345_HTLV2 | 373 | 435 | SEQ ID NO: 232 | -QNRRGLDLLFWEQGGLC | Q82345 | Env protein | Human T-cell |
| | | | | | | | leukemia virus 2 |
| | | | | | | | (HTLV-2). |
| Q83129_VVVVV | 433 | 546 | SEQ ID NO: 233 | LQNRAAIDFLLLAHGHGC | Q83129 | Env protein | Avian |
| | | | | | | | myeloblastosis- |
| | | | | | | | associated virus |
| | | | | | | | 1/2. |
| Q83132_VVVVV | 437 | 550 | SEQ ID NO: 234 | LQNRAAIDFLLLAHGHGC | Q83132 | Env protein | Avian |
| | | | | | | | myeloblastosis- |
| | | | | | | | associated virus |
| | | | | | | | type 1. |
| Q83134_VVVVV | 440 | 553 | SEQ ID NO: 235 | LQNRAAIDFLLLAHGHGC | Q83134 | Env protein | Avian |
| | | | | | | | myeloblastosis- |
| | | | | | | | associated virus |
| | | | | | | | type 2. |
| Q83363_VVVVV | 494 | 577 | SEQ ID NO: 236 | LQNRRGLDLLFLKEGGLC | Q83363 | Env polyprotein | Murine leukemia |
| | | | | | | | virus. |
| Q83364_MLVMO | 491 | 593 | SEQ ID NO: 237 | LQNRRGLDLLFLKEGGLC | Q83364 | GPr80 envelope | Murine leukemia |
| | | | | | | polyprotein | virus. |
| Q83365_MLVMO | 475 | 577 | SEQ ID NO: 238 | LQNRRGLDLLFLKEGGLC | Q83365 | GPr80 envelope | Murine leukemia |
| | | | | | | polyprotein | virus. |
| Q83375_VVVVV | 484 | 586 | SEQ ID NO: 239 | LQNRRGLDLLFLKEGGLC | Q83375 | 10A1 Murine | Murine leukemia |
| | | | | | | leukemia | virus. |
| | | | | | | virus envelope | |
| Q83380_VVVVV | 473 | 555 | SEQ ID NO: 240 | LQNRRGLDMLFLREGGLC | Q83380 | Envelope protein | Rat leukemia virus. |
| Q83382_VVVVV | 524 | 607 | SEQ ID NO: 241 | LQNRRGLDLLFLKEGGLC | Q83382 | Envelope | Murine leukemia |
| | | | | | | glycoprotein | virus. |
| Q83399_VVVVV | 542 | 625 | SEQ ID NO: 242 | LQNRRGLDLLFLKEGGLC | Q83399 | Envelope | Murine leukemia |
| | | | | | | glycoprotein | virus. |
| Q85091_VVVVV | 379 | 441 | SEQ ID NO: 243 | -QNRRGLDLLFWEQGGLC | Q85091 | Env protein | Simian T- |
| | | | | | | | lymphotropic virus |
| | | | | | | | 3. |
| Q85506_VVVVV | 494 | 577 | SEQ ID NO: 244 | LQNRRGLDLLFLKEGGLC | Q85506 | Env polyprotein | Murine leukemia |
| | | | | | | | virus. |
| Q85510_VVVVV | 472 | 580 | SEQ ID NO: 245 | LQNRRGLDLLFLKEGGLC | Q85510 | Envelope | Xenotropic murine |
| | | | | | | polyprotein | leukemia virus. |
| Q85518_FLV | 499 | 582 | SEQ ID NO: 246 | LQNRRGLDILFLQEGGLC | Q85518 | subgroup A (FeLV- | Feline leukemia |
| | | | | | | 3281-A), envelope | virus. |
| | | | | | | and LTR regions. | |
| | | | | | | precursor | |
| Q85522_FLV | 499 | 582 | SEQ ID NO: 247 | LQNRRGLDILFLQEGGLC | Q85522 | Env gene | Feline leukemia |
| | | | | | | polyprotein | virus. |
| | | | | | | precursor | |
| Q85525_FLV | 499 | 582 | SEQ ID NO: 248 | LQNRRGLDILFLQEGGLC | Q85525 | Envelope | Feline leukemia |
| | | | | | | polyprotein | virus. |
| | | | | | | precursor | |
| Q85630_FRMCV | 494 | 577 | SEQ ID NO: 249 | LQNRRGLDLLFLKEGGLC | Q85630 | Env protein | Friend mink cell |
| | | | | | | precursor | focus-inducing |
| | | | | | | | virus. |
| Q85735_VVVVV | 472 | 580 | SEQ ID NO: 250 | LQNRRGLDLLFLKEGGLC | Q85735 | Env protein | Murine type C |
| | | | | | | precursor | retrovirus. |
| Q86687_HTLV2 | 376 | 406 | SEQ ID NO: 251 | -QNRRGLDLLFWEQGGLC | Q86687 | Envelope | Human T-cell |
| | | | | | | glycoprotein | leukemia virus 2 |
| | | | | | | | (HTLV-2). |
| Q86UH7_HUMAN | 505 | 609 | SEQ ID NO: 252 | YQNRLALDYLLAAEGGVC | Q86UH7 | Envelope | Homo sapiens |
| | | | | | | glycoprotein | (Human). |
| Q89683_VVVVV | 493 | 595 | SEQ ID NO: 253 | LQNRRGLDLLFLKEGGLC | Q89683 | GPr80 envelope | Murine leukemia |
| | | | | | | polyprotein (4070A | virus. |
| | | | | | | Amphotropic Murine | |
| | | | | | | leukemia virus | |
| | | | | | | envelope) | |
| Q8AGK3_VVVVV | 378 | 440 | SEQ ID NO: 254 | -QNRRGLDLLFWEQGGLC | Q8AGK3 | Envelope protein | Simian T- |
| | | | | | | | lymphotropic virus |
| | | | | | | | 3. |
| Q8AGX8_VVVVV | 467 | 550 | SEQ ID NO: 255 | LQNRRGLDLLFLKEGGLC | Q8AGX8 | Env | Python molurus |
| | | | | | | | endogenous |
| | | | | | | | retrovirus. |
| Q8AGX9_VVVVV | 467 | 550 | SEQ ID NO: 256 | LQNRRGLDLLFLKEGGLC | Q8AGX9 | Env | Python molurus |
| | | | | | | | endogenous |
| | | | | | | | retrovirus. |
| Q8B9S1_VVVVV | 554 | 648 | SEQ ID NO: 257 | ILNRKAIDFLLQRWGGTC | Q8B9S1 | Envelope | Zaire ebolavirus |
| | | | | | | glycoprotein | (strain Mayinga-76) |
| | | | | | | | (ZEBOV) (Zaire |
| | | | | | | | Ebola virus) |
| Q88141_MOUSE | 467 | 546 | SEQ ID NO: 258 | LQNRRALDLITAEKGGTC | Q8BI41 | 15 days embryo | Mus musculus |
| | | | | | | head | (Mouse). |
| | | | | | | cDNA Envelope | |
| | | | | | | glycoprotein | |
| | | | | | | syncytin-B) | |
| Q88U01_MOUSE | 461 | 554 | SEQ ID NO: 259 | LRNQREQDFQSLQQDGLC | Q8BUO1 | 2 days neonate | Mus musculus |
| | | | | | | thymus thymic | (Mouse). |
| | | | | | | cells cDNA | |
| Q8J4V5_VVVVV | 515 | 569 | SEQ ID NO: 260 | LQNRRGLDLLFLREGGLC | Q8J4V5 | Env protein | Porcine |
| | | | | | | | endogenous |
| | | | | | | | retrovirus B. |
| Q8J4V7_VVVVV | 518 | 572 | SEQ ID NO: 261 | LQNRRGLDLLFLKEGGLC | Q8J4V7 | Env protein | Porcine |
| | | | | | | | endogenous |
| | | | | | | | retrovirus A. |
| Q8JEM7_VVVVV | 518 | 572 | SEQ ID NO: 262 | LQNRRGLDLLFLKEGGLC | Q8JEM7 | Envelope | Porcine |
| | | | | | | glycoprotein | endogenous |
| | | | | | | | retrovirus. |
| Q8JGM1_CHICK | 264 | 372 | SEQ ID NO: 263 | LQNRMALDLLTAKEGGVC | Q8JGM1 | Female expressed | Gallus gallus |
| | | | | | | transcript 1 | (Chicken). |
| Q8JIZ0_BRARE | 470 | 543 | SEQ ID NO: 264 | IQNRLALDMLLSERGGVC | Q8JIZO | Envelope protein | Brachydanio rerio |
| | | | | | | | (Zebrafish) (Danio |
| | | | | | | | rerio). |
| Q8JPX8_VVVVV | 550 | 649 | SEQ ID NO: 265 | LLNRKAIDFLLQRWGGTC | Q66799 | Envelope protein | Reston ebolavirus |
| | | | | | | | (strain Reston-89) |
| | | | | | | | (REBOV) (Reston |
| | | | | | | | Ebola virus) |
| Q8JS62_VVVVV | 554 | 648 | SEQ ID NO: 266 | ILNRKAIDFLLQRWGGTC | Q05320 | Envelope protein | Zaire ebolavirus |
| | | | | | | | (strain Mayinga-76) |
| | | | | | | | (ZEBOV) (Zaire |
| | | | | | | | Ebola virus) |
| Q8K030_MOUSE | 525 | 594 | SEQ ID NO: 267 | LQNRRGLDLLFLKEGGLC | Q8K030 | BC035947 protein | Mus musculus |
| | | | | | | | (Mouse). |
| Q8MIB6_PANTR | 433 | 518 | SEQ ID NO: 268 | MQNRRALDLLTADKGGTC | Q8MIB6 | ERV-F(c)1 provirus | Pan troglodytes |
| | | | | | | ancestral Env | (Chimpanzee). |
| | | | | | | polyprotein | |
| | | | | | | precursor | |
| Q8NC12_HUMAN | 5 | 61 | SEQ ID NO: 269 | LQNRRGLDMLTAAQGGIC | Q8NC12 | CDNA FLJ90611 fis | Homo sapiens |
| | | | | | | | (Human). |
| Q8Q6U6_VVVVV | 518 | 572 | SEQ ID NO: 270 | LQNRRGLDLLFLKEGGLC | Q80606 | Envelope protein | Porcine |
| | | | | | | | endogenous |
| | | | | | | | retrovirus. |
| Q8Q6Y6_VVVVV | 518 | 570 | SEQ ID NO: 271 | LQNRRGLDLLFLKEGGLC | Q8Q6Y6 | Envelope glyco- | Porcine |
| | | | | | | protein | endogenous |
| | | | | | | | retrovirus. |
| Q8Q6Y7_VVVVV | 518 | 570 | SEQ ID NO: 272 | LQNRRGLDLLFLKEGGLC | Q8Q6Y7 | Envelope glyco- | Porcine |
| | | | | | | protein | endogenous |
| | | | | | | | retrovirus. |
| Q8Q6Y8_VVVVV | 518 | 570 | SEQ ID NO: 273 | LQNRRGLDLLFLKEGGLC | Q8Q6Y8 | Envelope glyco- | Porcine |
| | | | | | | protein | endogenous |
| | | | | | | | retrovirus. |
| Q8Q6Y9_VVVVV | 500 | 552 | SEQ ID NO: 274 | LQNRRGLDLLFLKEGGLC | Q8Q6Y9 | Envelope glyco- | Porcine |
| | | | | | | protein | endogenous |
| | | | | | | | retrovirus. |
| Q8Q6Z0_VVVVV | 512 | 564 | SEQ ID NO: 275 | LQNRRGLDLLFLKEGGLC | Q8Q6Z0 | Envelope glyco- | Porcine |
| | | | | | | protein | endogenous |
| | | | | | | | retrovirus. |
| Q8Q6Z1_VVVVV | 512 | 564 | SEQ ID NO: 276 | LQNRRGLDLLFLKEGGLC | Q8Q6Z1 | Envelope glyco- | Porcine |
| | | | | | | protein | endogenous |
| | | | | | | | retrovirus. |
| Q8QH10_BRARE | 471 | 542 | SEQ ID NO: 277 | IQNRLALDMLLSERGGVC | Q8QH10 | Envelope protein | Brachydanio rerio |
| | | | | | | | (Zebrafish) (Danio |
| | | | | | | | rerio). |
| Q8R067_MOUSE | 461 | 554 | SEQ ID NO: 278 | LRNQREQDFQSLQQDGLC | Q8R067 | DNA segment, Chr | Mus musculus |
| | | | | | | 17, human D6S56E 5 | (Mouse). |
| Q8UM95_VVVVV | 515 | 569 | SEQ ID NO: 279 | LQNRRGLDLLFLREGGLC | Q8UM95 | Env protein | Porcine |
| | | | | | | | endogenous |
| | | | | | | | retrovirus. |
| Q8UM98_VVVVV | 518 | 600 | SEQ ID NO: 280 | LQNRRGLDLLFLKEGGLC | Q8UM98 | Env protein | Porcine |
| | | | | | | (Envelope | endogenous |
| | | | | | | protein) | retrovirus. |
| Q8UMP4_VVVVV | 515 | 569 | SEQ ID NO: 281 | LQNRRGLDLLFLREGGLC | Q8UMP4 | Env | Porcine |
| | | | | | | | endogenous |
| | | | | | | | retrovirus. |
| Q8UMZ9_MLVMO | 523 | 606 | SEQ ID NO: 282 | LQNRRGLDLLFLKEGGLC | Q8UMZ9 | GPr80 glycosylated | Moloney murine |
| | | | | | | envelope | leukemia virus |
| | | | | | | polyprotein | (MoMLV). |
| Q900A0_VVVVV | 346 | 439 | SEQ ID NO: 283 | AQNRRGLDLXFWEQGGLC | Q900A0 | Envelope | Human T- |
| | | | | | | glycoprotein | lymphotropic virus |
| | | | | | | | 1. |
| Q905D5_HTLV2 | 373 | 435 | SEQ ID NO: 284 | -QNRRGLDLLFWEQGGLC | Q905D5 | Envelope | Human T-cell |
| | | | | | | glycoprotein | leukemia virus 2 |
| | | | | | | | (HTLV-2). |
| Q909T7_VVVVV | 346 | 439 | SEQ ID NO: 285 | AQNRRGLDLXFWEQGGLC | Q909T7 | Envelope | Human T- |
| | | | | | | glycoprotein | lymphotropic virus |
| | | | | | | | 1. |
| Q909T8_VVVVV | 346 | 439 | SEQ ID NO: 286 | AQNRRGLDLXFWEQGGLC | Q909T8 | Envelope | Human T- |
| | | | | | | glycoprotein | lymphotropic virus |
| | | | | | | | 1. |
| Q909T9_VVVVV | 346 | 439 | SEQ ID NO: 287 | AQNRRGLDLXFWEQGGLC | Q909T9 | Envelope | Human T- |
| | | | | | | glycoprotein | lymphotropic virus |
| | | | | | | | 1. |
| Q909U0_VVVVV | 346 | 439 | SEQ ID NO: 288 | AQNRRGLDLLFWEQGGLC | Q909U0 | Envelope | Human T- |
| | | | | | | glycoprotein | lymphotropic virus |
| | | | | | | | 1. |
| Q909U1_VVVVV | 346 | 439 | SEQ ID NO: 289 | AQNRRGLDLXFWEQGGLC | Q909U1 | Envelope | Human T- |
| | | | | | | glycoprotein | lymphotropic virus |
| | | | | | | | 1. |
| Q909U2_VVVVV | 346 | 439 | SEQ ID NO: 290 | AQNRRGLDLLFWEQGGLC | Q909U2 | Envelope | Human T- |
| | | | | | | glycoprotein | lymphotropic virus |
| | | | | | | | 1. |
| Q909U3_VVVVV | 346 | 408 | SEQ ID NO: 291 | AQNRRGLDLLFWEQGGLC | Q909U3 | Envelope | Human T- |
| | | | | | | glycoprotein | lymphotropic virus |
| | | | | | | | 1. |
| Q909U4_VVVVV | 377 | 439 | SEQ ID NO: 292 | -QNRRGLDLLFWEQGGLC | Q909U4 | Envelope | Human T- |
| | | | | | | glycoprotein | lymphotropic virus |
| | | | | | | | 1. |
| Q909U5_VVVVV | 346 | 439 | SEQ ID NO: 293 | AQNRRGLDLXFWEQGGLC | Q909U5 | Envelope | Human T- |
| | | | | | | glycoprotein | lymphotropic virus |
| | | | | | | | 1. |
| Q90AE9_FLV | 519 | 602 | SEQ ID NO: 294 | LQNRRGLDILFLQEGGLC | Q90AE9 | Env polyprotein | Feline leukemia |
| | | | | | | | virus. |
| Q90LU1_ALV | 402 | 514 | SEQ ID NO: 295 | LQNRAAIDFLLLAQGHGC | Q90LU1 | Envelope proteins | Avian leukosis virus |
| | | | | | | | (ALV). |
| Q90LX2_VVVVV | 448 | 530 | SEQ ID NO: 296 | LQNKKGLDLLFLKKRRLC | Q90LX2 | Envelope protein | Porcine |
| | | | | | | | endogenous type C |
| | | | | | | | retrovirus. |
| Q90LX3_VVVVV | 497 | 549 | SEQ ID NO: 297 | LQNRRGLDLLFLKEGGLC | Q90LX3 | Envelope protein | Porcine |
| | | | | | | | endogenous type C |
| | | | | | | | retrovirus. |
| Q90LX4_VVVVV | 497 | 549 | SEQ ID NO: 298 | LQNRRGLDLLFLKEGGLC | Q90LX4 | Envelope protein | Porcine |
| | | | | | | | endogenous type C |
| | | | | | | | retrovirus. |
| Q90LX5_VVVVV | 497 | 549 | SEQ ID NO: 299 | LQNRRGLDLLFLKEGGLC | Q90LX5 | Envelope protein | Porcine |
| | | | | | | | endogenous type C |
| | | | | | | | retrovirus. |
| Q90RI4_FLV | 500 | 582 | SEQ ID NO: 300 | LQNRRGLDILFLQGGGLC | Q90R14 | Envelope protein | Feline leukemia |
| | | | | | | | virus. |
| Q90RL3_VVVVV | 524 | 607 | SEQ ID NO: 301 | LQNRRGLDLLFLKEGGLC | Q90RL3 | Env protein | Murine leukemia |
| | | | | | | | virus. |
| Q90RL5_VVVVV | 515 | 569 | SEQ ID NO: 302 | LQNRRGLDLLFLREGGLC | Q90RL5 | Envelope | Porcine |
| | | | | | | | endogenous type C |
| | | | | | | | retrovirus. |
| Q90RL8_VVVVV | 518 | 600 | SEQ ID NO: 303 | LQNRRGLDLLFLKEGGLC | Q90RL8 | Envelope | Porcine |
| | | | | | | | endogenous type C |
| | | | | | | | retrovirus. |
| Q913A3_VVVVV | 554 | 648 | SEQ ID NO: 304 | ILNRKAIDFLLQRWGGTC | O11457 | Envelope protein | Zaire ebolavirus |
| | | | | | | | (strain Gabon-941 |
| | | | | | | | (ZEBOV) (Zaire |
| | | | | | | | Ebola virus) |
| Q91DD8_VVVVV | 550 | 649 | SEQ ID NO: 305 | LLNRKAIDFLLQRWGGTC | Q91DD8 | Envelope protein | Reston ebolavirus |
| | | | | | | | (strain Philippines- |
| | | | | | | | 96) (REBOV) |
| | | | | | | | (Reston Ebola virus) |
| Q91UZ6_MOUSE | 461 | 554 | SEQ ID NO: 306 | LRNQREQDFQSLQQDGLC | Q91UZ6 | Viral envelope | Mus musculus |
| | | | | | | protein G7e | (Mouse). |
| Q91Y75_MUSMC | 524 | 607 | SEQ ID NO: 307 | LQNRRGLDLLFLKEGGLC | Q91Y75 | Envelope | Mus musculus |
| | | | | | | | castaneus |
| | | | | | | | (Southeastern Asian |
| | | | | | | | house mouse). |
| Q96MK7_HUMAN | 107 | 169 | SEQ ID NO: 308 | MNNRLALDYLLAEQGGVC | Q96MK7 | CDNA FLJ32214 fis, | Homo sapiens |
| | | | | | | clone PLACE6003705 | (Human). |
| Q98654_VVVVV | 416 | 490 | SEQ ID NO: 309 | LQNRRGLDLLTAEQGGIC | Q98654 | Envelope protein | RD114 retrovirus. |
| Q98WV9_ALV | 440 | 553 | SEQ ID NO: 310 | LQNRAAIDFLLLAHGHGC | Q98WV9 | Pr57 env | Avian leukosis virus |
| | | | | | | polyprotein | (ALV). |
| Q98WW1_ALV | 442 | 555 | SEQ ID NO: 311 | LQNRAAIDFLLLAHGHGC | Q98WW1 | Pr57 env | Avian leukosis virus |
| | | | | | | polyprotein | (ALV). |
| Q99043_VVVVV | 472 | 580 | SEQ ID NO: 312 | LQNRRGLDLLFLKEGGLC | Q99043 | Envelope protein | Xenotropic murine |
| | | | | | | | leukemia virus. |
| Q991W9_VVVVV | 368 | 444 | SEQ ID NO: 313 | LQNRRALDLLTAKRGGTC | Q991W9 | Recombinant | Multiple sclerosis |
| | | | | | | envelope protein | associated |
| | | | | | | | retrovirus element. |
| Q992L2_VVVVV | 476 | 556 | SEQ ID NO: 314 | LQNRRGLDLLFLKEGGLC | Q992L2 | Envelope glyco- | Mus cervicolor |
| | | | | | | protein | popaeus |
| | | | | | | | endogenous virus. |
| Q90576_MOUSE | 189 | 247 | SEQ ID NO: 315 | LQNRQGLDVLSAKEGGLC | Q9D576 | Adult male testis | Mus musculus |
| | | | | | | | (Mouse). |
| Q9DKR5_HTLV2 | 373 | 435 | SEQ ID NO: 316 | -QNRRGLDLLFWEQGGLC | Q9DKR5 | Env | Human T-cell |
| | | | | | | | leukemia virus 2 |
| | | | | | | | (HTLV-2). |
| Q9DKR9_HTLV2 | 373 | 435 | SEQ ID NO: 317 | -QNRRGLDLLFWEQGGLC | Q9DKR9 | Env | Human T-cell |
| | | | | | | | leukemia virus 2 |
| | | | | | | | (HTLV-2). |
| Q9DLK2_ALV | 405 | 517 | SEQ ID NO: 318 | LQNRAAIDFLLLAQGHGC | Q9DLK2 | Envelope protein | Avian leukosis virus |
| | | | | | | | (ALV). |
| Q9DLK3_ALV | 405 | 517 | SEQ ID NO: 319 | LQNRAAIDFLLLAQGHGC | Q9DLK3 | Envelope protein | Avian leukosis virus |
| | | | | | | | (ALV). |
| Q9DLK4_ALV | 402 | 514 | SEQ ID NO: 320 | LQNRAAIDFLLLAQGHGC | Q9DLK4 | Envelope protein | Avian leukosis virus |
| | | | | | | | (ALV). |
| Q9DLK5_ALV | 404 | 516 | SEQ ID NO: 321 | LQNRAAIDFLLLAQGHGC | Q9DLK5 | Envelope protein | Avian leukosis virus |
| | | | | | | | (ALV). |
| Q9DQ21_VVVVV | 494 | 577 | SEQ ID NO: 322 | LQNRRGLDLLFLKEGGLC | Q9DQ21 | Envelope protein | Murine leukemia |
| | | | | | | | virus. |
| Q9DQ22_VVVVV | 472 | 580 | SEQ ID NO: 323 | LQNRRGLDLLFLKEGGLC | Q9DQ22 | Envelope protein | Murine leukemia |
| | | | | | | | virus. |
| Q9DQ23_VVVVV | 533 | 616 | SEQ ID NO: 324 | LQNRRGLDLLFLKEGGLC | Q9DQ23 | Envelope protein | Murine leukemia |
| | | | | | | | virus. |
| Q9DQ24_VVVVV | 524 | 607 | SEQ ID NO: 325 | LQNRRGLDLLFLKEGGLC | Q9DQ24 | Envelope protein | Murine leukemia |
| | | | | | | | virus. |
| Q9E7M0_VVVVV | 472 | 580 | SEQ ID NO: 326 | LQNRRGLDLLFLKEGGLC | Q9E7M0 | Putative envelope | DG-75 Murine |
| | | | | | | polyprotein | leukemia virus. |
| Q9GLF7_TRIVU | 416 | 504 | SEQ ID NO: 327 | LQNRRGLDLLTAEQGGIC | Q9GLF7 | Envelope protein | Trichosurus |
| | | | | | | | vulpecula (Brush- |
| | | | | | | | tailed possum). |
| Q9IGU2_FOWPV | 447 | 525 | SEQ ID NO: 328 | LQNRRGLDLLTAEQGGIC | Q9IGU2 | Envelope | Fowlpox virus |
| | | | | | | glycoprotein | (FPV). |
| Q9IUF0_VVVVV | 515 | 569 | SEQ ID NO: 329 | LQNRRGLDLLFLREGGLC | Q9IUF0 | Envelope protein | Porcine |
| | | | | | | | endogenous |
| | | | | | | | retrovirus. |
| Q9IUF3_VVVVV | 512 | 566 | SEQ ID NO: 330 | LQNRRGLDLLFLREGGLC | Q9IUF3 | Envelope protein | Porcine |
| | | | | | | | endogenous |
| | | | | | | | retrovirus. |
| Q9IUF6_VVVVV | 518 | 600 | SEQ ID NO: 331 | LQNRRGLDLLFLKEGGLC | Q9IUF6 | Envelope protein | Porcine |
| | | | | | | | endogenous |
| | | | | | | | retrovirus. |
| Q9IUF7_VVVVV | 518 | 572 | SEQ ID NO: 332 | LQNRRGLDLLFLKEGGLC | Q9IUF7 | Envelope protein | Porcine |
| | | | | | | | endogenous |
| | | | | | | | retrovirus. |
| Q9IWU7_VVVVV | 346 | 408 | SEQ ID NO: 333 | AQNRRGLDLLFWEQGGLC | Q9IWU7 | Envelope protein | Human T- |
| | | | | | | | lymphotropic virus |
| | | | | | | | 1. |
| Q9J056_VVVVV | 346 | 439 | SEQ ID NO: 334 | AQNRRGLDLLFWEQGGLC | Q9J056 | Envelope | Human T- |
| | | | | | | glycoprotein | lymphotropic virus |
| | | | | | | | 1. |
| Q9N2J9_VVVVV | 436 | 497 | SEQ ID NO: 335 | LQNRQGLDLLTAEKGGLC | Q9N2J9 | HERV-H_3q26 | Homo sapiens |
| | | | | | | provirus | (Human). |
| | | | | | | ancestral Env | |
| | | | | | | polyprotein | |
| | | | | | | precursor | |
| Q9N2K0_VVVVV | 436 | 501 | SEQ ID NO: 336 | LQNRRGLDLLTAEKGGLC | Q9N2K0 | HERV-H_2q24.3 | Homo sapiens |
| | | | | | | provirus | (Human). |
| | | | | | | ancestral Env | |
| Q9NRZ2_HUMAN | 368 | 447 | SEQ ID NO: 337 | LRNRRALDLLTAERGGTC | Q9UQF0 | HERV-W_7q21.2 | Homo sapiens |
| | | | | | | provirus | (Human) |
| | | | | | | ancestral Env | polyprotein |
| Q9NZG3_HUMAN | 368 | 447 | SEQ ID NO: 338 | LQNRRALDLLTAERGGTC | Q9UQF0 | HERV-W_7q21.2 | Homo sapiens |
| | | | | | | provirus ancestral | (Human) |
| | | | | | | Env polyprotein | |
| Q9PWB9_CHICK | 937 | 1049 | SEQ ID NO: 339 | LQNRAVIDFLLLAQGHGC | Q9PWB9 | Gag/env fusion | Gallus gallus |
| | | | | | | protein | (Chicken). |
| Q9PY03_VVVVV | 346 | 439 | SEQ ID NO: 340 | AQNRRRLDLLFWEQGGLC | Q9PY03 | Envelope | Human T. |
| | | | | | | glycoprotein(GP21, | lymphotropic virus |
| | | | | | | GP46) | 1. |
| Q9Q1X3_VVVVV | 2234 | 2288 | SEQ ID NO: 341 | LQNRRGLDLLFLREGGLC | Q9Q1X3 | Type C proviral gag, | Porcine |
| | | | | | | pol and env genes | endogenous |
| | | | | | | and LTR (class B, | retrovirus. |
| | | | | | | clone 43) | |
| Q9Q1X4_VVVVV | 2236 | 2290 | SEQ ID NO: 342 | LQNRRGLDLLFLKEGGLC | Q9Q1X4 | Type C proviral gag, | Porcine |
| | | | | | | pol and env genesand | endogenous |
| | | | | | | LTR (class A, clone | retrovirus. |
| | | | | | | 42) | |
| Q9Q1X5_VVVVV | 2234 | 2288 | SEQ ID NO: 343 | LQNRRGLDLLFLREGGLC | Q9Q1X5 | Type C proviral gag, | Porcine |
| | | | | | | pol and env genes | endogenous |
| | | | | | | and LTR (class B, | retrovirus. |
| | | | | | | clone 33) | |
| Q9Q9A5_VVVVV | 542 | 625 | SEQ ID NO: 344 | LQNRRGLDLLFLKEGGLC | Q9Q9A5 | Putative envelope | Murine leukemia |
| | | | | | | polyprotein | virus. |
| Q9Q9X3_VVVVV | 518 | 570 | SEQ ID NO: 345 | LQNRRGLDLLFLKEGGLC | Q9Q9X3 | Envelope glyco- | Porcin |
| | | | | | | proteine | endogenous type C |
| | | | | | | | retrovirus. |
| Q9QME4_VVVVV | 959 | 1071 | SEQ ID NO: 346 | LQNRAAIDFLLLAQGHGC | Q9QME4 | Gag-env fusion | Avian endogenous |
| | | | | | | protein | retrovirus EAV-HP. |
| Q9TTC0_PHACI | 518 | 596 | SEQ ID NO: 347 | LQNRRGLDLLFLKEGGLC | Q9TTC0 | Envelope glyco- | Koala retrovirus |
| | | | | | | protein | (KoRV) |
| Q9UNM3_HUMAN | 436 | 501 | SEQ ID NO: 348 | LQNRRGLDLLTAEKGGLC | Q9UNM3 | Envelope glyco- | Homo sapiens |
| | | | | | | protein | (Human) |
| Q9UQF0_HUMAN | 368 | 447 | SEQ ID NO: 349 | LQNRRALDLLTAERGGTC | Q9UQF0 | Envelope glyco- | Homo sapiens |
| | | | | | | protein | (Human) |
| Q9WHJ7_FRMCV | 493 | 576 | SEQ ID NO: 350 | LQNRRGLDLLFLKEGGLC | Q9WHJ7 | Envelope protein | Friend mink cell |
| | | | | | | | focus-inducing |
| | | | | | | | virus. |
| Q9WHV5_VVVVV | 468 | 576 | SEQ ID NO: 351 | LQNRRGLDLLFLKEGGLC | Q9WHV5 | Envelope protein | Murine leukemia |
| | | | | | | | virus. |
| Q9WI17_HTLV2 | 373 | 435 | SEQ ID NO: 352 | -QNRRGLDLLFWEQGGLC | Q9WI17 | Env | Human T-cell |
| | | | | | | | leukemia virus 2 |
| | | | | | | | (HTLV-2). |
| Q9WLJ4_VVVVV | 472 | 580 | SEQ ID NO: 353 | LQNRRGLDLLFLKEGGLC | Q9WLJ4 | Envelope protein | Murine leukemia |
| | | | | | | | virus. |
| Q9WS53_VVVVV | 1075 | 1168 | SEQ ID NO: 354 | AQNRRGLDLLFWEQGGLC | Q9WS53 | Reverse | Simian T- |
| | | | | | | transcriptase/ | lymphotropic virus |
| | | | | | | envelope protein | 1. |
| Q9WS57_HTLV2 | 373 | 435 | SEQ ID NO: 355 | -QNRRGLDLLFWEQGGLC | Q9WS57 | Envelope protein | Human T-cell |
| | | | | | | | leukemia virus 2 |
| | | | | | | | (HTLV-2). |
| Q9XSY3_FELCA | 515 | 598 | SEQ ID NO: 356 | LQNRRGLDLLFLQEGGLC | Q9XSY3 | Envelope protein | Felis silvestris |
| | | | | | | | catus (Cat). |
| Q9YWL9_VVVVV | 528 | 611 | SEQ ID NO: 357 | LQNRRGLDLLFLKEGGLC | Q9YWL9 | Envelope protein | Simian sarcoma- |
| | | | | | | | associated virus. |
| Q9YWM0_GALV | 529 | 607 | SEQ ID NO: 358 | LQNRRGLDLLFLKEGGLC | Q9YWM0 | Envelope protein | Gibbon ape |
| | | | | | | | leukemia virus |
| | | | | | | | (GALV). |
| Q9YWM1_GALV | 541 | 623 | SEQ ID NO: 359 | LQNRRGLDLLFLKEGGLC | Q9YWM1 | Envelope protein | Gibbon ape |
| | | | | | | | leukemia virus |
| | | | | | | | (GALV). |
| Q9YWM2_GALV | 545 | 627 | SEQ ID NO: 360 | LQNRRGLDLLFLKEGGLC | Q9YWM2 | Envelope protein | Gibbon ape |
| | | | | | | | leukemia virus |
| | | | | | | | (GALV). |
| Q9YWM3_GALV | 547 | 622 | SEQ ID NO: 361 | LQNRRGLDLLFLKEGGLC | Q9YWM3 | Envelope protein | Gibbon ape |
| | | | | | | | leukemia virus |
| | | | | | | | (GALV). |
| Q9YYS3_VVVVV | 527 | 610 | SEQ ID NO: 362 | LQNRRGLDLLFLKEGGLC | Q9YYS3 | Envelope | Murine leukemia |
| | | | | | | polypeptide | virus. |
| VGP_EBOEC | 554 | 648 | SEQ ID NO: 363 | ILNRKAIDFLLQRWGGTC | P87671 | Envelope | Zaire ebolavirus |
| | | | | | | glycoprotein | (strain Eckron-76) |
| | | | | | | precursor | (ZEBOV) (Zaire |
| | | | | | | (GP1,2) (GP) | Ebola virus). |
| VGP_EBOG4 | 554 | 648 | SEQ ID NO: 364 | ILNRKAIDFLLQRWGGTC | O11457 | Envelope | Zaire ebolavirus |
| | | | | | | glycoprotein | (strain Gabon-94) |
| | | | | | | precursor | (ZEBOV) (Zaire |
| | | | | | | (GP1,2) (GP) | Ebola virus). |
| VGP_EBOIC | 550 | 648 | SEQ ID NO: 365 | ILNRKAIDFLLQRWGGTC | Q66810 | Envelope | Ivory Coast |
| | | | | | | glycoprotein | ebolavirus (strain |
| | | | | | | precursor | Cote d'Ivoire-94) |
| | | | | | | (GP1,2) (GP) | (CIEBOV) (Cote |
| | | | | | | | d'Ivoire Ebola |
| | | | | | | | virus). |
| VGP_EBORE | 550 | 649 | SEQ ID NO: 366 | LLNRKAIDFLLQRWGGTC | Q91DD8 | Envelope | Reston ebolavirus |
| | | | | | | glycoprotein | (strain Philippines- |
| | | | | | | precursor | 96) (REBOV) |
| | | | | | | (GP1,2) (GP) | (Reston Ebola |
| | | | | | | | virus). |
| VGP_EBORR | 550 | 649 | SEQ ID NO: 367 | LLNRKAIDFLLQRWGGTC | Q66799 | Envelope | Reston ebolavirus |
| | | | | | | glycoprotein | (strain Reston-89) |
| | | | | | | precursor | (REBOV) (Reston |
| | | | | | | (GP1,2) (GP) | Ebola virus). |
| VGP_EBORS | 550 | 649 | SEQ ID NO: 368 | LLNRKAIDFLLQRWGGTC | Q89853 | Envelope | Reston ebolavirus |
| | | | | | | glycoprotein | (strain |
| | | | | | | precursor | Siena/Philippine-92) |
| | | | | | | (GP1,2) (GP) | (REBOV) (Reston |
| | | | | | | | Ebola virus). |
| VGP_EBOSB | 547 | 648 | SEQ ID NO: 369 | ILNRKAIDFLLRRWGGTC | Q66814 | Envelope | Sudan ebolavirus |
| | | | | | | glycoprotein | (strain Boniface-76) |
| | | | | | | precursor | (SEBOV) (Sudan |
| | | | | | | (GP1,2) (GP) | Ebola virus). |
| VGP_EBOSM | 547 | 648 | SEQ ID NO: 370 | ILNRKAIDFLLRRWGGTC | Q66798 | Envelope | Sudan ebolavirus |
| | | | | | | glycoprotein | (strain Maleo-79) |
| | | | | | | precursor | (SEBOV) (Sudan |
| | | | | | | (GP1,2) (GP) | Ebola virus). |
| VGP_EBOZ5 | 554 | 648 | SEQ ID NO: 371 | ILNRKAIDFLLQRWGGTC | P87666 | Envelope | Zaire ebolavirus |
| | | | | | | glycoprotein | (strain Kikwit-95) |
| | | | | | | precursor | (ZEBOV) (Zaire |
| | | | | | | (GP1,2) (GP) | Ebola virus). |
| VGP_EBOZM | 554 | 648 | SEQ ID NO: 372 | ILNRKAIDFLLQRWGGTC | Q05320 | Envelope | Zaire ebolavirus |
| | | | | | | glycoprotein | (strain Mayinga-76) |
| | | | | | | precursor | (ZEBOV) (Zaire |
| | | | | | | (GP1,2) (GP) | Ebola virus). |
| VGP_MABVM | 546 | 644 | SEQ ID NO: 373 | LINRHAIDFLLTRWGGTC | Q05320 | Envelope | Zaire ebolavirus |
| | | | | | | glycoprotein | (strain Mayinga-76) |
| | | | | | | precursor | (ZEBOV) (Zaire |
| | | | | | | (GP1,2) (GP) | Ebola virus). |
| VGP_MABVP | 546 | 644 | SEQ ID NO: 374 | LINRHAIDFLLTRWGGTC | Q05320 | Envelope | Zaire ebolavirus |
| | | | | | | glycoprotein | (strain Mayinga-76) |
| | | | | | | precursor | (ZEBOV) (Zaire |
| | | | | | | (GP1,2) (GP) | Ebola virus). |
| VGP_MABVM | 546 | 644 | SEQ ID NO: 375 | LINRHAIDFLLTRWGGTC | P35253 | Structural | Lake Victoria |
| | | | | | | glycoprotein | marburgvirus |
| | | | | | | precursor | (strain |
| | | | | | | (Virion spike | Musoke-80) |
| | | | | | | glycoprotein) | |
| VGP_MABVP | 546 | 644 | SEQ ID NO: 376 | LINRHAIDFLLTRWGGTC | P35254 | Structural | Lake Victoria |
| | | | | | | glycoprotein | marburgvirus |
| | | | | | | precursor | (strain |
| | | | | | | (Virion spike | Popp-67) |
| | | | | | | glycoprotein) | |

### REFERENCES

Benit L, Dessen P and Heidmann T. Identification, phylogeny, and evolution of retroviral elements based on their envelope genes. J Virol 2001; 75:11709-19
Kelley L A, MacCallum R M and Sternberg M J. Enhanced genome annotation using structural profiles in the program 3D-PSSM. J Mol Biol 2000; 299:499-520
Schnittler H J, Feldmann H. Viral hemorrhagic fever—a vascular disease? Thromb Haemost 2003; 89:967-72
Geisbert T W, Hensley L E, Larsen T, et al. Pathogenesis of Ebola hemorrhagic fever in cynomolgus macaques: evidence that dendritic cells are early and sustained targets of infection. Am J Pathol 2003; 163:2347-70
Feldmann H, Jones S, Klenk H D and Schnittler H J. Ebola virus: from discovery to vaccine. Nat Rev Immunol 2003; 3:677-85
Feldmann H, Bugany H, Mahner F, Klenk H D, Drenckhahn D and Schnittler H J. Filovirus-induced endothelial leakage triggered by infected monocytes/macrophages. J Virol 1996; 70:2208-14
Baize S, Leroy E M, Mavoungou E and Fisher-Hoch S P. Apoptosis in fatal Ebola infection. Does the virus toll the bell for immune system? Apoptosis 2000; 5:5-7
Hensley L E, Young H A, Jahrling P B and Geisbert T W. Proinflammatory response during Ebola virus infection of primate models: possible involvement of the tumor necrosis factor receptor superfamily. Immunol Lett 2002; 80:169-79
Stroher U, West E, Bugany H, Klenk H D, Schnittler H J and Feldmann H. Infection and activation of monocytes by Marburg and Ebola viruses. J Virol 2001; 75:11025-33
Feldmann H, Volchkov V E, Volchkova V A and Klenk H D. The glycoproteins of Marburg and Ebola virus and their potential roles in pathogenesis. Arch Virol Suppl 1999; 15:159-69
Bray M, Davis K, Geisbert T, Schmaljohn C and Huggins J. A mouse model for evaluation of prophylaxis and therapy of Ebola hemorrhagic fever. J Infect Dis 1998; 178:651-61
Connolly B M, Steele K E, Davis K J, et al. Pathogenesis of experimental Ebola virus infection in guinea pigs. J Infect Dis 1999; 179 Suppl 1:S203-17
Volchkov V E, Blinov V M and Netesov S V. The envelope glycoprotein of Ebola virus contains an immunosuppressive-like domain similar to oncogenic retroviruses. FEBS Lett 1992; 305:181-4
Bukreyev A, Volchkov V E, Blinov V M and Netesov S V. The GP-protein of Marburg virus contains the region similar to the ‘immunosuppressive domain’ of oncogenic retrovirus P15E proteins. FEBS Lett 1993; 323:183-7
Good R A, Haraguchi S, Lorenz E and Day N K. In vitro immunomodulation and in vivo immunotherapy of retrovirus-induced immunosuppression. Int J Immunopharmacol 1991; 13 Suppl 1:1-7
Haraguchi S, Liu W T, Cianciolo G J, Good R A and Day N K. Suppression of human interferon-gamma production by a 17 amino acid peptide homologous to the transmembrane envelope protein of retroviruses: evidence for a primary role played by monocytes. Cell Immunol 1992; 141:388-97
Haraguchi S, Good R A, James-Yarish M, Cianciolo G J and Day N K Differential modulation of Th1- and Th2-related cytokine mRNA expression by a synthetic peptide homologous to a conserved domain within retroviral envelope protein. Proc Natl Acad Sci USA 1995; 92:3611-5
Haraguchi S, Good R A, James-Yarish M, Cianciolo G J and Day N K. Induction of intracellular cAMP by a synthetic retroviral envelope peptide: a possible mechanism of immunopathogenesis in retroviral infections. Proc Natl Acad Sci USA 1995; 92:5568-71
Haraguchi S, Good R A, Cianciolo G J, James-Yarish M and Day N K. Transcriptional down-regulation of tumor necrosis factor-alpha gene expression by a synthetic peptide homologous to retroviral envelope protein. J Immunol 1993; 151:2733-41
Haraguchi S, Good R A, Cianciolo G J and Day N K. A synthetic peptide homologous to retroviral envelope protein down-regulates TNF-alpha and IFN-gamma mRNA expression. J Leukoc Biol 1992; 52:469-72
Ogasawara M, Haraguchi S, Cianciolo G J, Mitani M, Good R A and Day N K. Inhibition of murine cytotoxic T lymphocyte activity by a synthetic retroviral peptide and abrogation of this activity by IL. J Immunol 1990; 145:456-62
Ogasawara M, Cianciolo G J, Snyderman R, Mitani M, Good R A and Day N K. Human IFN-gamma production is inhibited by a synthetic peptide homologous to retroviral envelope protein. J Immunol 1988; 141:614-9
Ogasawara M, Cianciolo G J, Mitani M, Kizaki T, Good R A and Day N K. The suppressive effect of a synthetic retroviral peptide on the human IFN gamma production is abrogated by the combined stimulation with IL-1 and IL-2. Cancer Detect Prev 1991; 15:205-9
Naito T, Ogasawara H, Kaneko H, et al. Immune abnormalities induced by human endogenous retroviral peptides: with reference to the pathogenesis of systemic lupus erythematosus. J Clin Immunol 2003; 23:371-6
Cianciolo G J, Bogerd H and Snyderman R. Human retrovirus-related synthetic peptides inhibit T lymphocyte proliferation. Immunol Lett 1988; 19:7-13
Denner J, Persin C, Vogel T, Haustein D, Norley S and Kurth R. The immunosuppressive peptide of HIV-1 inhibits T and B lymphocyte stimulation. J Acquir Immune Defic Syndr Hum Retrovirol 1996; 12:442-50
Denner J, Norley S and Kurth R. The immunosuppressive peptide of HIV-1: functional domains and immune response in AIDS patients. Aids 1994; 8:1063-72
Haraguchi S, Cianciolo G J, Good R A, James-Yarish M, Brigino E and Day N K. Inhibition of interleukin-2 and interferon-gamma by an HIV-1 Nef-encoded synthetic peptide. Aids 1998; 12:820-3
Haraguchi S, Good R A, Cianciolo G J, Engelman R W and Day N K. Immunosuppressive retroviral peptides: immunopathological implications for immunosuppressive influences of retroviral infections. J Leukoc Biol 1997; 61:654-66
Huang S S, Huang J S. A pentacosapeptide (CKS-25) homologous to retroviral envelope proteins possesses a transforming growth factor-beta activity. J Biol Chem 1998; 273:4815-8
Ruegg C L, Strand M. Identification of a decapeptide region of human interferon-alpha with antiproliferative activity and homology to an immunosuppressive sequence of the retroviral transmembrane protein P15E. J Interferon Res 1990; 10:621-6
Wei E T, Thomas H A. Anti-inflammatory peptide agonists. Annu Rev Pharmacol Toxicol 1993; 33:91-108
Takahashi A, Day N K, Luangwedchakam V, Good R A and Haraguchi S. A retroviral-derived immunosuppressive peptide activates mitogen-activated protein kinases. J Immunol 2001; 166:6771-5
Luangwedchakam V, Day N K, Hitchcock R, et al. A retroviral-derived peptide phosphorylates protein kinase D/protein kinase Cmu involving phospholipase C and protein kinase C. Peptides 2003; 24:631-7
Peters, C. I, and LeDuc, J. W. (1999) An introduction to Ebola: the virus and the disease. J Infect Dis 179 Suppl 1, ix-xvi.
Jahrling, P. B., Geisbert, T. W., Jaax, N. K., Hanes, M. A., Ksiazek, T. G., and Peters, C. J. (1996) Experimental infection of cynomolgus macaques with Ebola-Reston filovirases from the 1989-1990 U.S. epizootic. Arch Virol Suppl 11, 115-134
Feldmann, H., and Klenk, H. D. (1996) Marburg and Ebola viruses. Adv Virus Res 47, 1-52
Basler, C. E, Wang, X., Muhlberger, E., Volchkov, V., Paragas, J., Klenk, H. D., Garcia-Sastre, A., and Palese, P. (2000) The Ebola virus VP35 protein functions as a type IIFN antagonist. Proc Natl Acad Sci USA91, 12289-12294
Geisbert, T. W., Hensley, L. E., Larsen, T., Young, H. A., Reed, D. S., Geisbert, J. B., Scott, D. P., Kagan, R, Jahrling, P. B., and Davis, K. J. (2003) Pathogenesis of Ebola hemorrhagic fever in cynomolgus macaques: evidence that dendritic cells are early and sustained targets of infection. Am J Pathol 163, 2347-2370
Sanchez, A., Lukwiya, M., Bausch, D., Mahanty, S., Sanchez, A. J., Wagoner, K. D., and Rollin, P. E. (2004) Analysis of human peripheral blood samples from fatal and nonfatal cases of Ebola (Sudan) hemorrhagic fever: cellular responses, virus load, and nitric oxide levels. J Virol 78, 10370-10377
Baize, S., Leroy, E. M, Georges-Courbot, M. C, Capron, M., Lansoud-Soukate, J., Debre, P., Fisher-Hoch, S. P., McCormick, J. B., and Georges, A. J. (1999) Defective humoral responses and extensive intravascular apoptosis are associated with fatal outcome in Ebola virus-infected patients. Nat Med 5,423-426
Bukreyev, A., Volchkov, V. E., Blinov, V. M., and Netesov, S. V. (1993) The GP-protein of
Marburg virus contains the region similar to the ‘immunosuppressive domain’ of oncogenic retrovirus P15E proteins. FEBS Lett 323, 183-187
Volchkov, V. E., Blinov, V. M., and Netesov, S. V. (1992) The envelope glycoprotein of Ebola virus contains an immunosuppressive-like domain similar to oncogenic retroviruses. FEBS Lett 305, 181-184
Leroy, E. M., Baize, S., Volchkov, V. E., Fisher-Hoch, S. P., Georges-Courbot, M. C, Lansoud-Soukate, J., Capron, M., Debre, P., McCormick, J. B., and Georges, A. J. (2000) Human asymptomatic Ebola infection and strong inflammatory response. Lancet 355, 2210-2215
Villinger, F., Rollin, P. E., Brar, S. S., Chikkala, N. F., Winter, J., Sundstrom, J. B., Zaki, S. R., Swanepoel, R., Ansari, A. A., and Peters, C. J. (1999) Markedly elevated levels of interferon (IFN)-gamma, IFN-alpha, interleukin (IL)-2, IL-10, and tumor necrosis factor-alpha associated with fatal Ebola virus infection. J Infect Dis 179 Suppl 1, S188-191
Yang, Z. Y., Duckers, H. J., Sullivan, N. J., Sanchez, A., Nabel, E. G., and Nabel, G. J. (2000) Identification of the Ebola virus glycoprotein as the main viral determinant of vascular cell cytotoxicity and injury. Nat Med 6, 886-889
Volchkov, V. E., Volchkova, V. A., Muhlberger, E., Kolesnikova, L. V., Weik, M., Dolnik, O., and Klenk, H. D. (2001) Recovery of infectious Ebola virus from complementary DNA: RNA editing of the GP gene and viral cytotoxicity. Science 291, 1965-1969
Feldmann, H., Volchkov, V. E, Volchkova, V. A., Stroher, U., and Klenk, H. D. (2001) Biosynthesis and role of filoviral glycoproteins. J Gen Virol 82, 2839-2848
Denner, J., Norley, S., and Kurth, R. (1994) The immunosuppressive peptide of HIV-1: functional domains and immune response in AIDS patients. Aids 8, 1063-1072
Haraguchi, S., Good, R. A., and Day, N. K. (1995) Immunosuppressive retroviral peptides: cAMP and cytokine patterns. Immunol Today 16, 595-603
Cianciolo, G. J., Copeland, T. D., Oroszlan, S., and Snyderman, R. (1985) Inhibition of lymphocyte proliferation by a synthetic peptide homologous to retroviral envelope proteins. Science 230, 453-455
Haraguchi, S., Good, R. A., James-Yarish, M., Cianciolo, G. J., and Day, N. K. (1995) Induction of intracellular cAMP by a synthetic retroviral envelope peptide: a possible mechanism of immunopathogenesis in retroviral infections. Proc Natl Acad Sci USA 92, 5568-5571
Haraguchi, S., Good, R. A., James-Yarish, M L, Cianciolo, G. J., and Day, N. K. (1995) Differential modulation of Th1- and Th2-related cytokine mRNA expression by a synthetic peptide homologous to a conserved domain within retroviral envelope protein. Proc Natl Acad Sci USA 92, 3611-3615
Gottlieb, R. A., Kleinerman, E. S., O'Brian, C. A., Tsujimoto, S., Cianciolo, G. J., and Lennarz, W. J. (1990) Inhibition of protein kinase C by a peptide conjugate homologous to a domain of the retroviral protein p15E. J Immunol 145, 2566-2570
Kadota, J., Cianciolo, G. J., and Snyderman, R. (1991) A synthetic peptide homologous to retroviral transmembrane envelope proteins depresses protein kinase C mediated lymphocyte proliferation and directly inactivated protein kinase C: a potential mechanism for immunosuppression. Microbiol Immunol 35, 443-459
Kelley, L. A., MacCallum, R. M., and Steinberg, M. J. (2000) Enhanced genome annotation using structural profiles in the program 3D-PSSM. J Mol Biol 299, 499-520
Thompson, C. B., Lindsten, T., Ledbetter, J. A., Kunkel, S. L., Young, H. A., Emerson, S. G., Leiden, J. M L, and June, C. H. (1989) CD28 activation pathway regulates the production of multiple T-cell-derived lymphokines/cytokines. Proc Natl Acad Sci USA 86, 1333-1337
Sancho, J., Ledbetter, J. A., Choi, M. S., Kanner, S. B., Deans, J. P., and Terhorst, C. (1992) CD3-zeta surface expression is required for CD4-p561ck-mediated upregulation of T cell antigen receptor-CD3 signaling in T cells. J Biol Chem 267, 7871-7879
Jacobsen, C. N., Aasted, B., Broe, M. K., and Petersen, J. L. (1993) Reactivities of 20 anti-human monoclonal antibodies with leucocytes from ten different animal species. Vet Immunol Immunopathol 39, 461-466
Sopper, S., Stahl-Hennig, C, Demuth, M., Johnston, I. C, Dorries, R., and ter Meulen, V. (1997) Lymphocyte subsets and expression of differentiation markers in blood and lymphoid organs of rhesus monkeys. Cytometry 29, 351-362
Waldmann, T. A. (1991) The interleukin-2 receptor. J Biol Chem 266, 2681-2684
Hara, T., Jung, L. K., Bjorndahl, J. M., and Fu, S. M. (1986) Human T cell activation. III. Rapid induction of a phosphorylated 28 kD/32 kD disulfide-linked early activation antigen (EA 1) by 12-o-tetradecanoyl phorbol-13-acetate, mitogens, and antigens. J Exp Med 164, 1988-2005
Nicoletti, I., Migliorati, G., Pagiiacci, M. C, Grignani, R, and Riccardi, C. (1991) A rapid and simple method for measuring thymocyte apoptosis by propidium iodide staining and flow cytometry. J Immunol Methods 139, 271-279
Geisbert, T. W., Hensley, L. E., Gibb, T. R., Steele, K. E., Jaax, N. K., and Jahrling, P. B. (2000) Apoptosis induced in vitro and in vivo during infection by Ebola and Marburg viruses. Lab Invest 80, 171-186
Hensley, L. E., Young, H. A., Jahrling, P. B., and Geisbert, T. W. (2002) Proinflammatory response during Ebola virus infection of primate models: possible involvement of the tumor necrosis factor receptor superfamily. Immunol Lett 80,169-179
Baize, S., Leroy, E. M, Georges, A. J., Georges-Courbot, M. C, Capron, M, Bedjabaga, I.,
Lansoud-Soukate, J., and Mavoungou, E. (2002) Inflammatory responses in Ebola virus-infected patients. Clin Exp Immunol 128, 163-168
Basler, C. E, Mikulasova, A., Martinez-Sobrido, L., Paragas, J., Muhlberger, E., Bray, M L, Klenk, H. D., Palese, P., and Garcia-Sastre, A. (2003) The Ebola virus VP35 protein inhibits activation of interferon regulatory factor J Virol 77, 7945-7956
Reid, S. L., L. W. Hartman, A. L. Martinez, O. Shaw, M. L. Carbonnelle, C. Volchkov, V. E. nichol, S. T. Basler, C. F. (2006) Ebola virus VP24 binds Karyopherin al and blocks STAT1 nuclear accumulation. J Virol 80, 1-12
Gupta, M., Mahanty, S., Ahmed, R, and Rollin, P. E. (2001) Monocyte-derived human macrophages and peripheral blood mononuclear cells infected with ebola virus secrete MIP-1 alpha and TNF-alpha and inhibit poly-IC-induced IFN-alpha in vitro. Virology 284, 20-25
Harcourt, B. H., Sanchez, A., and Offerrnann, M. K. (1998) Ebola virus inhibits induction of genes by double-stranded RNA in endothelial cells. Virology 252, 179-188
Mahanty, S., Hutchinson, K., Agarwal, S., McRae, M., Rollin, P. E., and Pulendran, B. (2003) Cutting edge: impairment of dendritic cells and adaptive immunity by Ebola and Lassa viruses. J Immunol 170, 2797-2801
Bosio, C. M., Aman, M. J., Grogan, C, Hogan, R., Ruthel, G., Negley, D., Mohamadzadeh, M., Bavari, S., and Schmaljohn, A. (2003) Ebola and Marburg viruses replicate in monocyte-derived dendritic cells without inducing the production of cytokines and full maturation. J Infect Dis 188, 1630-1638
D'Andrea, A., Rengaraju, M., Valiante, N. M., Chehimi, J., Kubin, M., Aste, M., Chan, S. H., Kobayashi, M., Young, D., Nickbarg, E., and et al. (1992) Production of natural killer cell stimulatory factor (interleukin 12) by peripheral blood mononuclear cells. J Exp Med 176, 1387-1398
Wolf, S. R, Temple, P. A., Kobayashi, M, Young, D., Dicig, M., Lowe, L., Dzialo, R., Fitz, L., Ferenz, C, Hewick, R. M., and et al. (1991) Cloning of cDNA for natural killer cell stimulatory factor, a heterodimeric cytokine with multiple biologic effects on T and natural killer cells. J Immunol 146, 3074-3081
de Waal Malefyt, R., Yssel, H., and de Vries, J. E. (1993) Direct effects of IL-10 on subsets of human CD4+ T cell clones and resting T cells. Specific inhibition of IL-2 production and proliferation. J Immunol 150, 4754-4765
Ding, L., Linsley, P. S., Huang, L. Y., Germain, R. R, and Shevach, E. M. (1993) IL-10 inhibits macrophage costimulatory activity by selectively inhibiting the up-regulation of B7 expression. J Immunol 151, 1224-1234
Hartman, A. L, Towner, J. S., and Nichol, S. T. (2004) A C-terminal basic amino acid motif of Zaire ebolavirus VP35 is essential for type I interferon antagonism and displays high identity with the RNA-binding domain of another interferon antagonist, the NS1 protein of influenza A virus. Virology 328, 177-184

1-21. (canceled)

22. A method for modulating or suppressing an immune response of a subject, the method comprising administering to the subject in an effective amount so as to suppress the immune response in the subject:
a) an isolated peptide comprising the consecutive amino acid sequence of any one of SEQ ID NOs: 1, or 108, wherein the total length of the peptide is less than 26 amino acids, and wherein the peptide has immunosuppressive activity, wherein the immunosuppressive activity is determined in vitro by increased production of an anti-inflammatory cytokine, decreased production of a pro-inflammatory cytokine, or a combination thereof by human peripheral blood mononuclear cells (PBMCs); or
b) an isolated peptide consisting of the consecutive amino acid sequence of any one of SEQ ID NOs: 1, or 108, wherein the peptide has immunosuppressive activity, wherein the immunosuppressive activity is determined in vitro by increased production of an anti-inflammatory cytokine, decreased production of a pro-inflammatory cytokine, or a combination thereof by human peripheral blood mononuclear cells (PBMCs).

23. The method of claim 22, wherein the subject suffers from an autoimmune disease.

24. The method of claim 23, wherein the autoimmune disease is one or more of diabetes mellitus, rheumatoid arthritis, multiple sclerosis, systemic lupus erythematosis, myasthenia gravis, scleroderma, inflammatory bowel disease, Crohn's disease, ulcerative colitis, Hashimoto's thyroiditis, Graves' disease, Sjogren's syndrome, polyendocrine failure, vitiligo, peripheral neuropathy, rejection of transplantation, graft-versus-host disease, autoimmune polyglandular syndrome type I, acute glomerulonephritis, Addison's disease, adult-onset idiopathic hypoparathyroidism (AOIH), alopecia totalis, amyotrophic lateral sclerosis, ankylosing spondylitis, autoimmune aplastic anemia, autoimmune hemolytic anemia, Behcet's disease, Celiac disease, chronic active hepatitis, CREST syndrome, dermatomyositis, dilated cardiomyopathy, eosinophilia-myalgia syndrome, epidermolisis bullosa acquisita (EBA), giant cell arteritis, Goodpasture's syndrome, Guillain-Barre syndrome, hemochromatosis, HenochSchonlein purpura, idiopathic IgA nephropathy, juvenile rheumatoid arthritis, Lambert-Eaton syndrome, linear IgA dermatosis, myocarditis, narcolepsy, necrotizing vasculitis, neonatal lupus syndrome (NLE), nephrotic syndrome, pemphigoid, pemphigus, polymyositis, primary sclerosing cholangitis, psoriasis, rapidly-progressive glomerulonephritis (RPGN), Reiter's syndrome, stiff-man syndrome, thyroiditis, inflammatory bowel disease or any combination thereof.

25. A method for identifying an agent that modulates an immunosuppressive bioactivity of an immunosuppressive peptide, the method comprising:
a) contacting a cell exposed to the immunosuppressive peptide with an agent,
b) determining whether the cell exhibits an inhibited or an increased immune response, wherein exhibition of increased immune response is indicative of an agent that modulates the immunosuppressive effect of the peptide;
wherein the immunosuppressive peptide is an isolated peptide comprising the consecutive amino acid sequence of any one of SEQ ID NOs: 1, or 108, wherein the total length of the peptide is less than 26 amino acids, and wherein the peptide has immunosuppressive activity, wherein the immunosuppressive activity is determined in vitro by increased production of an anti-inflammatory cytokine, decreased production of a pro-inflammatory cytokine, or a combination thereof by human peripheral blood mononuclear cells (PBMCs); or wherein the immunosuppressive peptide is an isolated peptide consisting of the consecutive amino acid sequence of any one of SEQ ID NOs: 1, or 108, wherein the peptide has immunosuppressive activity, wherein the immunosuppressive activity is determined in vitro by increased production of an anti-inflammatory cytokine, decreased production of a pro-inflammatory cytokine, or a combination thereof by human peripheral blood mononuclear cells (PBMCs).

26. The method of claim 25, wherein the cell is a CD4+ cell, CD8+ cell, a cell in a population of cells as comprised in PBMCs, or a mixture thereof.

27. The method of claim 25, wherein the determining step comprises comparing cell proliferation or levels of cytokines produced by the cell in the presence of the agent with the levels determined in the absence of the agent.

28. A method for treating disorders associated with hyperproliferation of lymphocytes comprising administering to a subject an effective amount of:
a) an isolated peptide comprising the consecutive amino acid sequence of any one of SEQ ID NOs: 1, or 108, wherein the total length of the peptide is less than 26 amino acids, and wherein the peptide has immunosuppressive activity, wherein the immunosuppressive activity is determined in vitro by increased production of an anti-inflammatory cytokine, decreased production of a pro-inflammatory cytokine, or a combination thereof by human peripheral blood mononuclear cells (PBMCs); or
b) an isolated peptide consisting of the consecutive amino acid sequence of any one of SEQ ID NOs: 1, or 108, wherein the peptide has immunosuppressive activity, wherein the immunosuppressive activity is determined in vitro by increased production of an anti-inflammatory cytokine, decreased production of a pro-inflammatory cytokine, or a combination thereof by human peripheral blood mononuclear cells (PBMCs).

29-32. (canceled)


SEQUENCE LISTING (381 sequences)

SEQ ID NO: 1 - protein, 17 aa, Zaire ebolavirus
ILNRKAIDFLLQRWGGT

SEQ ID NO: 2 - protein, 17 aa, Lake Victoria marburgvirus
LINRHAIDFLLTRWGGT

SEQ ID NO: 3 - protein, 17 aa, Sudan ebolavirus
ILNRKAIDFLLRRWGGT

SEQ ID NO: 4 - protein, 17 aa, Reston ebolavirus
LLNRKAIDFLLQRWGGT

SEQ ID NO: 5 - protein, 17 aa, Ivory Coast ebolavirus
ILNRKAIDFLLQRWGGT

SEQ ID NO: 6 - protein, 17 aa, Artificial Sequence
  Description of Artificial Sequence: Synthetic peptide
LQNRRGLDLLFLKEGGL

SEQ ID NO: 7 - protein, 17 aa, Artificial Sequence
  Description of Artificial Sequence: Synthetic peptide
LQNRRGLDLLFLKEGGL

SEQ ID NO: 8 - protein, 16 aa, Artificial Sequence
  Description of Artificial Sequence: Synthetic peptide
LQNRRGDLLFLKEGGL

SEQ ID NO: 9 - protein, 17 aa, Artificial Sequence
  Description of Artificial Sequence: Synthetic peptide
LQNRRGLDLLFLREGGL

SEQ ID NO: 10 - protein, 17 aa, Artificial Sequence
  Description of Artificial Sequence: Synthetic peptide
LQNRRGLDLLFLKEGGL

SEQ ID NO: 11 - protein, 17 aa, Artificial Sequence
  Description of Artificial Sequence: Synthetic peptide
  MOD_RES (5)..(5): Variable amino acid
LQNRXGLDLLFLSQGEL

SEQ ID NO: 12 - protein, 17 aa, Artificial Sequence
  Description of Artificial Sequence: Synthetic peptide
LQNCRGLDLLFLSQGGL

SEQ ID NO: 13 - protein, 17 aa, Artificial Sequence
  Description of Artificial Sequence: Synthetic peptide
LQNRRGLDLLFLSQGGL

SEQ ID NO: 14 - protein, 17 aa, Artificial Sequence
  Description of Artificial Sequence: Synthetic peptide
  MOD_RES (12)..(12): Variable amino acid
AQNRRGLDLLFXEQGGL

SEQ ID NO: 15 - protein, 17 aa, Artificial Sequence
  Description of Artificial Sequence: Synthetic peptide
LQNRRGLDLLTAEQGGI

SEQ ID NO: 16 - protein, 17 aa, Artificial Sequence
  Description of Artificial Sequence: Synthetic peptide
  MOD_RES (17)..(17): Variable amino acid
LQNRRGLDLLTAEQGGX

SEQ ID NO: 17 - protein, 17 aa, Artificial Sequence
  Description of Artificial Sequence: Synthetic peptide
LQNRRGLDMLTAAQGGI

SEQ ID NO: 18 - protein, 17 aa, Artificial Sequence
  Description of Artificial Sequence: Synthetic peptide
LQNYQELDELTAAQRET

SEQ ID NO: 19 - protein, 17 aa, Artificial Sequence
  Description of Artificial Sequence: Synthetic peptide
LQNCQGLDMLMAAQGGI

SEQ ID NO: 20 - protein, 17 aa, Artificial Sequence
  Description of Artificial Sequence: Synthetic peptide
  MOD_RES (5)..(5): Variable amino acid
LQNRXGLDLLTAEKGGL

SEQ ID NO: 21 - protein, 17 aa, Artificial Sequence
  Description of Artificial Sequence: Synthetic peptide
LQNCRGLDLLTAEKGGP

SEQ ID NO: 22 - protein, 17 aa, Artificial Sequence
  Description of Artificial Sequence: Synthetic peptide
LQNRRGLDLLTAEKGGL

SEQ ID NO: 23 - protein, 17 aa, Artificial Sequence
  Description of Artificial Sequence: Synthetic peptide
LQNHRGLNLLTAEKGRL

SEQ ID NO: 24 - protein, 17 aa, Artificial Sequence
  Description of Artificial Sequence: Synthetic peptide
LQNRRGLNMLTAEKRGL

SEQ ID NO: 25 - protein, 17 aa, Artificial Sequence
  Description of Artificial Sequence: Synthetic peptide
LQNRKGLDLLTAEKGSL

SEQ ID NO: 26 - protein, 17 aa, Artificial Sequence
  Description of Artificial Sequence: Synthetic peptide
LQNRKGLNLLTAEKGGL

SEQ ID NO: 27 - protein, 17 aa, Artificial Sequence
  Description of Artificial Sequence: Synthetic peptide
  MOD_RES (8)..(8): Variable amino acid
LQNRRGPXLLTAEKGGL

SEQ ID NO: 28 - protein, 17 aa, Artificial Sequence
  Description of Artificial Sequence: Synthetic peptide
FQNCRGLDLLTAEKGGL

SEQ ID NO: 29 - protein, 17 aa, Artificial Sequence
  Description of Artificial Sequence: Synthetic peptide
  MOD_RES (5)..(5): Variable amino acid
LQNCXGLDLLTVEEGGF

SEQ ID NO: 30 - protein, 16 aa, Artificial Sequence
  Description of Artificial Sequence: Synthetic peptide
LQNRALDLLIAKRGGT

SEQ ID NO: 31 - protein, 17 aa, Artificial Sequence
  Description of Artificial Sequence: Synthetic peptide
LQNRRALDLLTAKRGGT

SEQ ID NO: 32 - protein, 17 aa, Artificial Sequence
  Description of Artificial Sequence: Synthetic peptide
LQNRRALDLLTAERGGT

SEQ ID NO: 33 - protein, 17 aa, Artificial Sequence
  Description of Artificial Sequence: Synthetic peptide
LQNQRALNLLTAEQGGT

SEQ ID NO: 34 - protein, 17 aa, Artificial Sequence
  Description of Artificial Sequence: Synthetic peptide
LQNRRALDLLTAEQGGT

SEQ ID NO: 35 - protein, 17 aa, Artificial Sequence
  Description of Artificial Sequence: Synthetic peptide
LQNQRALDLLAAEKGSP

SEQ ID NO: 36 - protein, 17 aa, Artificial Sequence
  Description of Artificial Sequence: Synthetic peptide
AQNRRALDLLTAEKGGT

SEQ ID NO: 37 - protein, 17 aa, Artificial Sequence
  Description of Artificial Sequence: Synthetic peptide
AQNRQALDLLMAEKGRT

SEQ ID NO: 38 - protein, 17 aa, Artificial Sequence
  Description of Artificial Sequence: Synthetic peptide
LNNRLAVDYLLAQVGEV

SEQ ID NO: 39 - protein, 17 aa, Artificial Sequence
  Description of Artificial Sequence: Synthetic peptide
  MOD_RES (8)..(9): Variable amino acid
  MOD_RES (16)..(16): Variable amino acid
LNNRLALXXLLTEQSXA

SEQ ID NO: 40 - protein, 17 aa, Artificial Sequence
  Description of Artificial Sequence: Synthetic peptide
  MOD_RES (14)..(14): Variable amino acid
LNNRLMLDCLLAVXGRI

SEQ ID NO: 41 - protein, 16 aa, Artificial Sequence
  Description of Artificial Sequence: Synthetic peptide
  MOD_RES (7)..(7): Variable amino acid
LQNQLTXEVLPAEGGT

SEQ ID NO: 42 - protein, 17 aa, Artificial Sequence
  Description of Artificial Sequence: Synthetic peptide
LQNQHALDVLTTKAGGT

SEQ ID NO: 43 - protein, 17 aa, Artificial Sequence
  Description of Artificial Sequence: Synthetic peptide
AQNRQALDVITAEVGGT

SEQ ID NO: 44 - protein, 17 aa, Artificial Sequence
  Description of Artificial Sequence: Synthetic peptide
  MOD_RES (15)..(15): Variable amino acid
AQNRQALDVLTTEVXGT

SEQ ID NO: 45 - protein, 17 aa, Artificial Sequence
  Description of Artificial Sequence: Synthetic peptide
MQNRQALDILMAKVGGT

SEQ ID NO: 46 - protein, 17 aa, Artificial Sequence
  Description of Artificial Sequence: Synthetic peptide
WENRLQLDIILAEKGVV

SEQ ID NO: 47 - protein, 17 aa, Artificial Sequence
  Description of Artificial Sequence: Synthetic peptide
WENKIALNIILAVNGSV

SEQ ID NO: 48 - protein, 17 aa, Artificial Sequence
  Description of Artificial Sequence: Synthetic peptide
  MOD_RES (1)..(1): Variable amino acid
XENRMAIGNILAEKGRV

SEQ ID NO: 49 - protein, 17 aa, Artificial Sequence
  Description of Artificial Sequence: Synthetic peptide
WENRIALDMTLAKEGGV

SEQ ID NO: 50 - protein, 17 aa, Artificial Sequence
  Description of Artificial Sequence: Synthetic peptide
WENKIALDMIPAKEGGD

SEQ ID NO: 51 - protein, 17 aa, Artificial Sequence
  Description of Artificial Sequence: Synthetic peptide
LQNRMALDILTAAPGGT

SEQ ID NO: 52 - protein, 17 aa, Artificial Sequence
  Description of Artificial Sequence: Synthetic peptide
LQNHMALDILTVAQGGT

SEQ ID NO: 53 - protein, 17 aa, Artificial Sequence
  Description of Artificial Sequence: Synthetic peptide
LQNCMALDTLSAAQSET

SEQ ID NO: 54 - protein, 16 aa, Artificial Sequence
  Description of Artificial Sequence: Synthetic peptide
LQNRMSLDIVTTAQGG

SEQ ID NO: 55 - protein, 17 aa, Artificial Sequence
  Description of Artificial Sequence: Synthetic peptide
LQNWMALDIVTADQGGT

SEQ ID NO: 56 - protein, 17 aa, Artificial Sequence
  Description of Artificial Sequence: Synthetic peptide
LQNQMALDILTAPQGGT

SEQ ID NO: 57 - protein, 17 aa, Artificial Sequence
  Description of Artificial Sequence: Synthetic peptide
LQNCMALDIFMAAQEGT

SEQ ID NO: 58 - protein, 17 aa, Artificial Sequence
  Description of Artificial Sequence: Synthetic peptide
LQNHMALDTLIAAQGGT

SEQ ID NO: 59 - protein, 17 aa, Artificial Sequence
  Description of Artificial Sequence: Synthetic peptide
LYNHMALDILIAAQGGT

SEQ ID NO: 60 - protein, 17 aa, Artificial Sequence
  Description of Artificial Sequence: Synthetic peptide
  MOD_RES (2)..(2): Variable amino acid
LXNRMALDILTAAQGGT

SEQ ID NO: 61 - protein, 17 aa, Artificial Sequence
  Description of Artificial Sequence: Synthetic peptide
LQNRMALDILTAAEGGT

SEQ ID NO: 62 - protein, 17 aa, Artificial Sequence
  Description of Artificial Sequence: Synthetic peptide
LQNQMALDMLTATQGGV

SEQ ID NO: 63 - protein, 17 aa, Artificial Sequence
  Description of Artificial Sequence: Synthetic peptide
LQNHVAPDMLTAAQGGV

SEQ ID NO: 64 - protein, 17 aa, Artificial Sequence
  Description of Artificial Sequence: Synthetic peptide
LQNQMALHILTAAQGRV

SEQ ID NO: 65 - protein, 17 aa, Artificial Sequence
  Description of Artificial Sequence: Synthetic peptide
LQNRAAIDFLLLAHGHG

SEQ ID NO: 66 - protein, 17 aa, Artificial Sequence
  Description of Artificial Sequence: Synthetic peptide
  MOD_RES (9)..(9): Variable amino acid
YQNRLPLDXLLAEESGV

SEQ ID NO: 67 - protein, 17 aa, Artificial Sequence
  Description of Artificial Sequence: Synthetic peptide
YQNRLALDYLLAEEGGV

SEQ ID NO: 68 - protein, 17 aa, Artificial Sequence
  Description of Artificial Sequence: Synthetic peptide
YQNRLALDYLLAQEEGV

SEQ ID NO: 69 - protein, 17 aa, Artificial Sequence
  Description of Artificial Sequence: Synthetic peptide
YQNRLALDYLLAQEGGV

SEQ ID NO: 70 - protein, 17 aa, Artificial Sequence
  Description of Artificial Sequence: Synthetic peptide
YQNRLGLDYLLAQEGGI

SEQ ID NO: 71 - protein, 17 aa, Artificial Sequence
  Description of Artificial Sequence: Synthetic peptide
  MOD_RES (2)..(2): Variable amino acid
YXNRLALDYHLASEGRV

SEQ ID NO: 72 - protein, 17 aa, Artificial Sequence
  Description of Artificial Sequence: Synthetic peptide
YQNRLALDYLLALEGGV

SEQ ID NO: 73 - protein, 17 aa, Artificial Sequence
  Description of Artificial Sequence: Synthetic peptide
YQNRLALDYLLASEGGV

SEQ ID NO: 74 - protein, 17 aa, Artificial Sequence
  Description of Artificial Sequence: Synthetic peptide
YQNRLALDYLLAAEGGV

SEQ ID NO: 75 - protein, 16 aa, Artificial Sequence
  Description of Artificial Sequence: Synthetic peptide
YQNRLALNYLLAAEGG

SEQ ID NO: 76 - protein, 17 aa, Artificial Sequence
  Description of Artificial Sequence: Synthetic peptide
YQNRLALDYLIAAEGGV

SEQ ID NO: 77 - protein, 17 aa, Artificial Sequence
  Description of Artificial Sequence: Synthetic peptide
LINRHAIDFLLTRWGGT

SEQ ID NO: 78 - protein, 17 aa, Artificial Sequence
  Description of Artificial Sequence: Synthetic peptide
LAVERYLKDQQLLGIWG

SEQ ID NO: 79 - protein, 17 aa, Artificial Sequence
  Description of Artificial Sequence: Synthetic peptide
LELGQDVANLKTRNSTK

SEQ ID NO: 80 - protein, 17 aa, Artificial Sequence
  Description of Artificial Sequence: Synthetic peptide
  MOD_RES (8)..(8): Variable amino acid
LWLGEQVXSLQLQRQLR

SEQ ID NO: 81 - protein, 17 aa, Artificial Sequence
  Description of Artificial Sequence: Synthetic peptide
  MOD_RES (2)..(2): Variable amino acid
IXMEDRTINLKHQLEVQ

SEQ ID NO: 82 - protein, 17 aa, Artificial Sequence
  Description of Artificial Sequence: Synthetic peptide
  MOD_RES (13)..(13): Variable amino acid
IWLGDRMMNLEHXMQLQ

SEQ ID NO: 83 - protein, 17 aa, Artificial Sequence
  Description of Artificial Sequence: Synthetic peptide
IWMGDRLMSLEHRFQLQ

SEQ ID NO: 84 - protein, 17 aa, Artificial Sequence
  Description of Artificial Sequence: Synthetic peptide
DLAEEQIGVLHQMAQLG

SEQ ID NO: 85 - protein, 17 aa, Moloney-Murine Leukemia Virus
DLAEEQIGVLHQMAQLG

SEQ ID NO: 86 - protein, 17 aa, Artificial Sequence
  Description of Artificial Sequence: Synthetic peptide
LQNRRGLDLLFLKEGGL

SEQ ID NO: 87 - protein, 8 aa, Artificial Sequence
  Description of Artificial Sequence: Synthetic peptide
KSPWFTTL

SEQ ID NO: 88 - RNA, 54 nt, Artificial Sequence
  Description of Artificial Sequence: Synthetic oligonucleotide
auuuuaaaucgcaaagcuauugauuuuuuauuacaacgcuggggcggcacuuga

SEQ ID NO: 89 - RNA, 54 nt, Artificial Sequence
  Description of Artificial Sequence: Synthetic oligonucleotide
uuacaaaaucgccgcggcuuagauuuauuauuuuuaaaagaaggcggcuuauga

SEQ ID NO: 90 - RNA, 51 nt, Artificial Sequence
  Description of Artificial Sequence: Synthetic oligonucleotide
uuacaaaaucgccgcggcgauuuauuauuuuuaaaagaaggcggcuuauga

SEQ ID NO: 91 - RNA, 54 nt, Artificial Sequence
  Description of Artificial Sequence: Synthetic oligonucleotide
uuacaaaaucgccgcggcuuagauuuauuauuuuuacgcgaaggcggcuuauga

SEQ ID NO: 92 - RNA, 54 nt, Artificial Sequence; identical to SEQ ID NO: 89
  Description of Artificial Sequence: Synthetic oligonucleotide

SEQ ID NO: 93 - RNA, 54 nt, Artificial Sequence
  Description of Artificial Sequence: Synthetic oligonucleotide
  modified_base (13)..(15): a, c, g, or u
uuacaaaaucgcnnnggcuuagauuuauuauuuuuaucucaaggcgaauuauga

SEQ ID NO: 94 - RNA, 54 nt, Artificial Sequence
  Description of Artificial Sequence: Synthetic oligonucleotide
uuacaaaauugucgcggcuuagauuuauuauuuuuaucucaaggcggcuuauga

SEQ ID NO: 95 - RNA, 54 nt, Artificial Sequence
  Description of Artificial Sequence: Synthetic oligonucleotide
uuacaaaaucgccgcggcuuagauuuauuauuuuuaucucaaggcggcuuauga

SEQ ID NO: 96 - RNA, 54 nt, Artificial Sequence
  Description of Artificial Sequence: Synthetic oligonucleotide
uuacaaaaucgccgcggcuuagauuuauuaacugcugaacaaggcggcauuuga

SEQ ID NO: 97 - RNA, 54 nt, Artificial Sequence
  Description of Artificial Sequence: Synthetic oligonucleotide
uuacaaaaucgccgcggcuuagauauguuaacugcugcucaaggcggcauuuga

SEQ ID NO: 98 - RNA, 54 nt, Artificial Sequence
  Description of Artificial Sequence: Synthetic oligonucleotide
uuacaaaauuaucaagaauuagaugaauuaacugcugcucaacgcgaaacuuga

SEQ ID NO: 99 - RNA, 54 nt, Artificial Sequence
  Description of Artificial Sequence: Synthetic oligonucleotide
uuacaaaauugucgcggcuuagauuuauuaacugcugaaaaaggcggcccuuga

SEQ ID NO: 100 - RNA, 54 nt, Artificial Sequence
  Description of Artificial Sequence: Synthetic oligonucleotide
uuacaaaaucgccgcggcuuaaauauguuaacugcugaaaaacgcggcuuauga

SEQ ID NO: 101 - RNA, 54 nt, Artificial Sequence
  Description of Artificial Sequence: Synthetic oligonucleotide
uuucaaaauugucgcggcuuagauuuauuaacugcugaaaaaggcggcuuauga

SEQ ID NO: 102 - RNA, 54 nt, Artificial Sequence
  Description of Artificial Sequence: Synthetic oligonucleotide
uuacaaaaucgccgcgcuuuagauuuauuaggcgcuaaacgcggcggcacuuga

SEQ ID NO: 103 - RNA, 54 nt, Artificial Sequence
  Description of Artificial Sequence: Synthetic oligonucleotide
uuacaaaaucaacgcgcuuuaaauuuauuacguacugcugaaggcggcacuuga

SEQ ID NO: 104 - RNA, 54 nt, Artificial Sequence
  Description of Artificial Sequence: Synthetic oligonucleotide
uuacaaaaucaacgcgcuuuagauuuauuagcugcugaaaaaggcucuccuuga

SEQ ID NO: 105 - RNA, 54 nt, Artificial Sequence
  Description of Artificial Sequence: Synthetic oligonucleotide
gcucaaaaucgccaagcuuuagauuuauuaauggcugaaaaaggccgcacuuga

SEQ ID NO: 106 - RNA, 54 nt, Artificial Sequence
  Description of Artificial Sequence: Synthetic oligonucleotide
uuaaauaaucgcuuagcuguugauuauuuauuagcucaaguuggcgaaguuuga

SEQ ID NO: 107 - RNA, 54 nt, Artificial Sequence
  Description of Artificial Sequence: Synthetic oligonucleotide
uuacaaaaucaacaugcuuuagauguuuuaacuacuaaagcuggcggcacuuga

SEQ ID NO: 108 - protein, 18 aa, Artificial Sequence
  Description of Artificial Sequence: Synthetic oligonucleotide
ILNRKAIDFLLQRWGGTC

SEQ ID NO: 109 - protein, 18 aa, Zaire ebolavirus
ILNRKAIDFLLQRWGGTC

SEQ ID NO: 110 - protein, 18 aa, Zaire ebolavirus
ILNRKAIDFLLQRWGGTC

SEQ ID NO: 111 - protein, 18 aa, Zaire ebolavirus
ILNRKAIDFLLQRWGGTC

SEQ ID NO: 112 - protein, 18 aa, Zaire ebolavirus
ILNRKAIDFLLQRWGGTC

SEQ ID NO: 113 - protein, 18 aa, Zaire ebolavirus
LLNRKAIDFLLQRWGGTC

SEQ ID NO: 114 - protein, 18 aa, Zaire ebolavirus
LLNRKAIDFLLQRWGGTC

SEQ ID NO: 115 - protein, 18 aa, Zaire ebolavirus
ILNRKAIDFLLRRWGGTC

SEQ ID NO: 116 - protein, 18 aa, Zaire ebolavirus
ILNRKAIDFLLRRWGGTC

SEQ ID NO: 117 - protein, 18 aa, Marburg virus
LINRHAIDFLLTRWGGTC

SEQ ID NO: 118 - protein, 18 aa, Marburg virus
LINRHAIDFLLTRWGGTC

SEQ ID NO: 119 - protein, 18 aa, Marburg virus
LINRHAIDFLLTRWGGTC

SEQ ID NO: 120 - protein, 18 aa, Marburg virus
LINRHAIDFLLTRWGGTC

SEQ ID NO: 121 - protein, 18 aa, Marburg virus
LINRHAIDFLLARWGGTC

SEQ ID NO: 122 - protein, 18 aa, Moloney Murine Leukemia Virus
LQNRRGLDLLFLKEGGLC

SEQ ID NO: 123 - protein, 18 aa, Human T-cell leukemia virus
AQNRRGLDLLFWEQGGLC

SEQ ID NO: 124 - protein, 18 aa, Human Endogenous Retrovirus
YQNRLALDYLLAQEEGVC

SEQ ID NO: 125 - protein, 18 aa, Human Endogenous Retrovirus
MNNRLALDYLLAEQGGVC

SEQ ID NO: 126 - protein, 18 aa, Friend murine leukemia virus
LQNRRGLDLLFLKEGGLC

SEQ ID NO: 127 - protein, 18 aa, Avian Sarcoma virus
LQNRAAIDFLLLAHGHGC

SEQ ID NO: 128 - protein, 18 aa, Zebrafish endogenous retrovirus
IQNRLALDMLLSERGGVC

SEQ ID NO: 129 - protein, 18 aa, Avian reticuloendotheliosis virus
LQNRRGLDLLTAEQGGIC

SEQ ID NO: 130 - protein, 18 aa, Avian spleen necrosis virus
LQNRRGLDLLTAEQGGIC

SEQ ID NO: 131 - protein, 18 aa, Avian sarcoma virus
LQNRAAIDFLLLAHGHGC

SEQ ID NO: 132 - protein, 18 aa, Baboon endogenous virus
LQNRRGLDLLTAEQGGIC

SEQ ID NO: 133 - protein, 18 aa, Feline endogenous virus ECE1
LQNRRGLDLLFLQEGGLC

SEQ ID NO: 134 - protein, 18 aa, Feline leukemia provirus
LQNRRGLDILFLQEGGLC

SEQ ID NO: 135 - protein, 18 aa, Feline leukemia virus
LQNRRGLDILFLQEGGLC

SEQ ID NO: 136 - protein, 18 aa, Feline leukemia virus
LQNRRGLDILFLQEGGLC

SEQ ID NO: 137 - protein, 18 aa, Feline sarcoma virus
LQNRRGLDILFLQEGGLC

SEQ ID NO: 138 - protein, 18 aa, Feline sarcoma virus
LQNRRGLDILFLQGGGLC

SEQ ID NO: 139 - protein, 18 aa, Gibbon ape leukemia virus
LQNRRGLDLLFLKEGGLC

SEQ ID NO: 140 - protein, 18 aa, Human T-cell leukemia virus 1
AQNRRGLDLLFWEQGGLC

SEQ ID NO: 141 - protein, 18 aa, Human T-cell leukemia virus 1
AQNRRGLDLLFWEQGGLC

SEQ ID NO: 142 - protein, 18 aa, Human T-cell leukemia virus 1
AQNRRGLDLLFWEQGGLC

SEQ ID NO: 143 - protein, 18 aa, Human T-cell leukemia virus 1
AQNRRGLDLLFWEQGGLC

SEQ ID NO: 144 - protein, 18 aa, Human T-cell leukemia virus 1
AQNRRGLDLLFWEQGGLC

SEQ ID NO: 145 - protein, 17 aa, Human T-cell leukemia virus 2
QNRRGLDLLFWEQGGLC

SEQ ID NO: 146 - protein, 18 aa, Koala retrovirus
LQNRRGLDLLFLKEGGLC

SEQ ID NO: 147 - protein, 18 aa, Mink cell focus forming murine leukemia virus
LQNRRGLDLLFLKEGGLC

SEQ ID NO: 148 - protein, 18 aa, Mink cell focus-forming murine leukemia virus
LQNRRGLDLLFLKEGGLC

SEQ ID NO: 149 - protein, 18 aa, AKV murine leukemia virus
LQNRRGLDLLFLKEGGLC

SEQ ID NO: 150 - protein, 18 aa, CasBrE murine leukemia virus
LQNRRGLDLLFLKEGGLC

SEQ ID NO: 151 - protein, 18 aa, Friend murine leukemia virus
LQNRRGLDLLFLKEGGLC

SEQ ID NO: 152 - protein, 18 aa, Friend murine leukemia virus
LQNRRGLDLLFLKEGGLC

SEQ ID NO: 153 - protein, 18 aa, Friend murine leukemia virus
LQNRRGLDLLFLKEGGLC

SEQ ID NO: 154 - protein, 18 aa, Hortulanus murine leukemia virus
LQNRRGLDLLFLEKGGLC

SEQ ID NO: 155 - protein, 18 aa, Moloney murine leukemia virus
LQNRRGLDLLFLKEGGLC

SEQ ID NO: 156 - protein, 18 aa, Radiation murine leukemia virus
LQNRRGLDLLFLKEGGLC

SEQ ID NO: 157 - protein, 18 aa, Radiation murine leukemia virus
LQNRRGLDLLFLKEGGLC

SEQ ID NO: 158 - protein, 18 aa, Mason-Pfizer monkey virus
LQNRRGLDLLTAEQGGIC

SEQ ID NO: 159 - protein, 18 aa, Rauscher mink cell focus-inducing virus
LQNRRGLDLLFLKEGGLC

SEQ ID NO: 160 - protein, 18 aa, Rous sarcoma virus
LQNRAAIDFLLLAHGHGC

SEQ ID NO: 161 - protein, 18 aa, Squirrel monkey retrovirus
LQNRRGLDLLTAEQGGIC

SEQ ID NO: 162 - protein, 18 aa, Simian retrovirus SRV-1
LQNRRGLDLLTAEQGGIC

SEQ ID NO: 163 - protein, 18 aa, Simian retrovirus SRV-2
LQNRRGLDLLTAEQGGIC

SEQ ID NO: 164 - protein, 18 aa, Simian retrovirus SRV-2
LQNRRGLDLFTAEQGGIC

SEQ ID NO: 165 - protein, 18 aa, Mus musculus
LQNRRGLDLLFLKEGGLC

SEQ ID NO: 166 - protein, 18 aa, Homo sapiens
LQNRRALDLLTAERGGTC

SEQ ID NO: 167 - protein, 17 aa, Simian T-lymphotropic virus 2
QNRRGLDLLFWEQGGLC

SEQ ID NO: 168 - protein, 18 aa, Murine leukemia virus
LQNRRGLDLLFLKEGGLC

SEQ ID NO: 169 - protein, 18 aa, Human T-lymphotropic virus 1
AQNRRGLDLLFWEQGGLC

SEQ ID NO: 170 - protein, 18 aa, Lake Victoria marburgvirus
LINRHAIDFLLTRWGGTC

SEQ ID NO: 171 - protein, 18 aa, Lake Victoria marburgvirus
LINRHAIDFLLTRWGGTC

SEQ ID NO: 172 - protein, 18 aa, Friend murine leukemia virus
LQNRRGLDLLFLKEGGLC

SEQ ID NO: 173 - protein, 18 aa, Porcine endogenous retrovirus
LQNRRGLDLLFLKEGGLC

SEQ ID NO: 174 - protein, 18 aa, Porcine endogenous retrovirus
LQNRRGLDLLFLREGGLC

SEQ ID NO: 175 - protein, 18 aa, Rauscher murine leukemia virus
LQNRRGLDLLFLKEGGLC

SEQ ID NO: 176 - protein, 18 aa, Feline leukemia virus
LQNRRGLDILFLQEGGLC

SEQ ID NO: 177 - protein, 18 aa, Simian T-lymphotropic virus 1
AQNRRGLDLLFWEQGGLC

SEQ ID NO: 178 - protein, 18 aa, Sus scrofa
LQNRRGLDLLFLKEGGLC

SEQ ID NO: 179 - protein, 18 aa, Sus scrofa
LQNRRGLDLLFLKEGGLC

SEQ ID NO: 180 - protein, 17 aa, Simian T-lymphotropic virus 2
QNRRGLDLLFWEQGGLC

SEQ ID NO: 181 - protein, 18 aa, Gibbon ape leukemia virus
LQNRRGLDLLFLKEGGLC

SEQ ID NO: 182 - protein, 18 aa, Porcine endogenous retrovirus
LQNRRGLDLLFLREGGLC

SEQ ID NO: 183 - protein, 17 aa, Human T-cell lymphotropic virus type 2b
QNRRGLDLLFWEQGGLC

SEQ ID NO: 184 - protein, 18 aa, Porcine endogenous retrovirus
LQNRRGLDLLFLREGGLC

SEQ ID NO: 185 - protein, 18 aa, Feline leukemia virus
LQNRRGLDILFLQEGGLC

SEQ ID NO: 186 - protein, 18 aa, Mus dunni endogenous virus
LQNRRGLDLLFLKEGGLC

SEQ ID NO: 187 - protein, 18 aa, Friend spleen focus-forming virus
LQNRRGLDLLFLKEGGLC

SEQ ID NO: 188 - protein, 18 aa, Rous sarcoma virus
LQNRAAIDFLLLAHGHGC

SEQ ID NO: 189 - protein, 18 aa, Mus musculus
LQNRRGLDLLFLKEGGLC

SEQ ID NO: 190 - protein, 18 aa, Human T-lymphotropic virus 1
AQNRRGLDLLFWEQGGLC

SEQ ID NO: 191 - protein, 18 aa, Human T-lymphotropic virus 1
AQNRRGLDLLFWEQGGLC

SEQ ID NO: 192 - protein, 18 aa, Human T-lymphotropic virus 1
AQNRRGLDLLFWEQGGLC

SEQ ID NO: 193 - protein, 18 aa, Human T-lymphotropic virus 1
AQNRRGLDLLFWEQGGLC

SEQ ID NO: 194 - protein, 18 aa, Human T-lymphotropic virus 1
AQNRRGLDLLFWEQGGLC

SEQ ID NO: 195 - protein, 18 aa, Human T-lymphotropic virus 1
AQNRRGLDLLFWEQGGLC

SEQ ID NO: 196 - protein, 18 aa, Human T-lymphotropic virus 1
AQNRRGLDLLFWEQGGLC

SEQ ID NO: 197 - protein, 18 aa, Mus musculus
LRNQREQDFQSLQQDGLC

SEQ ID NO: 198 - protein, 18 aa, Retroviridae
LQNRRGLDLLFLKEGGLC

SEQ ID NO: 199 - protein, 18 aa, Retroviridae
LQNRRGLDLLFLKEGGLC

SEQ ID NO: 200 - protein, 18 aa, Avian leukosis virus RSA
LQNRAAIDFLLLAHGHGC

SEQ ID NO: 201 - protein, 18 aa, Human T-lymphotropic virus 1
AQNRRGLDLLFWEQGGLC

SEQ ID NO: 202 - protein, 18 aa, Rous sarcoma virus
LQNRAAIDFLLLAHGHGC

SEQ ID NO: 203 - protein, 18 aa, Simian T-lymphotropic virus 1
AQNRRGLDLLFWEQGGLC

SEQ ID NO: 204 - protein, 18 aa, Murine leukemia virus
LQNRRGLDLLFLKEGGLC

SEQ ID NO: 205 - protein, 18 aa, Mus musculus
LQNRRGLDLLFLKEGGLC

SEQ ID NO: 206 - protein, 18 aa, Rous sarcoma virus
LQNRAAIDFLLLAHGHGC

SEQ ID NO: 207 - protein, 17 aa, Human T-cell leukemia virus 2
QNRRGLDLLFWEQGGLC

SEQ ID NO: 208 - protein, 18 aa, Homo sapiens
YQNRLALDYLLAQEEGVC

SEQ ID NO: 209 - protein, 18 aa, Mus musculus
LQNRRGLDLLFLKEGGLC

SEQ ID NO: 210 - protein, 18 aa, Mus musculus
LQNRRGLDLLFLKEGGLC

SEQ ID NO: 211 - protein, 18 aa, Mus musculus
LQNRRGLDLLFLKEGGLC

SEQ ID NO: 212 - protein, 18 aa, Mus musculus
LQNRRGLDLLFLKEGGLC

SEQ ID NO: 213 - protein, 18 aa, Rous sarcoma virus
LQNRAAIDFLLLAHGHGC

SEQ ID NO: 214 - protein, 18 aa, Avian leukosis virus
LQNRAAIDFLLLAQGHGC

SEQ ID NO: 215 - protein, 18 aa, Baboon T-cell leukemia virus
AQNRRGLDLLFWEQGGLC

SEQ ID NO: 216 - protein, 18 aa, Zaire ebolavirus
ILNRKAIDFLLQRWGGTC

SEQ ID NO: 217 - protein, 18 aa, Feline leukemia virus
LQNRRGLDILFLQEGGLC

SEQ ID NO: 218 - protein, 18 aa, Friend murine leukemia virus
LQNRRGLDLLFLKEGGLC

SEQ ID NO: 219 - protein, 18 aa, Gibbon ape leukemia virus
LQNRRGLDLLFLKEGGLC

SEQ ID NO: 220 - protein, 18 aa, Avian leukosis virus
LQNRAAIDFLLLAQGHGC

SEQ ID NO: 221 - protein, 18 aa, Murine leukemia virus
LQNRRGLDLLFLKEGGLC

SEQ ID NO: 222 - protein, 18 aa, Human endogenous retrovirus
YQNRLALDYLLAAEGGVC

SEQ ID NO: 223 - protein, 18 aa, Amphotropic Murine leukemia virus
LQNRRGLDLLFLKEGGLC

SEQ ID NO: 224 - protein, 18 aa, Recombinant M-MuLV/RaLV retrovirus
LQNRRGLDMLFLREGGLC

SEQ ID NO: 225 - protein, 18 aa, Recombinant M-MuLV/RaLV retrovirus
LQNRRGLDMLFLREGGLC

SEQ ID NO: 226 - protein, 18 aa, Gallus gallus
LQNRAAIDFLLLAQGHGC

SEQ ID NO: 227 - protein, 18 aa, Human T-lymphotropic virus 1
AQNRRGLDLLFWEQGGLC

SEQ ID NO: 228 - protein, 18 aa, Human T-lymphotropic virus 1
AQNRRGLDLLFWEQGGLC

SEQ ID NO: 229 - protein, 18 aa, Human T-lymphotropic virus 1
AQNRRGLDLLFWEQGGLC

SEQ ID NO: 230 - protein, 18 aa, Human T-lymphotropic virus 1
AQNRRGLDLLFWEQGGLC

SEQ ID NO: 231 - protein, 17 aa, Human T-cell leukemia virus 2
QNRRGLDLLFWEQGGLC

SEQ ID NO: 232 - protein, 17 aa, Human T-cell leukemia virus 2
QNRRGLDLLFWEQGGLC

SEQ ID NO: 233 - protein, 18 aa, Avian myeloblastosis-associated virus
LQNRAAIDFLLLAHGHGC

SEQ ID NO: 234 - protein, 18 aa, Avian myeloblastosis-associated virus
LQNRAAIDFLLLAHGHGC

SEQ ID NO: 235 - protein, 18 aa, Avian myeloblastosis-associated virus type 2
LQNRAAIDFLLLAHGHGC

SEQ ID NO: 236 - protein, 18 aa, Murine leukemia virus
LQNRRGLDLLFLKEGGLC

SEQ ID NO: 237 - protein, 18 aa, Murine leukemia virus
LQNRRGLDLLFLKEGGLC

SEQ ID NO: 238 - protein, 18 aa, Murine leukemia virus
LQNRRGLDLLFLKEGGLC

SEQ ID NO: 239 - protein, 18 aa, Murine leukemia virus
LQNRRGLDLLFLKEGGLC

SEQ ID NO: 240 - protein, 18 aa, Rat leukemia virus
LQNRRGLDMLFLREGGLC

SEQ ID NO: 241 - protein, 18 aa, Murine leukemia virus
LQNRRGLDLLFLKEGGLC

SEQ ID NO: 242 - protein, 18 aa, Murine leukemia virus
LQNRRGLDLLFLKEGGLC

SEQ ID NO: 243 - protein, 17 aa, Simian T-lymphotropic virus 3
QNRRGLDLLFWEQGGLC

SEQ ID NO: 244 - protein, 18 aa, Murine leukemia virus
LQNRRGLDLLFLKEGGLC

SEQ ID NO: 245 - protein, 18 aa, Xenotropic murine leukemia virus
LQNRRGLDLLFLKEGGLC

SEQ ID NO: 246 - protein, 18 aa, Feline leukemia virus
LQNRRGLDILFLQEGGLC

SEQ ID NO: 247 - protein, 18 aa, Feline leukemia virus
LQNRRGLDILFLQEGGLC

SEQ ID NO: 248 - protein, 18 aa, Feline leukemia virus
LQNRRGLDILFLQEGGLC

SEQ ID NO: 249 - protein, 18 aa, Friend mink cell focus-inducing virus
LQNRRGLDLLFLKEGGLC

SEQ ID NO: 250 - protein, 18 aa, Murine type C retrovirus
LQNRRGLDLLFLKEGGLC

SEQ ID NO: 251 - protein, 17 aa, Human T-cell leukemia virus 2
QNRRGLDLLFWEQGGLC

SEQ ID NO: 252 - protein, 18 aa, Homo sapiens
YQNRLALDYLLAAEGGVC

SEQ ID NO: 253 - protein, 18 aa, Murine leukemia virus
LQNRRGLDLLFLKEGGLC

SEQ ID NO: 254 - protein, 17 aa, Simian T-lymphotropic virus 3
QNRRGLDLLFWEQGGLC

SEQ ID NO: 255 - protein, 18 aa, Python molurus endogenous retrovirus
LQNRRGLDLLFLKEGGLC

SEQ ID NO: 256 - protein, 18 aa, Python molurus endogenous retrovirus
LQNRRGLDLLFLKEGGLC

SEQ ID NO: 257 - protein, 18 aa, Zaire ebolavirus
ILNRKAIDFLLQRWGGTC

SEQ ID NO: 258 - protein, 18 aa, Mus musculus
LQNRRALDLITAEKGGTC

SEQ ID NO: 259 - protein, 18 aa, Mus musculus
LRNQREQDFQSLQQDGLC

SEQ ID NO: 260 - protein, 18 aa, Porcine endogenous retrovirus B
LQNRRGLDLLFLREGGLC

SEQ ID NO: 261 - protein, 18 aa, Porcine endogenous retrovirus A
LQNRRGLDLLFLKEGGLC

SEQ ID NO: 262 - protein, 18 aa, Porcine endogenous retrovirus
LQNRRGLDLLFLKEGGLC

SEQ ID NO: 263 - protein, 18 aa, Gallus gallus
LQNRMALDLLTAKEGGVC

SEQ ID NO: 264 - protein, 18 aa, Brachydanio rerio
IQNRLALDMLLSERGGVC

SEQ ID NO: 265 - protein, 18 aa, Reston ebolavirus
LLNRKAIDFLLQRWGGTC

SEQ ID NO: 266 - protein, 18 aa, Zaire ebolavirus
ILNRKAIDFLLQRWGGTC

SEQ ID NO: 267 - protein, 18 aa, Mus musculus
LQNRRGLDLLFLKEGGLC

SEQ ID NO: 268 - protein, 18 aa, Pan troglodytes
MQNRRALDLLTADKGGTC

SEQ ID NO: 269 - protein, 18 aa, Homo sapiens
LQNRRGLDMLTAAQGGIC

SEQ ID NO: 270 - protein, 18 aa, Porcine endogenous retrovirus
LQNRRGLDLLFLKEGGLC

SEQ ID NO: 271 - protein, 18 aa, Porcine endogenous retrovirus
LQNRRGLDLLFLKEGGLC

SEQ ID NO: 272 - protein, 18 aa, Porcine endogenous retrovirus
LQNRRGLDLLFLKEGGLC

SEQ ID NO: 273 - protein, 18 aa, Porcine endogenous retrovirus
LQNRRGLDLLFLKEGGLC

SEQ ID NO: 274 - protein, 18 aa, Porcine endogenous retrovirus
LQNRRGLDLLFLKEGGLC

SEQ ID NO: 275 - protein, 18 aa, Porcine endogenous retrovirus
LQNRRGLDLLFLKEGGLC

SEQ ID NO: 276 - protein, 18 aa, Porcine endogenous retrovirus
LQNRRGLDLLFLKEGGLC

SEQ ID NO: 277 - protein, 18 aa, Brachydanio rerio
IQNRLALDMLLSERGGVC

SEQ ID NO: 278 - protein, 18 aa, Mus musculus
LRNQREQDFQSLQQDGLC

SEQ ID NO: 279 - protein, 18 aa, Porcine endogenous retrovirus
LQNRRGLDLLFLREGGLC

SEQ ID NO: 280 - protein, 18 aa, Porcine endogenous retrovirus
LQNRRGLDLLFLKEGGLC

SEQ ID NO: 281 - protein, 18 aa, Porcine endogenous retrovirus
LQNRRGLDLLFLREGGLC

SEQ ID NO: 282 - protein, 18 aa, Moloney murine leukemia virus
LQNRRGLDLLFLKEGGLC

SEQ ID NO: 283 - protein, 18 aa, Human T-lymphotropic virus
  MOD_RES (10)..(10): Variable amino acid
AQNRRGLDLXFWEQGGLC

SEQ ID NO: 284 - protein, 17 aa, Human T-lymphotropic virus
QNRRGLDLLFWEQGGLC

SEQ ID NO: 285 - protein, 18 aa, Human T-lymphotropic virus
  MOD_RES (10)..(10): Variable amino acid
AQNRRGLDLXFWEQGGLC

SEQ ID NO: 286 - protein, 18 aa, Human T-lymphotropic virus
  MOD_RES (10)..(10): Variable amino acid
AQNRRGLDLXFWEQGGLC

SEQ ID NO: 287 - protein, 18 aa, Human T-lymphotropic virus
  MOD_RES (10)..(10): Variable amino acid
AQNRRGLDLXFWEQGGLC

SEQ ID NO: 288 - protein, 18 aa, Human T-lymphotropic virus
AQNRRGLDLLFWEQGGLC

SEQ ID NO: 289 - protein, 18 aa, Human T-lymphotropic virus
  MOD_RES (10)..(10): Variable amino acid
AQNRRGLDLXFWEQGGLC

SEQ ID NO: 290 - protein, 18 aa, Human T-lymphotropic virus
AQNRRGLDLLFWEQGGLC

SEQ ID NO: 291 - protein, 18 aa, Human T-lymphotropic virus
AQNRRGLDLLFWEQGGLC

SEQ ID NO: 292 - protein, 17 aa, Human T-lymphotropic virus
QNRRGLDLLFWEQGGLC

SEQ ID NO: 293 - protein, 18 aa, Human T-lymphotropic virus
  MOD_RES (10)..(10): Variable amino acid
AQNRRGLDLXFWEQGGLC

SEQ ID NO: 294 - protein, 18 aa, Feline leukemia virus
LQNRRGLDILFLQEGGLC

SEQ ID NO: 295 - protein, 18 aa, Avian leukosis virus
LQNRAAIDFLLLAQGHGC

SEQ ID NO: 296 - protein, 18 aa, Porcine endogenous type C retrovirus
LQNKKGLDLLFLKKRRLC

SEQ ID NO: 297 - protein, 18 aa, Porcine endogenous type C retrovirus
LQNRRGLDLLFLKEGGLC

SEQ ID NO: 298 - protein, 18 aa, Porcine endogenous type C retrovirus
LQNRRGLDLLFLKEGGLC

SEQ ID NO: 299 - protein, 18 aa, Porcine endogenous type C retrovirus
LQNRRGLDLLFLKEGGLC

SEQ ID NO: 300 - protein, 18 aa, Feline leukemia virus
LQNRRGLDILFLQGGGLC

SEQ ID NO: 301 - protein, 18 aa, Murine leukemia virus
LQNRRGLDLLFLKEGGLC

SEQ ID NO: 302 - protein, 18 aa, Porcine endogenous type C retrovirus
LQNRRGLDLLFLREGGLC

SEQ ID NO: 303 - protein, 18 aa, Porcine endogenous type C retrovirus
LQNRRGLDLLFLKEGGLC

SEQ ID NO: 304 - protein, 18 aa, Zaire ebolavirus
ILNRKAIDFLLQRWGGTC

SEQ ID NO: 305 - protein, 18 aa, Reston ebolavirus
LLNRKAIDFLLQRWGGTC

SEQ ID NO: 306 - protein, 18 aa, Mus musculus
LRNQREQDFQSLQQDGLC

SEQ ID NO: 307 - protein, 18 aa, Mus musculus castaneus
LQNRRGLDLLFLKEGGLC

SEQ ID NO: 308 - protein, 18 aa, Homo sapiens
MNNRLALDYLLAEQGGVC

SEQ ID NO: 309 - protein, 18 aa, RD114 retrovirus
LQNRRGLDLLTAEQGGIC

SEQ ID NO: 310 - protein, 18 aa, Avian leukosis virus
LQNRAAIDFLLLAHGHGC

SEQ ID NO: 311 - protein, 18 aa, Avian leukosis virus
LQNRAAIDFLLLAHGHGC

SEQ ID NO: 312 - protein, 18 aa, Xenotropic murine leukemia virus
LQNRRGLDLLFLKEGGLC

SEQ ID NO: 313 - protein, 18 aa, Multiple sclerosis associated retrovirus element
LQNRRALDLLTAKRGGTC

SEQ ID NO: 314 - protein, 18 aa, Mus cervicolor popaeus endogenous virus
LQNRRGLDLLFLKEGGLC

SEQ ID NO: 315 - protein, 18 aa, Mus musculus
LQNRQGLDVLSAKEGGLC

SEQ ID NO: 316 - protein, 17 aa, Human T-cell leukemia virus 2
QNRRGLDLLFWEQGGLC

SEQ ID NO: 317 - protein, 17 aa, Human T-cell leukemia virus 2
QNRRGLDLLFWEQGGLC

SEQ ID NO: 318 - protein, 18 aa, Avian leukosis virus
LQNRAAIDFLLLAQGHGC

SEQ ID NO: 319 - protein, 18 aa, Avian leukosis virus
LQNRAAIDFLLLAQGHGC

SEQ ID NO: 320 - protein, 18 aa, Avian leukosis virus
LQNRAAIDFLLLAQGHGC

SEQ ID NO: 321 - protein, 18 aa, Avian leukosis virus
LQNRAAIDFLLLAQGHGC

SEQ ID NO: 322 - protein, 18 aa, Murine leukemia virus
LQNRRGLDLLFLKEGGLC

SEQ ID NO: 323 - protein, 18 aa, Murine leukemia virus
LQNRRGLDLLFLKEGGLC

SEQ ID NO: 324 - protein, 18 aa, Murine leukemia virus
LQNRRGLDLLFLKEGGLC

SEQ ID NO: 325 - protein, 18 aa, Murine leukemia virus
LQNRRGLDLLFLKEGGLC

SEQ ID NO: 326 - protein, 18 aa, DG-75 Murine leukemia virus
LQNRRGLDLLFLKEGGLC

SEQ ID NO: 327 - protein, 18 aa, Trichosurus vulpecula
LQNRRGLDLLTAEQGGIC

SEQ ID NO: 328 - protein, 18 aa, Fowlpox virus
LQNRRGLDLLTAEQGGIC

SEQ ID NO: 329 - protein, 18 aa, Porcine endogenous retrovirus
LQNRRGLDLLFLREGGLC

SEQ ID NO: 330 - protein, 18 aa, Porcine endogenous retrovirus
LQNRRGLDLLFLREGGLC

SEQ ID NO: 331 - protein, 18 aa, Porcine endogenous retrovirus
LQNRRGLDLLFLKEGGLC

SEQ ID NO: 332 - protein, 18 aa, Porcine endogenous retrovirus
LQNRRGLDLLFLKEGGLC

SEQ ID NO: 333 - protein, 18 aa, Human T-lymphotropic virus 1
AQNRRGLDLLFWEQGGLC

SEQ ID NO: 334 - protein, 18 aa, Human T-lymphotropic virus 1
AQNRRGLDLLFWEQGGLC

SEQ ID NO: 335 - protein, 18 aa, Homo sapiens
LQNRQGLDLLTAEKGGLC

SEQ ID NO: 336 - protein, 18 aa, Homo sapiens
LQNRRGLDLLTAEKGGLC

SEQ ID NO: 337 - protein, 18 aa, Homo sapiens
LRNRRALDLLTAERGGTC

SEQ ID NO: 338 - protein, 18 aa, Homo sapiens
LQNRRALDLLTAERGGTC

SEQ ID NO: 339 - protein, 18 aa, Gallus gallus
LQNRAVIDFLLLAQGHGC

SEQ ID NO: 340 - protein, 18 aa, Human T-lymphotropic virus 1
AQNRRRLDLLFWEQGGLC

SEQ ID NO: 341 - protein, 18 aa, Porcine endogenous retrovirus
LQNRRGLDLLFLREGGLC

SEQ ID NO: 342 - protein, 18 aa, Porcine endogenous retrovirus
LQNRRGLDLLFLKEGGLC

SEQ ID NO: 343 - protein, 18 aa, Porcine endogenous retrovirus
LQNRRGLDLLFLREGGLC

SEQ ID NO: 344 - protein, 18 aa, Murine leukemia virus
LQNRRGLDLLFLKEGGLC

SEQ ID NO: 345 - protein, 18 aa, Porcine endogenous type C retrovirus
LQNRRGLDLLFLKEGGLC

SEQ ID NO: 346 - protein, 18 aa, Avian endogenous retrovirus
LQNRAAIDFLLLAQGHGC

SEQ ID NO: 347 - protein, 18 aa, Koala retrovirus
LQNRRGLDLLFLKEGGLC

SEQ ID NO: 348 - protein, 18 aa, Homo sapiens
LQNRRGLDLLTAEKGGLC

SEQ ID NO: 349 - protein, 18 aa, Homo sapiens
LQNRRALDLLTAERGGTC

SEQ ID NO: 350 - protein, 18 aa, Friend mink cell focus-inducing virus
LQNRRGLDLLFLKEGGLC

SEQ ID NO: 351 - protein, 18 aa, Murine leukemia virus
LQNRRGLDLLFLKEGGLC

SEQ ID NO: 352 - protein, 17 aa, Human T-cell leukemia virus 2
QNRRGLDLLFWEQGGLC

SEQ ID NO: 353 - protein, 18 aa, Murine leukemia virus
LQNRRGLDLLFLKEGGLC

SEQ ID NO: 354 - protein, 18 aa, Simian T-lymphotropic virus 1
AQNRRGLDLLFWEQGGLC

SEQ ID NO: 355 - protein, 17 aa, Human T-cell leukemia virus 2
QNRRGLDLLFWEQGGLC

SEQ ID NO: 356 - protein, 18 aa, Felis silvestris catus
LQNRRGLDLLFLQEGGLC

SEQ ID NO: 357 - protein, 18 aa, Simian sarcoma-associated virus
LQNRRGLDLLFLKEGGLC

SEQ ID NO: 358 - protein, 18 aa, Gibbon ape leukemia virus
LQNRRGLDLLFLKEGGLC

SEQ ID NO: 359 - protein, 18 aa, Gibbon ape leukemia virus
LQNRRGLDLLFLKEGGLC

SEQ ID NO: 360 - protein, 18 aa, Gibbon ape leukemia virus
LQNRRGLDLLFLKEGGLC

SEQ ID NO: 361 - protein, 18 aa, Gibbon ape leukemia virus
LQNRRGLDLLFLKEGGLC

SEQ ID NO: 362 - protein, 18 aa, Murine leukemia virus
LQNRRGLDLLFLKEGGLC

SEQ ID NO: 363 - protein, 18 aa, Zaire ebolavirus
ILNRKAIDFLLQRWGGTC

SEQ ID NO: 364 - protein, 18 aa, Zaire ebolavirus
ILNRKAIDFLLQRWGGTC

SEQ ID NO: 365 - protein, 18 aa, Ivory Coast ebolavirus
ILNRKAIDFLLQRWGGTC

SEQ ID NO: 366 - protein, 18 aa, Reston ebolavirus
LLNRKAIDFLLQRWGGTC

SEQ ID NO: 367 - protein, 18 aa, Reston ebolavirus
LLNRKAIDFLLQRWGGTC

SEQ ID NO: 368 - protein, 18 aa, Sudan ebolavirus
LLNRKAIDFLLQRWGGTC

SEQ ID NO: 369 - protein, 18 aa, Sudan ebolavirus
ILNRKAIDFLLRRWGGTC

SEQ ID NO: 370 - protein, 18 aa, Sudan ebolavirus
ILNRKAIDFLLRRWGGTC

SEQ ID NO: 371 - protein, 18 aa, Zaire ebolavirus
ILNRKAIDFLLQRWGGTC

SEQ ID NO: 372 - protein, 18 aa, Zaire ebolavirus
ILNRKAIDFLLQRWGGTC

SEQ ID NO: 373 - protein, 18 aa, Zaire ebolavirus
LINRHAIDFLLTRWGGTC

SEQ ID NO: 374 - protein, 18 aa, Zaire ebolavirus
LINRHAIDFLLTRWGGTC

SEQ ID NO: 375 - protein, 18 aa, Lake Victoria marburgvirus
LINRHAIDFLLTRWGGTC

SEQ ID NO: 376 - protein, 18 aa, Lake Victoria marburgvirus
LINRHAIDFLLTRWGGTC

SEQ ID NO: 377 - protein, 16 aa, Artificial Sequence
  Description of Artificial Sequence: Synthetic peptide
  MOD_RES (2)..(2): Arg or Lys
  MOD_RES (3)..(4): Variable amino acid
  MOD_RES (5)..(5): Leu or Ile
  MOD_RES (7)..(7): Variable amino acid
  MOD_RES (9)..(9): Leu, Ile or Phe
  MOD_RES (10)..(10): Variable amino acid
  MOD_RES (11)..(11): Arg or Lys
  MOD_RES (12)..(15): Variable amino acid
NXXXXDXLXXXXXXXC

SEQ ID NO: 378 - protein, 16 aa, Artificial Sequence
  Description of Artificial Sequence: Synthetic peptide
  MOD_RES (2)..(2): Arg or Lys
  MOD_RES (3)..(4): Variable amino acid
  MOD_RES (5)..(5): Leu or Ile
  MOD_RES (7)..(7): Variable amino acid
  MOD_RES (9)..(9): Leu, Ile or Phe
  MOD_RES (10)..(10): Variable amino acid
  MOD_RES (11)..(11): Arg or Lys
NXXXXDXLXXXWGGTC

SEQ ID NO: 379 - protein, 18 aa, Artificial Sequence
  Description of Artificial Sequence: Synthetic peptide
  MOD_RES (1)..(1): Ile or Leu
  MOD_RES (2)..(2): Leu or Ile
  MOD_RES (4)..(4): Arg or Lys
  MOD_RES (5)..(6): Variable amino acid
  MOD_RES (7)..(7): Leu or Ile
  MOD_RES (9)..(9): Variable amino acid
  MOD_RES (11)..(11): Leu, Ile or Phe
  MOD_RES (12)..(12): Variable amino acid
  MOD_RES (13)..(13): Arg or Lys
XXNXXXXDXLXXXWGGTC

SEQ ID NO: 380 - protein, 85 aa, Mus musculus
QLSKVLSETLEEIAASITTLQNQIDSLAGVVLQNRRALDLITAEKGGTCLFLQEECCFYVNQSGIVRDAARKLQERASELGQHSD

SEQ ID NO: 381 - protein, 95 aa, Moloney-Murine Leukemia Virus
NEIARIKKLIGEADGLIEGLRQLANETTQALQLFLRATTELRTFSILNRKAIDFLLQRWGGTCHILGPDCRIEPHDWTKNITDKIDQIIHDFVDK